SEQUENCE LISTING (1 sequence)

SEQ ID NO: 1 - protein, 1114 aa, Homo sapiens
MAKATSGAAGLRLLLLLLLPLLGKVALGLYFSRDAYWEKLYVDQAAGTPLLYVHALRDAPEEVPSFRLGQHLYGTYRTRLHENNWICIQEDTGLLYLNRSLDHSSWEKLSVRNRGFPLLTVYLKVFLSPTSLREGECQWPGCARVYFSFFNTSFPACSSLKPRELCFPETRPSFRIRENRPPGTFHQFRLLPVQFLCPNISVAYRLLEGEGLPFRCAPDSLEVSTRWALDREQREKYELVAVCTVHAGAREEVVMVPFPVTVYDEDDSAPTFPAGVDTASAVVEFKRKEDTVVATLRVFDADVVPASGELVRRYTSTLLPGDTWAQQTFRVEHWPNETSVQANGSFVRATVHDYRLVLNRNLSISENRTMQLAVLVNDSDFQGPGAGVLLLHFNVSVLPVSLHLPSTYSLSVSRRARRFAQIGKVCVENCQAFSGINVQYKLHSSGANCSTLGVVTSAEDTSGILFVNDTKALRRPKCAELHYMVVATDQQTSRQAQAQLLVTVEGSYVAEEAGCPLSCAVSKRRLECEECGGLGSPTGRCEWRQGDGKGITRNFSTCSPSTKTCPDGHCDVVETQDINICPQDCLRGSIVGGHEPGEPRGIKAGYGTCNCFPEEEKCFCEPEDIQDPLCDELCRTVIAAAVLFSFIVSVLLSAFCIHCYHKFAHKPPISSAEMTFRRPAQAFPVSYSSSGARRPSLDSMENQVSVDAFKILEDPKWEFPRKNLVLGKTLGEGEFGKVVKATAFHLKGRAGYTTVAVKMLKENASPSELRDLLSEFNVLKQVNHPHVIKLYGACSQDGPLLLIVEYAKYGSLRGFLRESRKVGPGYLGSGGSRNSSSLDHPDERALTMGDLISFAWQISQGMQYLAEMKLVHRDLAARNILVAEGRKMKISDFGLSRDVYEEDSYVKRSQGRIPVKWMAIESLFDHIYTTQSDVWSFGVLLWEIVTLGGNPYPGIPPERLFNLLKTGHRMERPDNCSEEMYRLMLQCWKQEPDKRPVFADISKDLEKMMVKRRDYLDLAASTPSDSLIYDDGLSEEETPLVDCNNAPLPRALPSTWIENKLYGMSDPNWPGESPVPLTRADGTNTGFPRYPNDSVYANWMLSPSAAKLMDTFDS

(19) United States
(12) Patent Application Publication
(10) Pub. No.: US 2026/0226039 A1
(43) Pub. Date: Aug. 6, 2026

(54) INHIBITORS OF SOLVENT FRONT MUTATED RET KINASE

(71) Applicants: Purdue Research Foundation, West Lafayette, IN (US); The Board of Regents of the University of Oklahoma, Norman, OK (US)

(72) Inventors: Herman O. Sintim, West Lafayette, IN (US); Neetu Dayal, Agra, Uttar Pradesh (IN); Elizabeth Anne Larocque, Lowell, MA (US); Jie Wu, Edmond, OK (US)

(21) Appl. No.: 19/150,513

(22) PCT Filed: Jan. 23, 2024
(86) PCT No.: PCT/US2024/012534

§ 371 Date: Jul. 23, 2025

Related U.S. Application Data
(60) Provisional application No. 63/440,542, filed on Jan. 23, 2023.

(51) Int. Cl. C07D 413/14; A61K 31/496; A61K 31/497; A61K 31/501; A61K 31/506; A61K 31/5377; A61K 31/541; A61P 35/00; C07D 401/06; C07D 401/14; C07D 471/04; C07D 487/10; C07D 491/107

(52) U.S. Cl. CPC C07D 413/14; A61K 31/496; A61K 31/497; A61K 31/501; A61K 31/506; A61K 31/5377; A61K 31/541; A61P 35/00; C07D 401/06; C07D 401/14; C07D 471/04; C07D 487/10; C07D 491/107

(57) ABSTRACT

Alkynyl nicotinamide compounds that inhibit Rearranged During Transfection (RET) kinase mutants resistant to RET protein tyrosine kinase inhibitors (RET-TKIs); compositions comprising same; and their use for treating RET kinase-altered cancers.

### CROSS-REFERENCE TO RELATED APPLICATION

This application claims priority to U.S. provisional patent application No. 63/440,542, which was filed Jan. 23, 2023, and which is hereby incorporated by reference in its entirety.

### GOVERNMENT RIGHTS

This invention was made with government support under CA273168 awarded by the National Institutes of Health. The government has certain rights in the invention.

### TECHNICAL FIELD

The present disclosure relates to alkynyl nicotinamide compounds that are inhibitors of Rearranged During Transfection (RET) kinase and their use in treating RET kinase-driven cancers. Particularly, the present disclosure relates to alkynyl nicotinamide compounds that are inhibitors of RET kinase solvent front mutations.

### STATEMENT OF SEQUENCE LISTING

A computer-readable form (CRF) of the Sequence Listing is submitted concurrently with this application, which is herein incorporated by reference in its entirety. The content of the computer-readable form is identical to the written sequence listing set forth herein.

### BACKGROUND

This section introduces aspects that may help facilitate a better understanding of the disclosure. Accordingly, these statements are to be read in this light and are not to be construed as admissions about what is or is not prior art.

Inhibitors of protein kinases can potentially treat many diseases controlled by the disregulation of protein kinases. Many alkynyl-containing compounds that inhibit kinases, such as olverembatinin and ponatinib, have been disclosed in the prior art (e.g., J. Med. Chem. 2017, 60, 8801-8815; Future Med Chem. 2017, 9(11) 1213-1225; and ChemMedChem 2018, 13, 1172-1180). The alkynyl benzamide-containing compounds are highly protein bound and have poor aqueous solubility. For example, ponatinib strongly inhibits human Ether-h-go-go-Related Gene (hERG) with an IC₅₀~1 μM and greater than 99% protein bound.

It has been disclosed that alkynyl benzamide-containing kinase inhibitors increase aqueous solubility when the benzamide group is converted into a nicotinamide group (e.g., ACS Omega 2020, 5, 2690-2698, and Int'l Pat. App. Pub. No. WO 2013/170770). The nature of the hinge-binding moiety in alkynyl nicotinamides is critical for kinase binding and drug properties such as solubility, hERG binding, and pharmacokinetics. Considering that there are multitudes of heteroaromatics, including type and substitution patterns that could be combined with the nicotinamide moiety, it is non-trivial to predict a priori which combinations of nicotinamide and heteroaromatic moiety lead to compounds that potently inhibit a specific kinase (over 500 possibilities) and mutants that arise in the clinic. Thus, prior disclosures do not necessarily facilitate the identification of a compound that would work in vivo with desired physicochemical properties.

Selpercatinib (LOXO292) and pralsetinib (BLU667) are protein tyrosine kinase inhibitors (TKIs), which exhibit specificity for Rearranged During Transfection (RET) kinase and have been recently approved for treating RET kinase-altered cancers. RET kinase is a transmembrane receptor protein-tyrosine kinase that activates multiple downstream pathways involved in cell proliferation and survival. However, RET kinase mutations that confer selpercatinib and pralsetinib resistance have been identified preclinically and in cancer patients. RET kinase G810 mutants are resistant to selpercatinib and pralsetinib. RET kinase G810 solvent front mutations represent the first described recurrent mechanism of resistance to selective RET kinase inhibition with selpercatinib. The development of a potent inhibitor of these mutations and the maintenance of activity against RET kinase gatekeeper mutations could be an effective strategy to target resistance to selective RET kinase inhibitors.

Therefore, there is an unmet need to develop a compound that potently inhibits RET kinase mutants such as G810. It is an object of the present disclosure to provide such a compound. This and other objects and advantages, as well as inventive features, will be apparent from the detailed description.

### SUMMARY

Provided is a compound of formula (I), or a pharmaceutically acceptable salt, hydrate, tautomer, or optical isomer thereof:

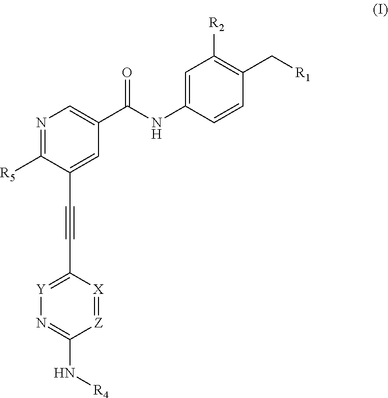

wherein
R₁ is NRₐR_{b}, wherein Rₐ and R_{b} each independently represent a group selected from H, alkyl, cycloalkyl, heterocycloalkyl, aryl, heteroaryl, —(CH₂)ₚ-alkyl, —(CH₂)ₚ-cycloalkyl. —(CH₂)ₚ-heterocycloalkyl, —(CH₂)ₚ—SO₂R_{c}, —(CH₂)ₚ—OR_{c}, and —(CH₂)ₚ—NRₐR_{b}, wherein Rₐ and R_{b} each independently is optionally substituted with at least one of alkyl, halo, OR_{c}, and NH₂; or
Rₐ and R_{b}, together with the nitrogen atom to which they are attached, form a 4- to 8-membered heterocycloalkyl group, which is a mono or a bicyclic ring, either of which is optionally substituted with a group selected from alkyl, cycloalkyl, halo, nitro, CN, SO₂R_{c}, OR_{d}, —(CH₂)ₚ—OR_{c} and NR_{c}R_{c};
R_{c} is H or alkyl;
R_{d} is H, halo, OH, CN, alkyl, —(CH₂)ₚ-alkyl, —(CH₂)ₚ-cycloalkyl, —(CH₂)ₚ-heterocycloalkyl, —(CH₂)ₚ—SO₂R_{d}, —(CH₂)ₚ—OR_{d}, —(CH₂)ₚ—(OR_{d})₂, or —(CH₂)ₚ—NRₐR_{b};
p is 0-5;
R₂ represents a group independently selected from alkyl, CF₃, OCF₃, and halo;
X, Y, and Z each independently represent a group selected from CR₃ and N with the proviso that the ring of which X, Y, and Z is comprised contains at least two N atoms, wherein R₃ is H, alkyl, ORₐ, or NRₐR_{b}, wherein Rₐ and R_{b} are as defined above;
R₄ represents a group selected from H, halo, OH, CN, alkyl, —(CH₂)ₚ-alkyl, —(CH₂)ₚ-cycloalkyl, —(CH₂)ₚ-heterocycloalkyl, —(CH₂)ₚ—SO₂Rₐ, —(CH₂)ₚ—OR_{d}, —(CH₂)ₚ—(ORₐ)₂, and —(CH₂)ₚ—NRₐR_{b}, wherein Rₐ, R_{b}, R_{d}, and p are as defined above; and
R₅ represents a group independently selected from H, alkyl, and halo.

In some embodiments, each X, Y, and Z is independently a group selected from CR₃ and N with the proviso that the ring of which X, Y, and Z is comprised contains at least two N atoms, wherein R₃ represents a group selected from:

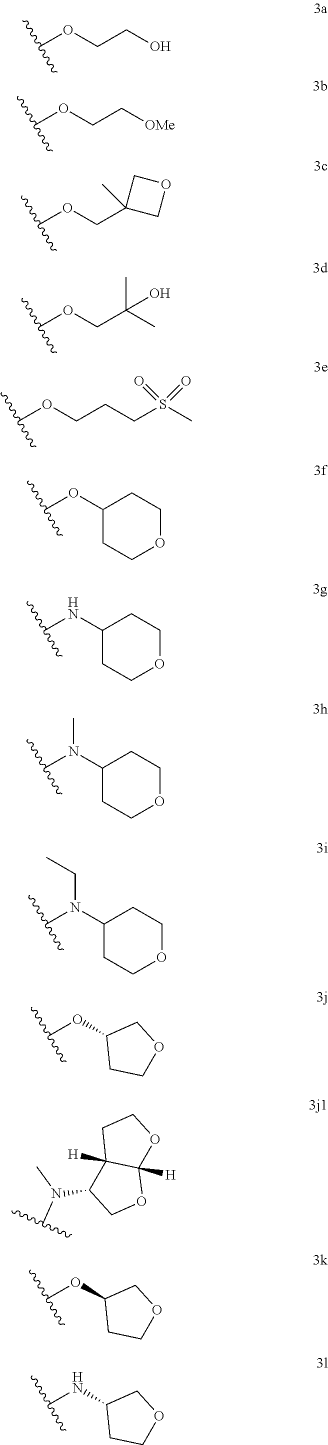

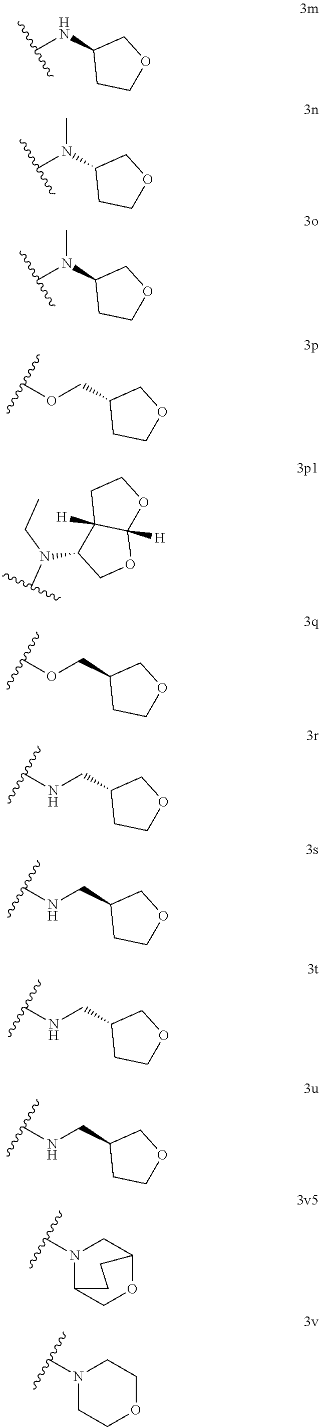

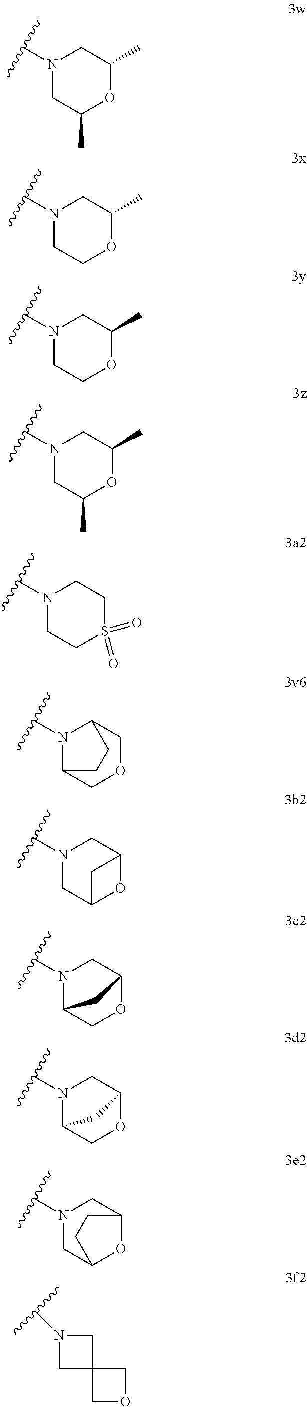

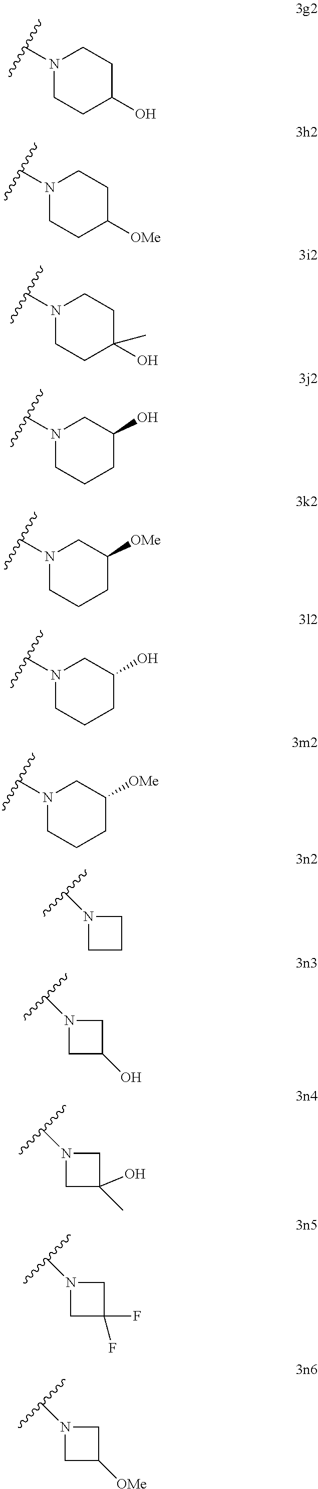

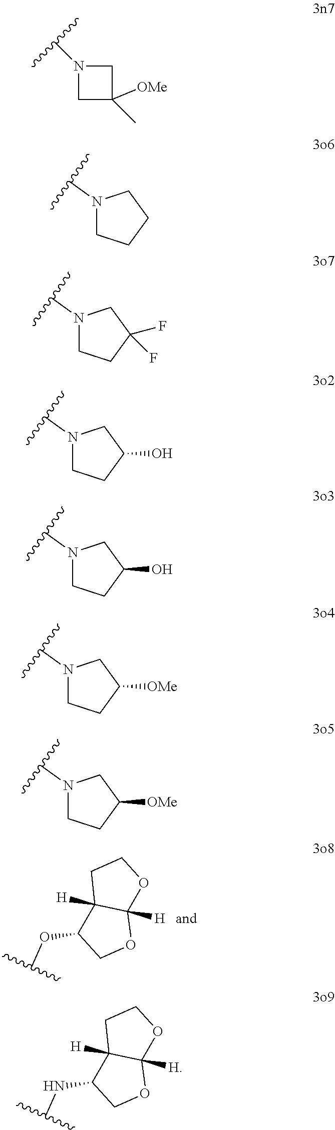

In some embodiments, R₄ is alkyl or a group selected from:

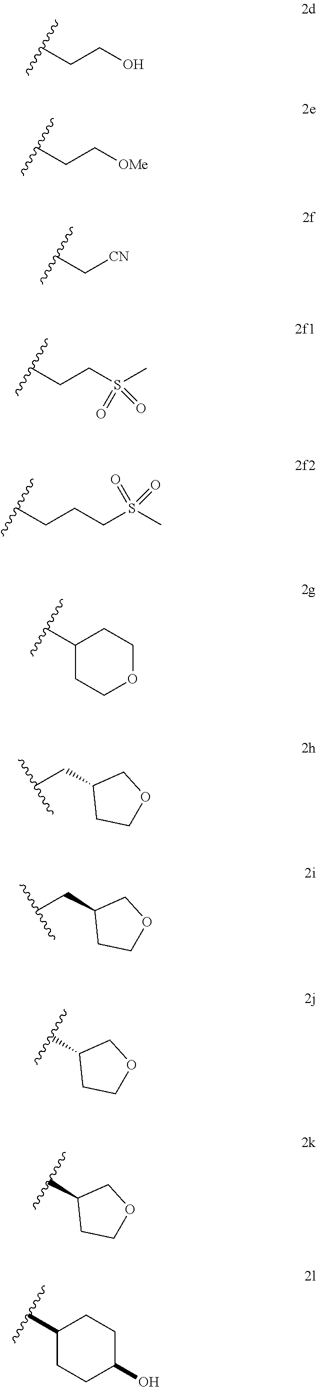

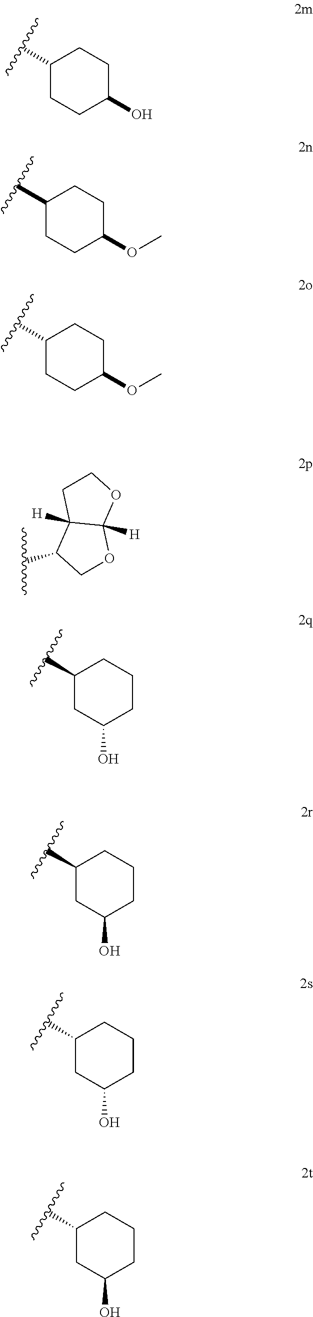

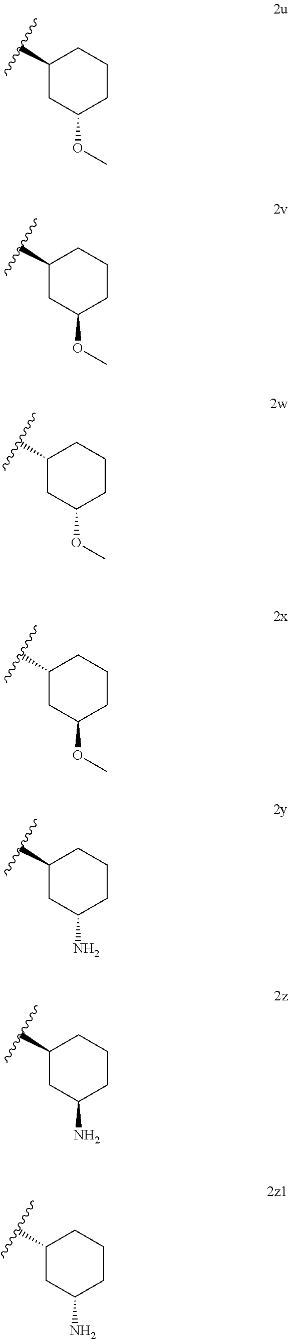

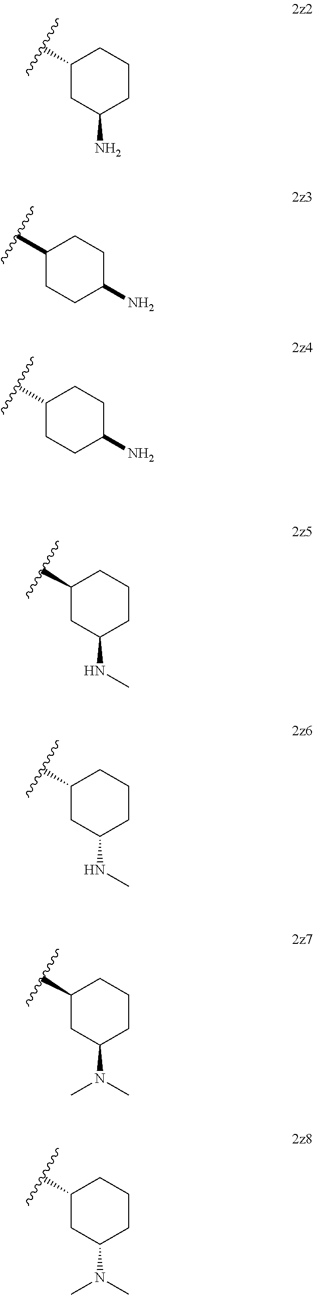

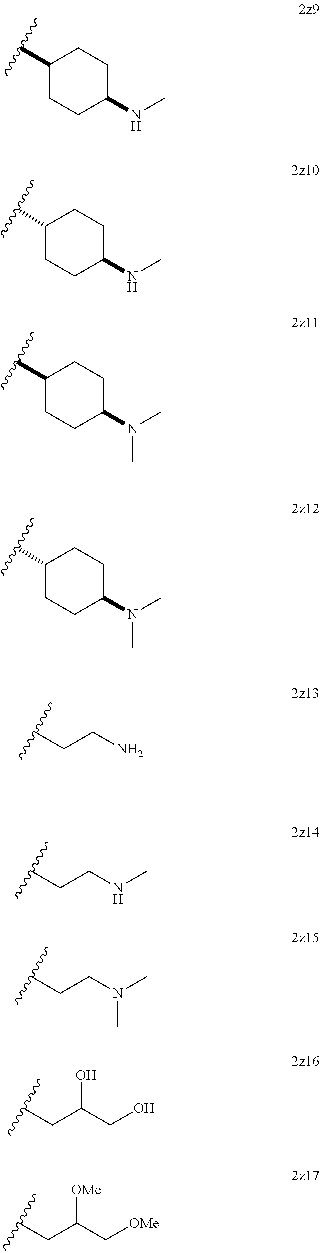

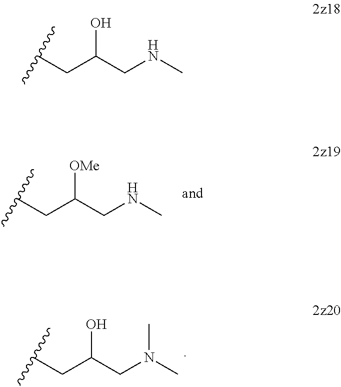

In some embodiments, the compound of formula (I) is 5-((2-aminopyrimidin-5-yl)ethynyl)-N-(4-((4-methylpiperazin-1-yl)methyl)-3-(trifluoromethyl)phenyl)nicotinamide,

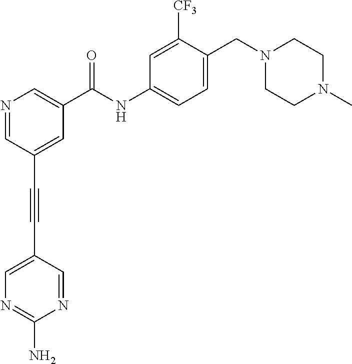 [SMILES: CN1CCN(Cc2ccc(NC(=O)c3cncc(C#Cc4cnc(N)nc4)c3)cc2C(F)(F)F)CC1]

or a pharmaceutically acceptable salt, hydrate, tautomer, or optical isomer thereof.

In some embodiments, the compound of formula (I) is 5-((3-amino-1,2,4-triazin-5-yl)ethynyl)-N-(4-((4-methylpiperazin-1-yl)methyl)-3-(trifluoromethyl)phenyl)nicotinamide,

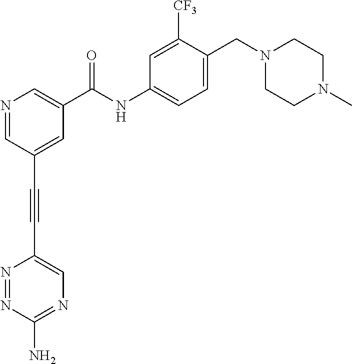 [SMILES: CN1CCN(Cc2ccc(NC(=O)c3cncc(C#Cc4cnc(N)nn4)c3)cc2C(F)(F)F)CC1]

or a pharmaceutically acceptable salt, hydrate, tautomer, or optical isomer thereof.

In some embodiments, wherein the compound of formula (I) is selected from the group consisting of compounds 6a-9s:

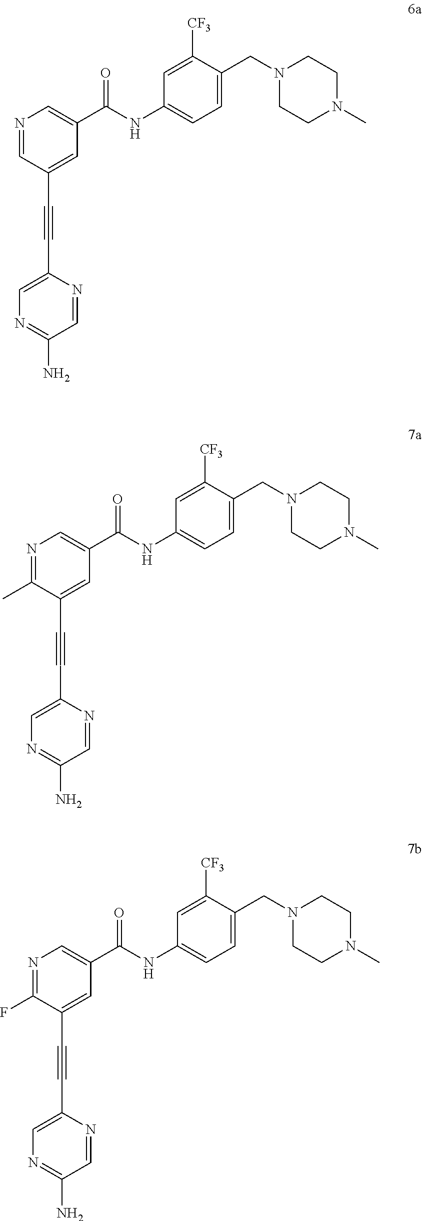

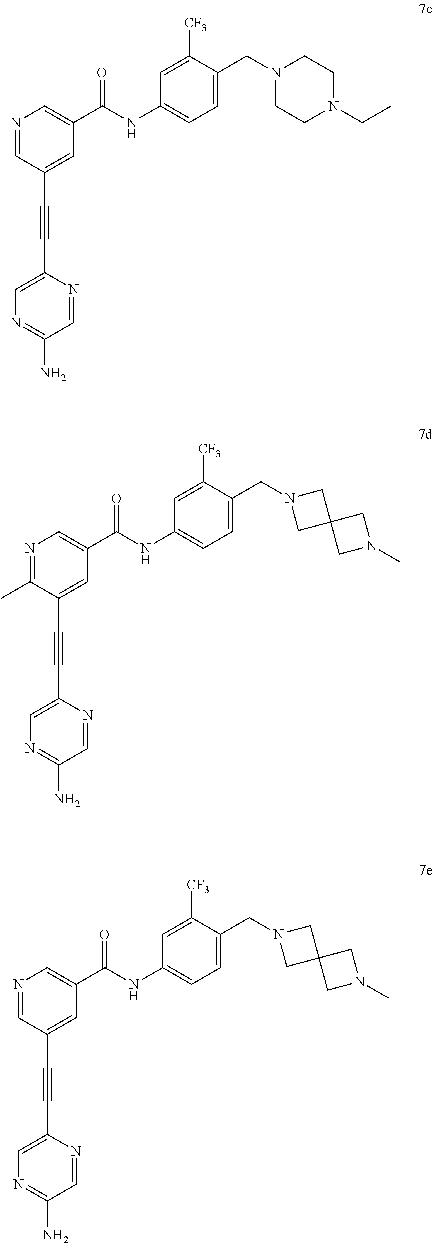

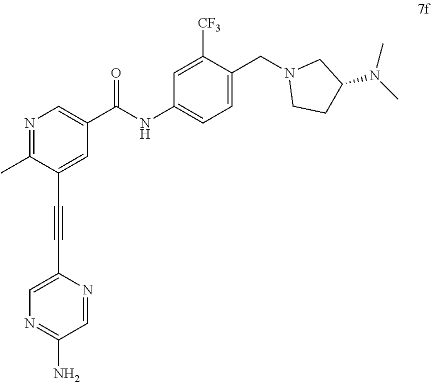

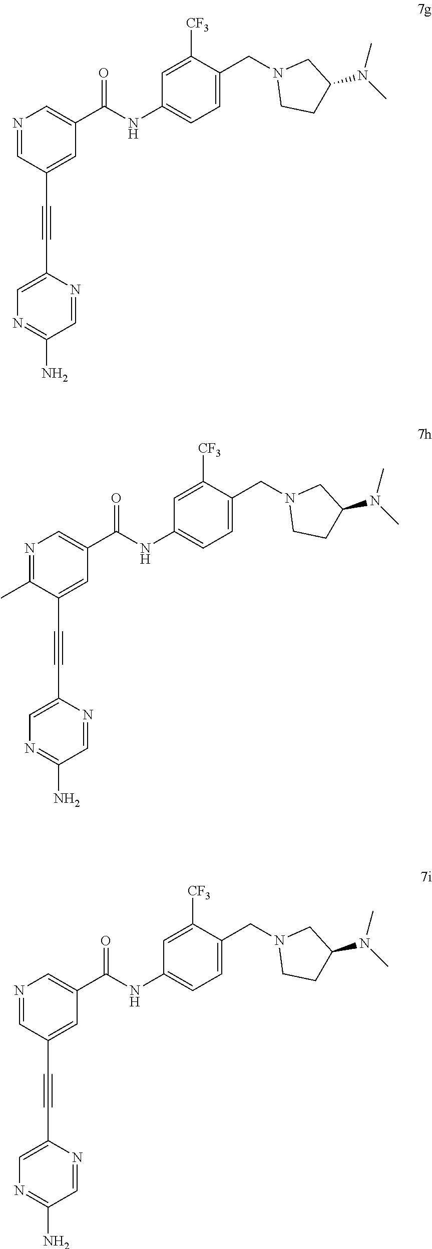

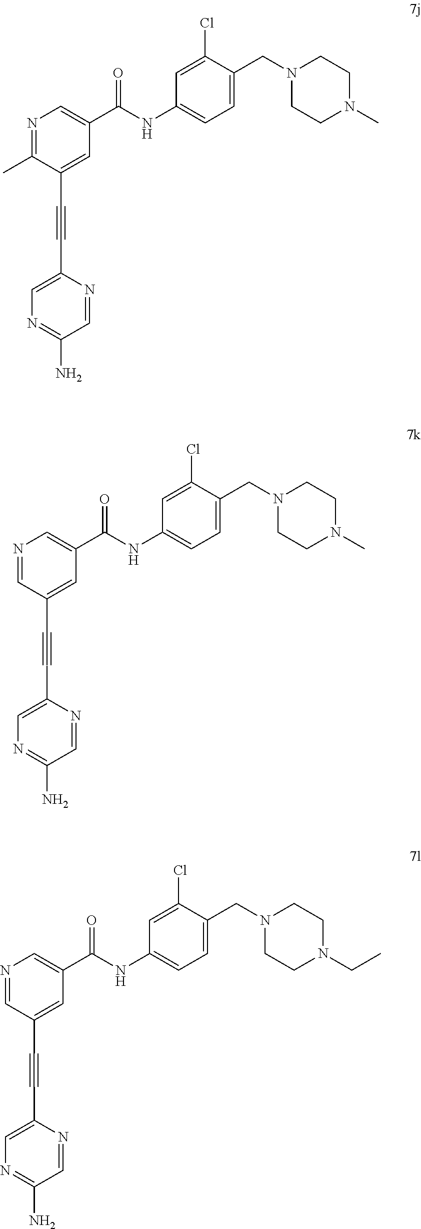

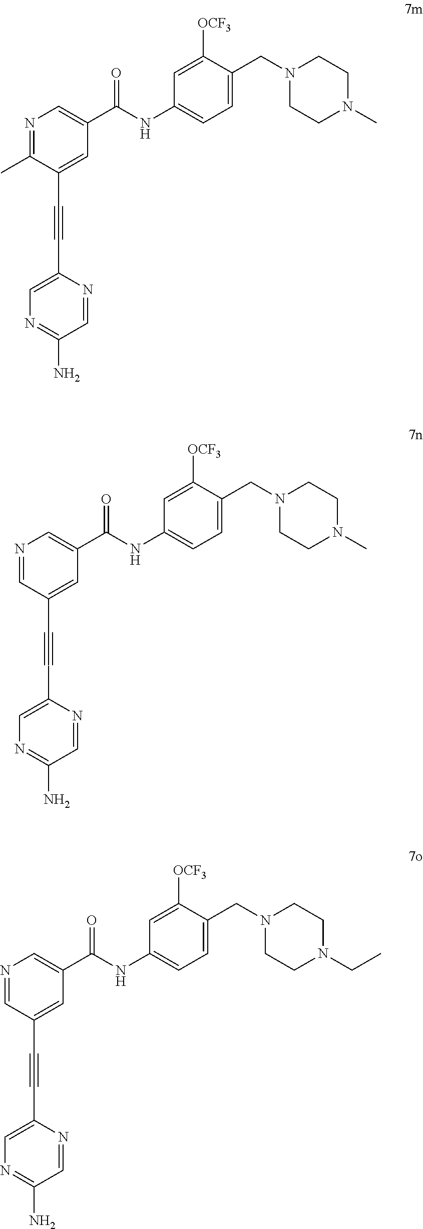

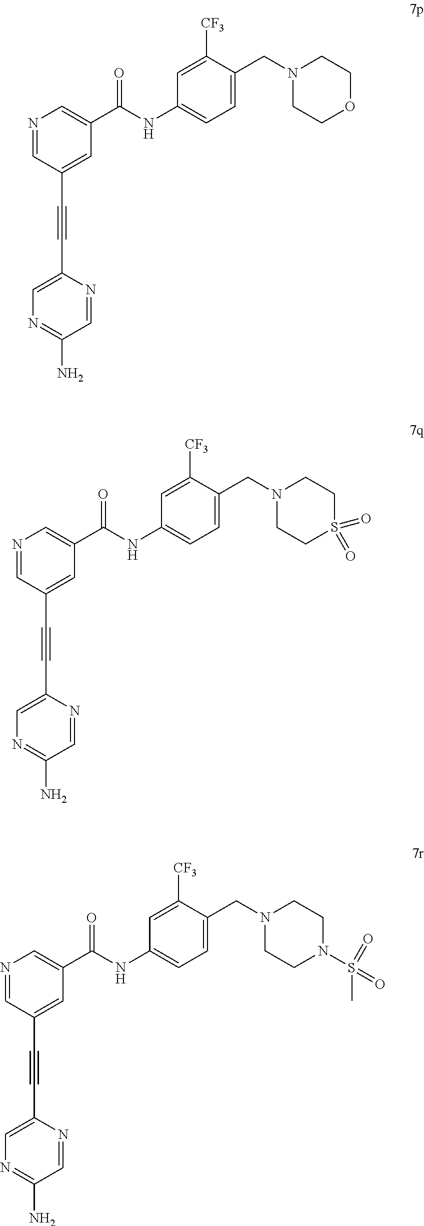 [SMILES: Nc1cnc(C#Cc2cncc(C(=O)Nc3ccc(CN4CCOCC4)c(C(F)(F)F)c3)c2)cn1 ; Nc1cnc(C#Cc2cncc(C(=O)Nc3ccc(CN4CCS(=O)(=O)CC4)c(C(F)(F)F)c3)c2)cn1 ; CS(=O)(=O)N1CCN(Cc2ccc(NC(=O)c3cncc(C#Cc4cnc(N)cn4)c3)cc2C(F)(F)F)CC1]

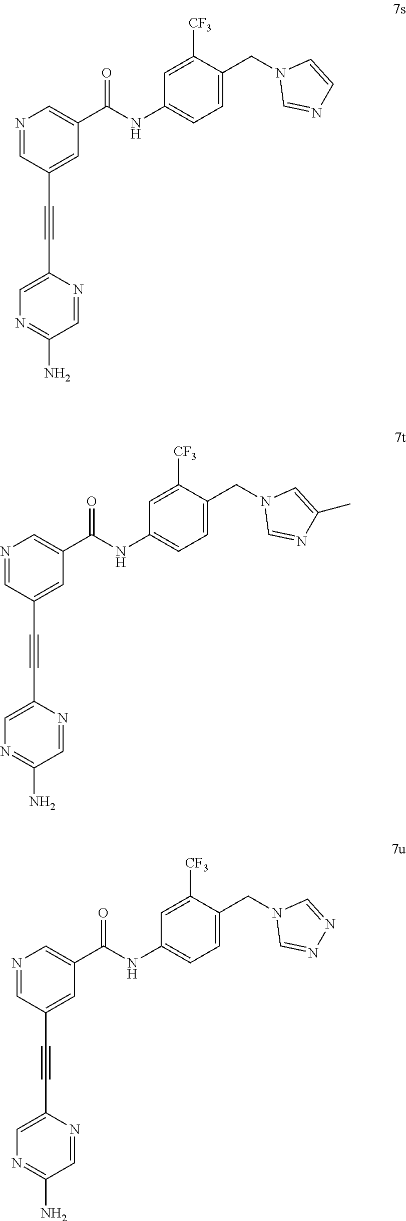 [SMILES: Nc1cnc(C#Cc2cncc(C(=O)Nc3ccc(Cn4ccnc4)c(C(F)(F)F)c3)c2)cn1 ; Cc1cn(Cc2ccc(NC(=O)c3cncc(C#Cc4cnc(N)cn4)c3)cc2C(F)(F)F)cn1 ; Nc1cnc(C#Cc2cncc(C(=O)Nc3ccc(Cn4cnnc4)c(C(F)(F)F)c3)c2)cn1]

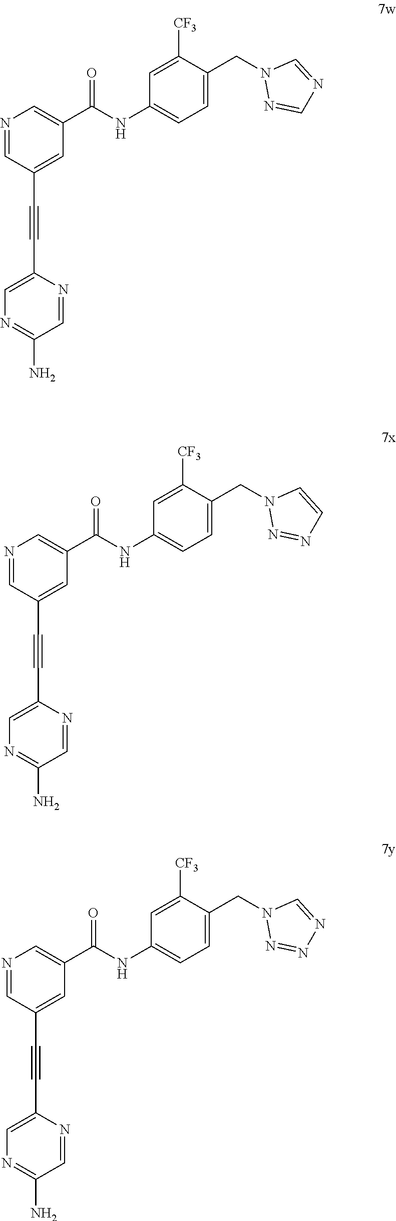 [SMILES: Nc1cnc(C#Cc2cncc(C(=O)Nc3ccc(Cn4cncn4)c(C(F)(F)F)c3)c2)cn1 ; Nc1cnc(C#Cc2cncc(C(=O)Nc3ccc(Cn4ccnn4)c(C(F)(F)F)c3)c2)cn1 ; Nc1cnc(C#Cc2cncc(C(=O)Nc3ccc(Cn4cnnn4)c(C(F)(F)F)c3)c2)cn1]

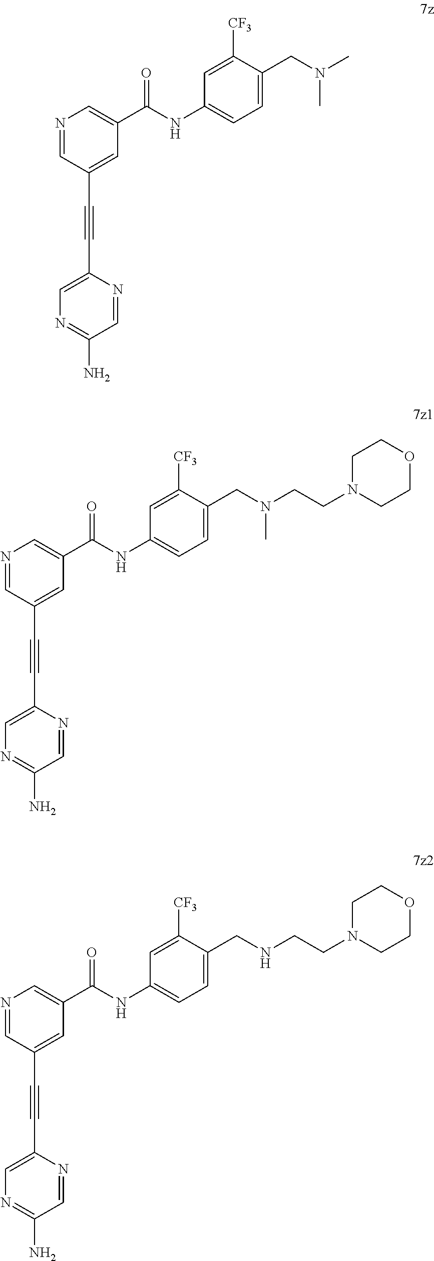 [SMILES: CN(C)Cc1ccc(NC(=O)c2cncc(C#Cc3cnc(N)cn3)c2)cc1C(F)(F)F ; CN(CCN1CCOCC1)Cc1ccc(NC(=O)c2cncc(C#Cc3cnc(N)cn3)c2)cc1C(F)(F)F ; Nc1cnc(C#Cc2cncc(C(=O)Nc3ccc(CNCCN4CCOCC4)c(C(F)(F)F)c3)c2)cn1]

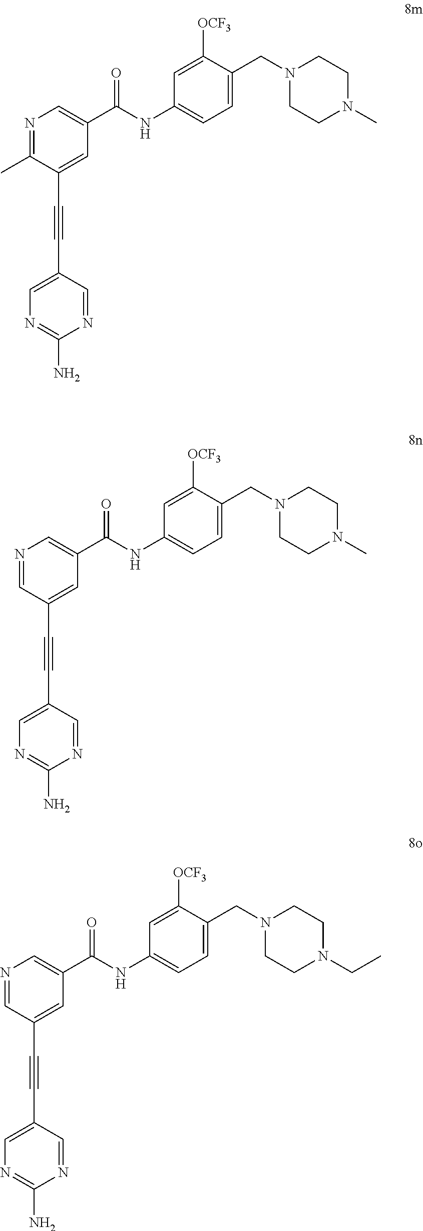

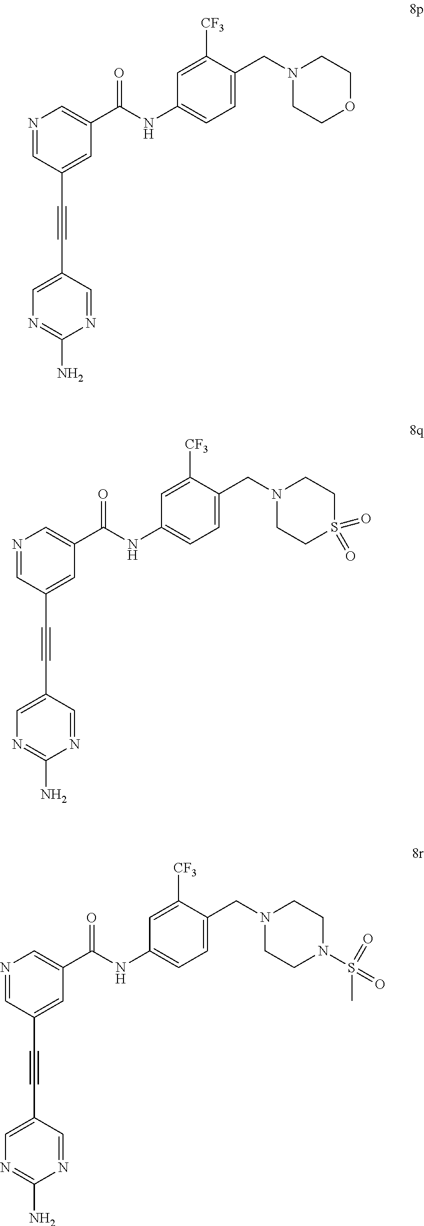 [SMILES: Nc1ncc(C#Cc2cncc(C(=O)Nc3ccc(CN4CCOCC4)c(C(F)(F)F)c3)c2)cn1 ; Nc1ncc(C#Cc2cncc(C(=O)Nc3ccc(CN4CCS(=O)(=O)CC4)c(C(F)(F)F)c3)c2)cn1 ; CS(=O)(=O)N1CCN(Cc2ccc(NC(=O)c3cncc(C#Cc4cnc(N)nc4)c3)cc2C(F)(F)F)CC1]

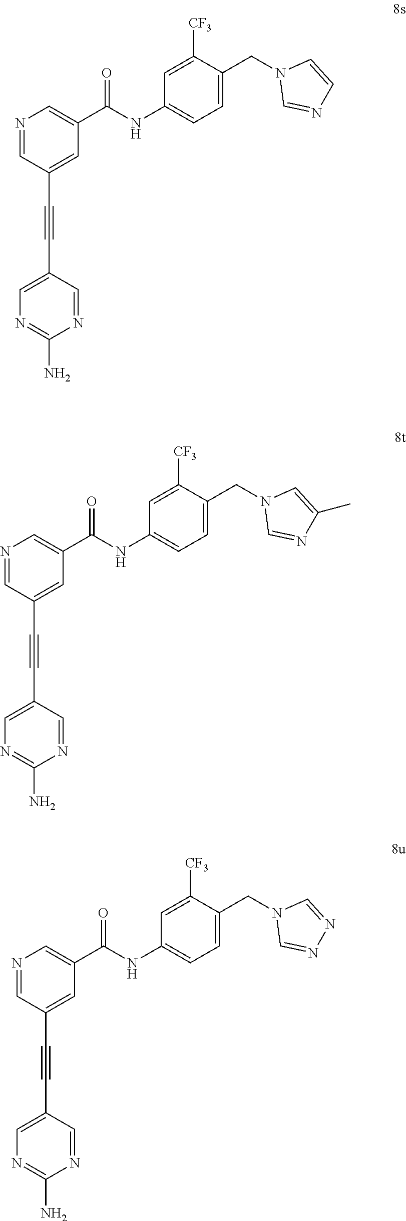 [SMILES: Nc1ncc(C#Cc2cncc(C(=O)Nc3ccc(Cn4ccnc4)c(C(F)(F)F)c3)c2)cn1 ; Cc1cn(Cc2ccc(NC(=O)c3cncc(C#Cc4cnc(N)nc4)c3)cc2C(F)(F)F)cn1 ; Nc1ncc(C#Cc2cncc(C(=O)Nc3ccc(Cn4cnnc4)c(C(F)(F)F)c3)c2)cn1]

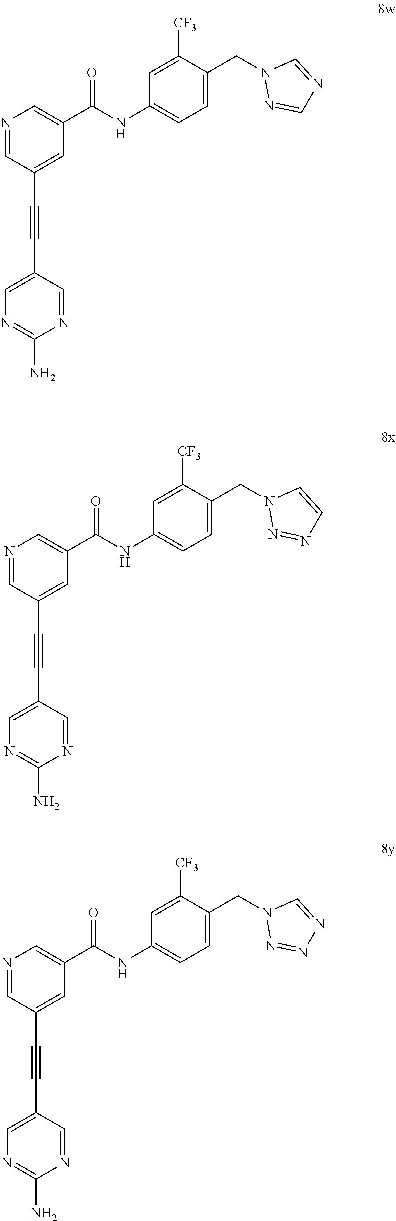 [SMILES: Nc1ncc(C#Cc2cncc(C(=O)Nc3ccc(Cn4cncn4)c(C(F)(F)F)c3)c2)cn1 ; Nc1ncc(C#Cc2cncc(C(=O)Nc3ccc(Cn4ccnn4)c(C(F)(F)F)c3)c2)cn1 ; Nc1ncc(C#Cc2cncc(C(=O)Nc3ccc(Cn4cnnn4)c(C(F)(F)F)c3)c2)cn1]

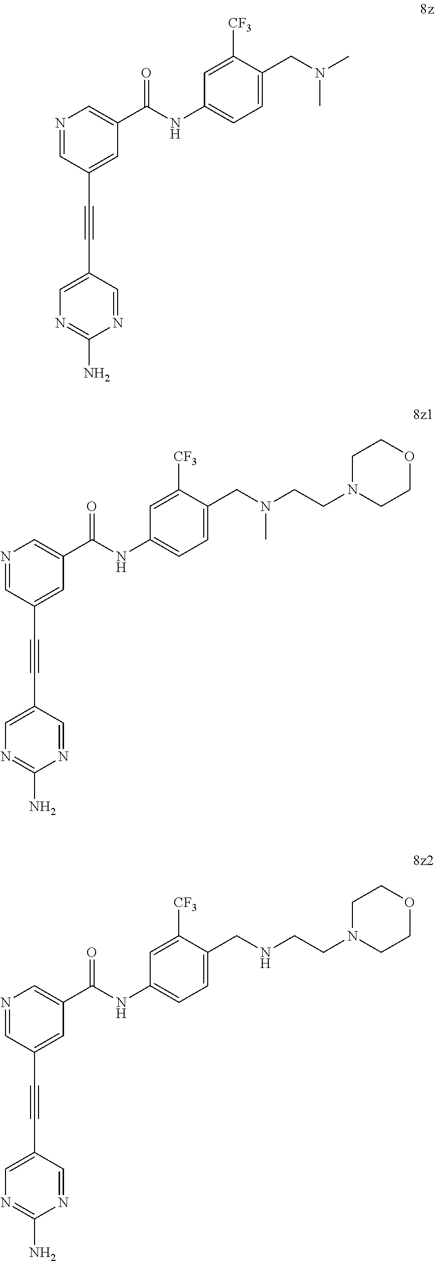 [SMILES: CN(C)Cc1ccc(NC(=O)c2cncc(C#Cc3cnc(N)nc3)c2)cc1C(F)(F)F ; CN(CCN1CCOCC1)Cc1ccc(NC(=O)c2cncc(C#Cc3cnc(N)nc3)c2)cc1C(F)(F)F ; Nc1ncc(C#Cc2cncc(C(=O)Nc3ccc(CNCCN4CCOCC4)c(C(F)(F)F)c3)c2)cn1]

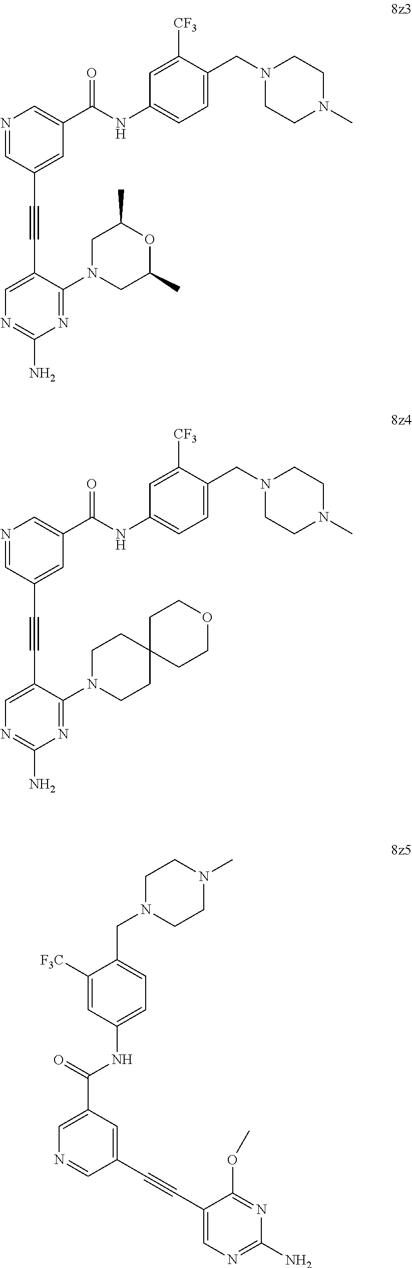 [SMILES: C[C@@H]1CN(c2nc(N)ncc2C#Cc2cncc(C(=O)Nc3ccc(CN4CCN(C)CC4)c(C(F)(F)F)c3)c2)C[C@H](C)O1 ; CN1CCN(Cc2ccc(NC(=O)c3cncc(C#Cc4cnc(N)nc4N4CCC5(CCOCC5)CC4)c3)cc2C(F)(F)F)CC1 ; COc1nc(N)ncc1C#Cc1cncc(C(=O)Nc2ccc(CN3CCN(C)CC3)c(C(F)(F)F)c2)c1]

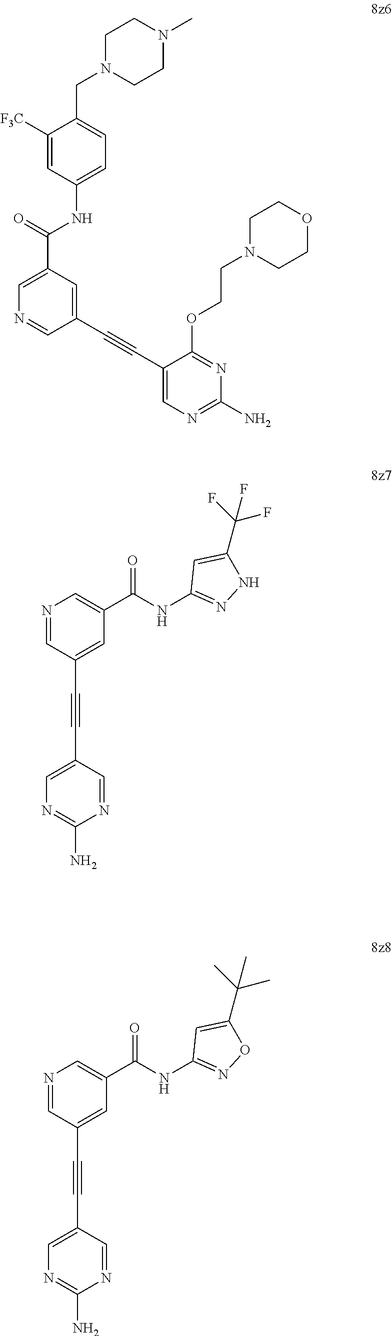 [SMILES: CN1CCN(Cc2ccc(NC(=O)c3cncc(C#Cc4cnc(N)nc4OCCN4CCOCC4)c3)cc2C(F)(F)F)CC1 ; Nc1ncc(C#Cc2cncc(C(=O)Nc3cc(C(F)(F)F)[nH]n3)c2)cn1 ; CC(C)(C)c1cc(NC(=O)c2cncc(C#Cc3cnc(N)nc3)c2)no1]

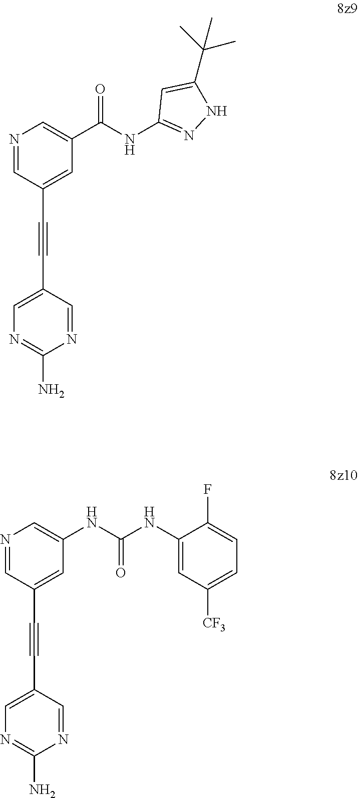 [SMILES: CC(C)(C)c1cc(NC(=O)c2cncc(C#Cc3cnc(N)nc3)c2)n[nH]1 ; Nc1ncc(C#Cc2cncc(NC(=O)Nc3cc(C(F)(F)F)ccc3F)c2)cn1]

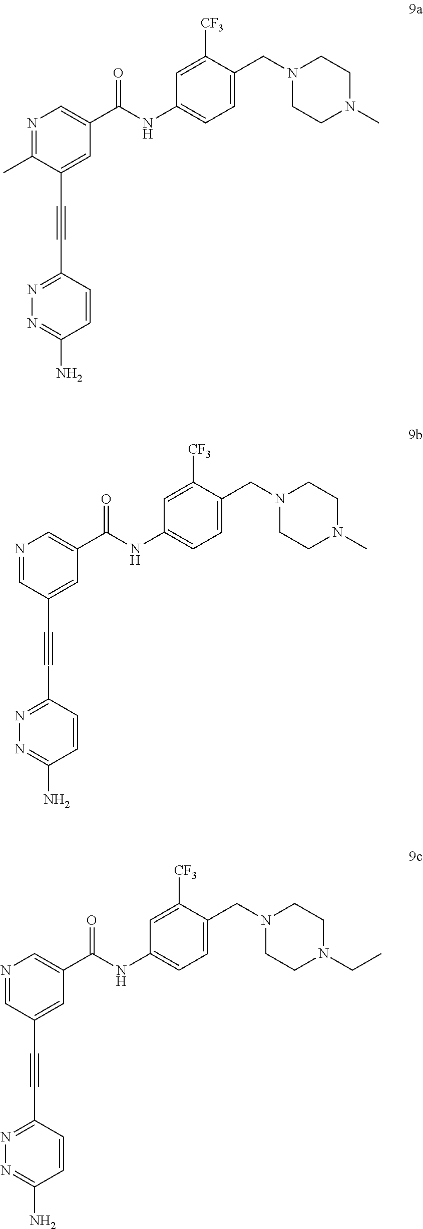

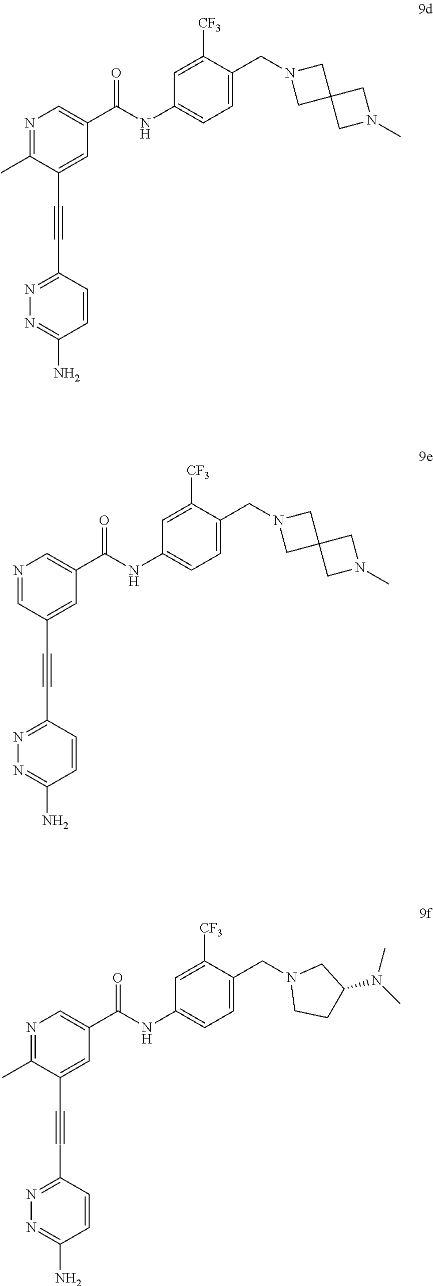

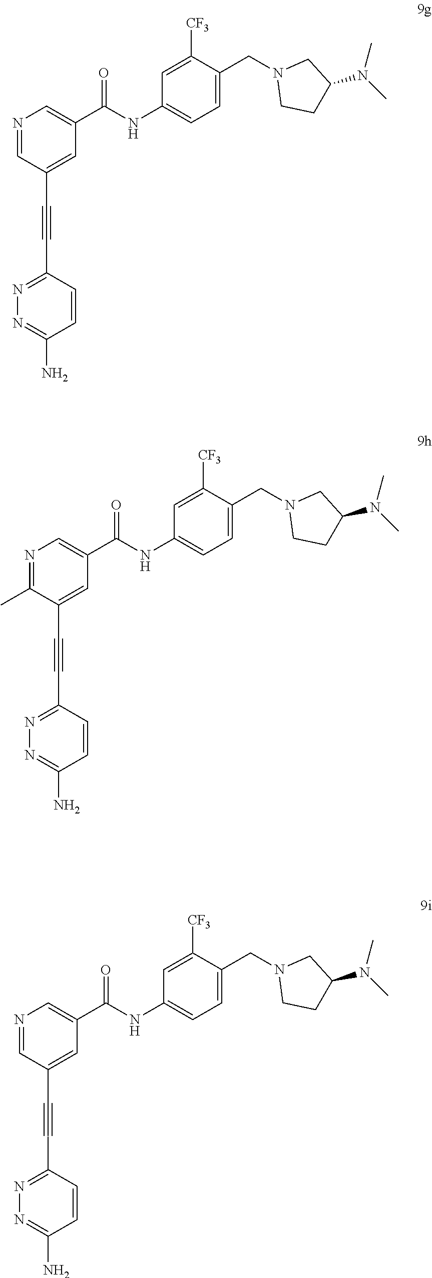

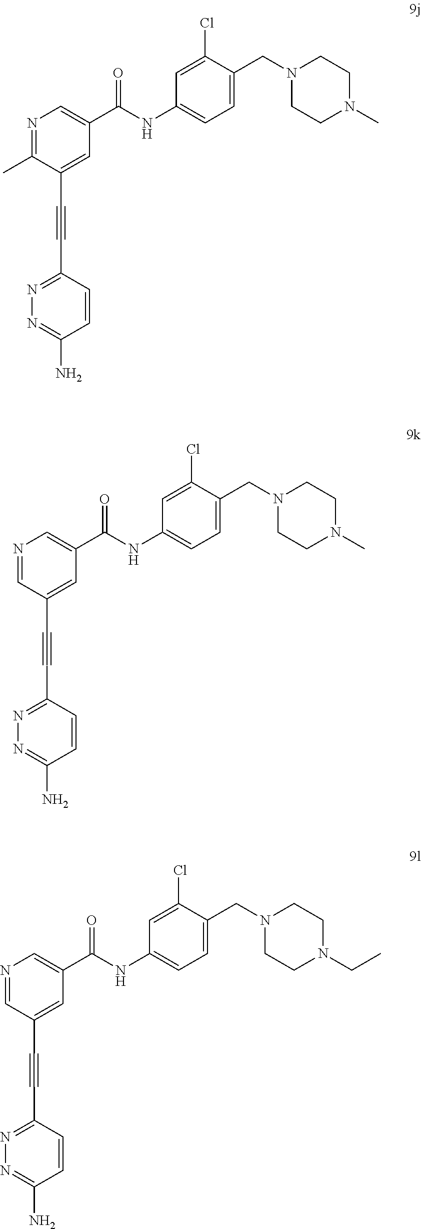

or a pharmaceutically acceptable salt, hydrate, tautomer, or optical isomer thereof.

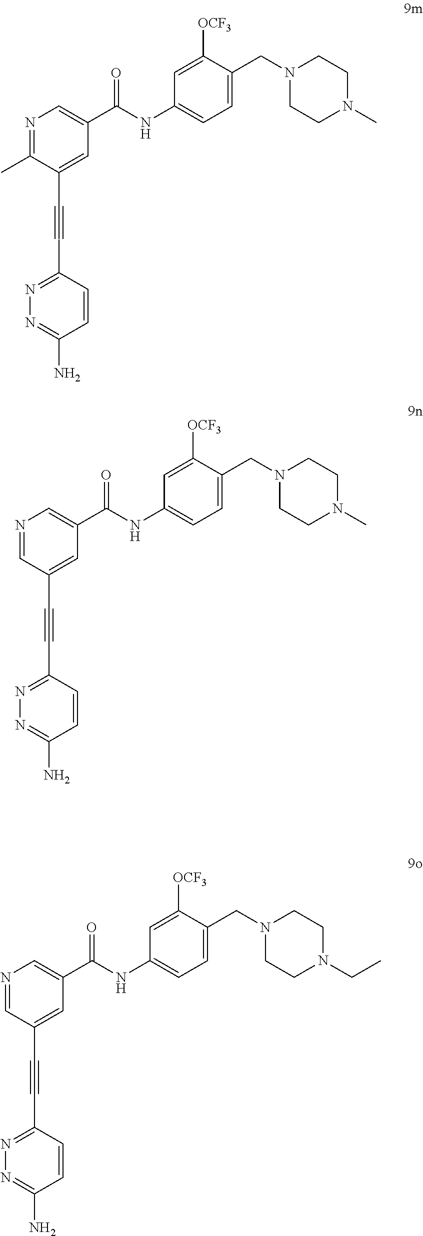

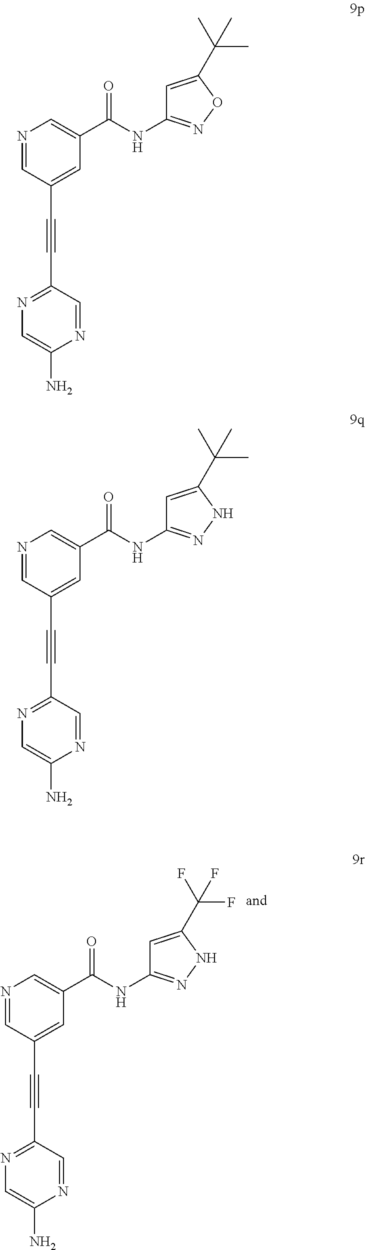 [SMILES: CC(C)(C)c1cc(NC(=O)c2cncc(C#Cc3cnc(N)cn3)c2)no1 ; CC(C)(C)c1cc(NC(=O)c2cncc(C#Cc3cnc(N)cn3)c2)n[nH]1 ; Nc1cnc(C#Cc2cncc(C(=O)Nc3cc(C(F)(F)F)[nH]n3)c2)cn1]

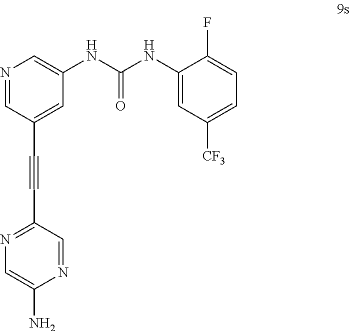 [SMILES: Nc1cnc(C#Cc2cncc(NC(=O)Nc3cc(C(F)(F)F)ccc3F)c2)cn1]

Further provided is a compound of formula (IA), or a pharmaceutically acceptable salt, hydrate, tautomer, or optical isomer thereof:

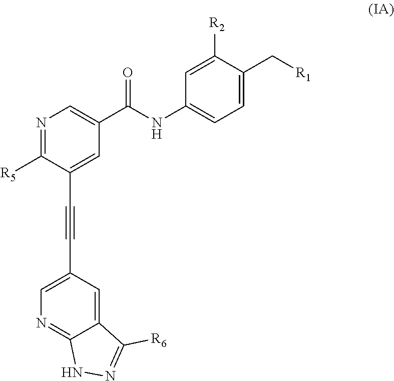

wherein
R₁ is NRₐR_{b}, wherein Rₐ and R_{b} each independently represent a group selected from H, alkyl, cycloalkyl, heterocycloalkyl, aryl, heteroaryl, —(CH₂)ₚ-alkyl, —(CH₂)ₚ-cycloalkyl, —(CH₂)ₚ-heterocycloalkyl, —(CH₂)ₚ—SO₂R_{c}, —(CH₂)ₚ—OR_{c}, and —(CH₂)ₚ—NRₐR_{b}, wherein Rₐ and R_{b} each independently is optionally substituted with at least one of alkyl, halo, OR_{c}, and NH₂; or
Rₐ and R_{b}, together with the nitrogen atom to which they are attached, form a 4- to 8-membered heterocycloalkyl group, which is a mono or a bicyclic ring, either of which is optionally substituted with a group selected from alkyl, cycloalkyl, halo, nitro, CN, SO₂R_{c}, OR_{d}, —(CH₂)ₚ—OR_{c} and NR_{c}R_{c};
R_{c} is H or alkyl;
R_{d} is H, halo, OH, CN, alkyl, —(CH₂)ₚ-alkyl, —(CH₂)ₚ-cycloalkyl, —(CH₂)ₚ-heterocycloalkyl, —(CH₂)ₚ—SO₂R_{d}, —(CH₂)ₚ—OR_{d}. —(CH₂)ₚ—(OR_{d})₂, or —(CH₂)ₚ—NRₐR_{b};
p is 0-5;
R₂ represents a group independently selected from alkyl, CF₃, OCF₃, and halo;
R₅ represents a group independently selected from H, alkyl, and halo;
R₆ represents a group independently selected from H, alkyl, ORₐ, and NRₐR_{b}, wherein Rₐ and R_{b} each independently represent a group selected from H, alkyl, cycloalkyl, heterocycloalkyl, aryl, heteroaryl, —(CH₂)ₚ-alkyl, —(CH₂)ₚ-cycloalkyl, —(CH₂)ₚ-heterocycloalkyl, —(CH₂)ₚ—SO₂Re, —(CH₂)ₚ—OR_{c}, and —(CH₂)ₚ—NRₐR_{b}, wherein Rₐ and R_{b} each independently is optionally substituted with at least one of alkyl, halo, OR_{c}, and NH₂; or
Rₐ and R_{b}, together with the nitrogen atom to which they are attached, form a 4- to 8-membered heterocycloalkyl group, which is a mono or a bicyclic ring, either of which is optionally substituted with a group selected from alkyl, cycloalkyl, halo, nitro, CN, SO₂Re, OR_{d}, —(CH₂)ₚ—OR_{c}, and NR_{c}R_{c}, wherein R_{c}, R_{d} and p are as defined above.

In some embodiments, R₆ represents a group selected from:

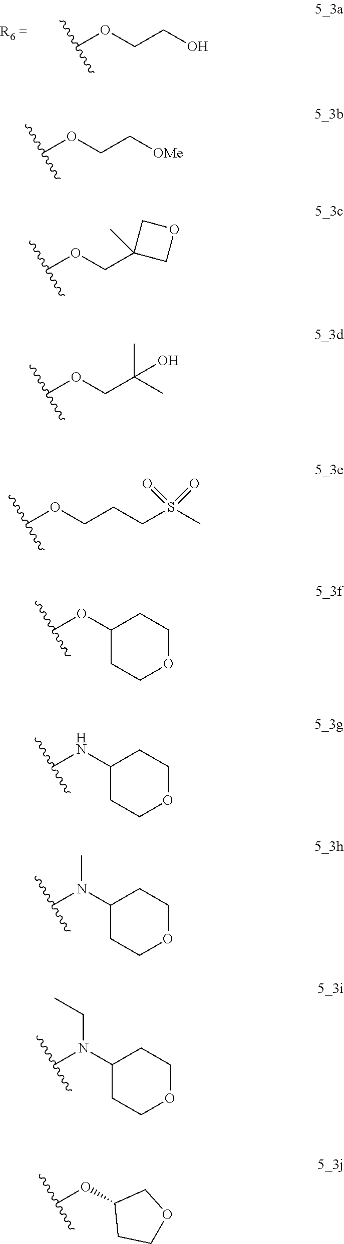

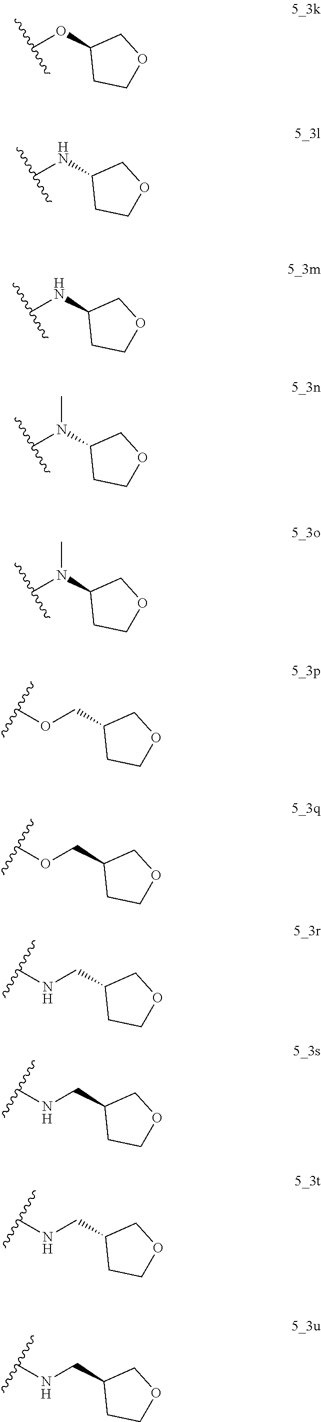

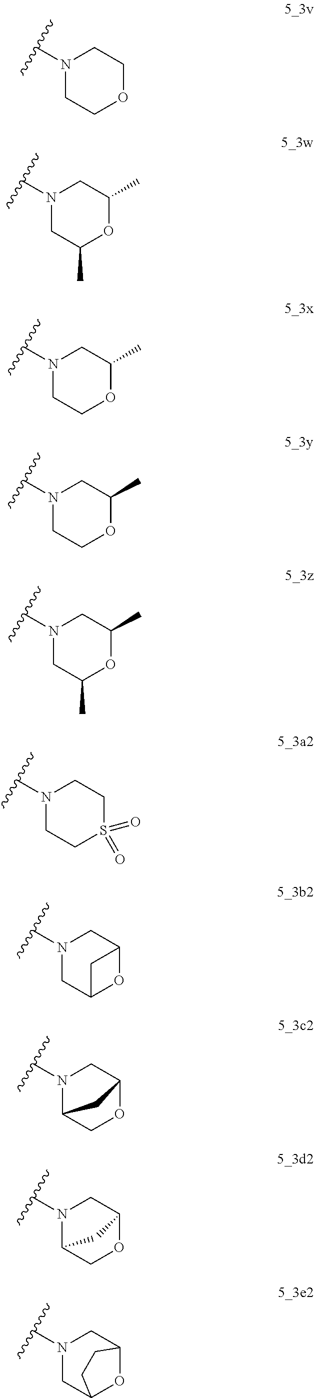

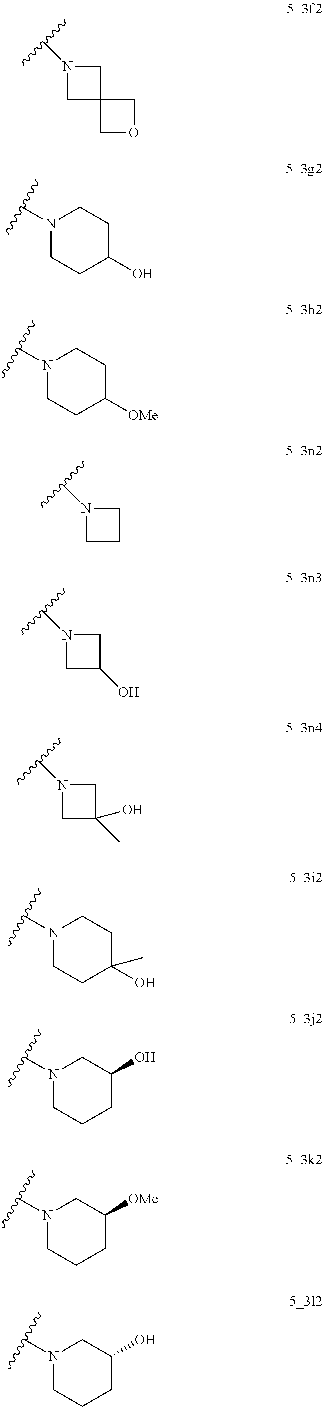

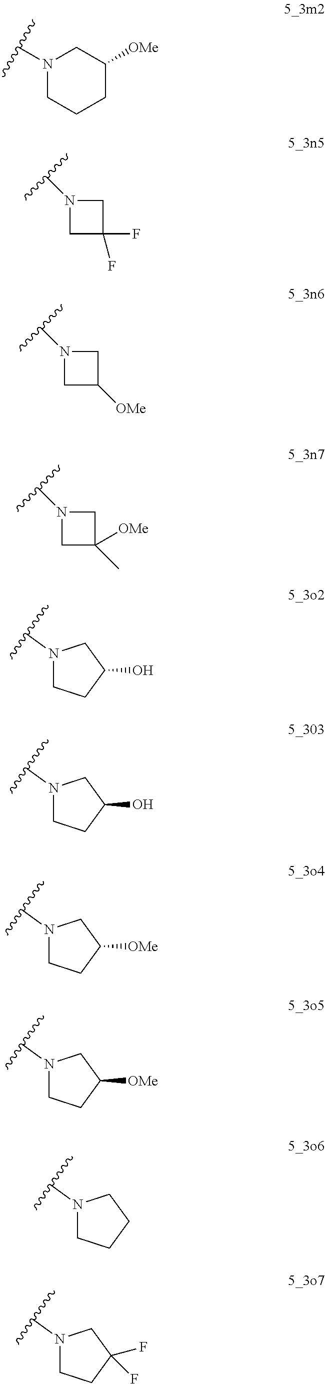

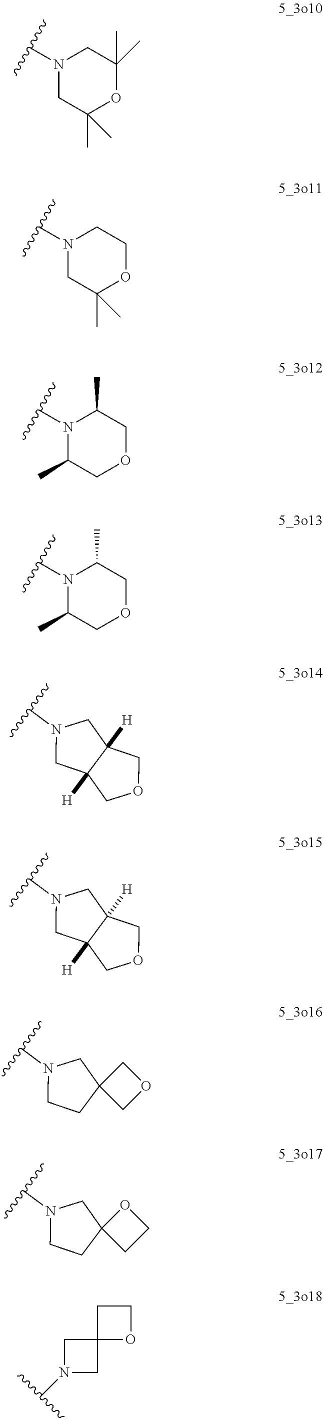

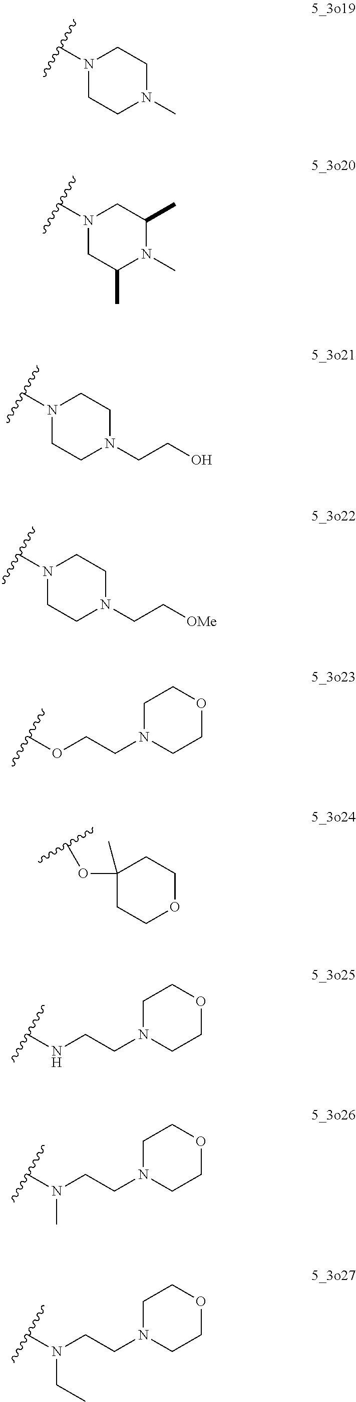

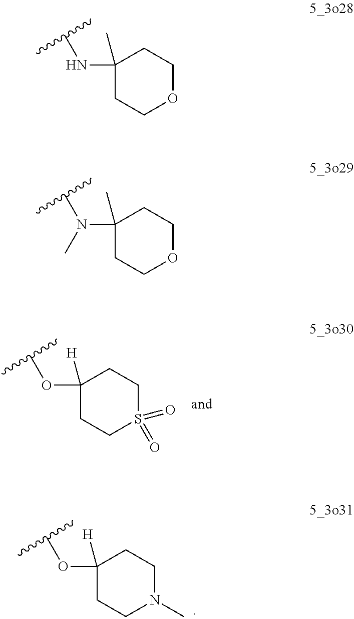

In some embodiments, the compound of formula (IA) is 5-((1H-pyrazolo[3,4-b]pyridin-5-yl)ethynyl)-N-(4-((4-methylpiperazin-1-yl)methyl)-3-(trifluoromethyl)phenyl)nicotinamide,

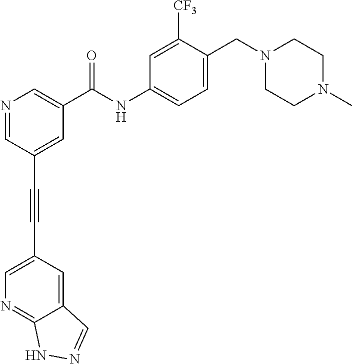 [SMILES: CN1CCN(Cc2ccc(NC(=O)c3cncc(C#Cc4cnc5[nH]ncc5c4)c3)cc2C(F)(F)F)CC1]

or a pharmaceutically acceptable salt, hydrate, tautomer, or optical isomer thereof.

In some embodiments, the compound of formula (IA) is 5-((3-methyl-1H-pyrazolo[3,4-b]pyridin-5-yl)ethynyl)-N-(4-((4-methylpiperazin-1-yl)methyl)-3-(trifluoromethyl)phenyl)nicotinamide,

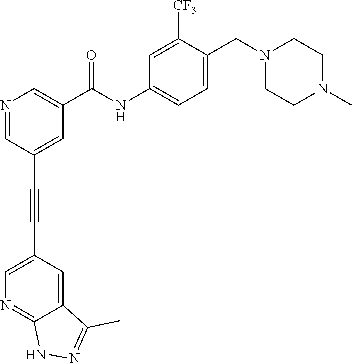 [SMILES: Cc1n[nH]c2ncc(C#Cc3cncc(C(=O)Nc4ccc(CN5CCN(C)CC5)c(C(F)(F)F)c4)c3)cc12]

or a pharmaceutically acceptable salt, hydrate, tautomer, or optical isomer thereof.

In some embodiments, the compound of formula (IA) is selected from the group consisting of compounds 10a-10h:

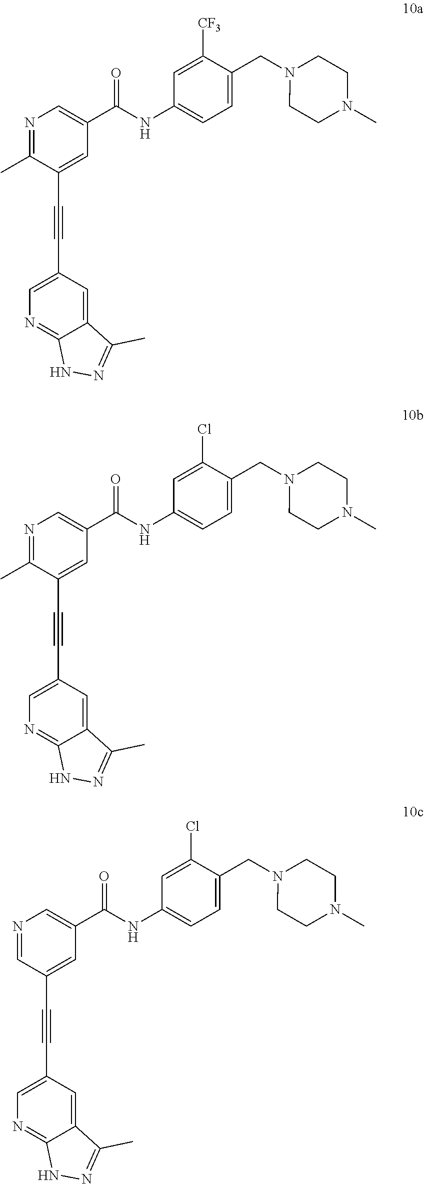

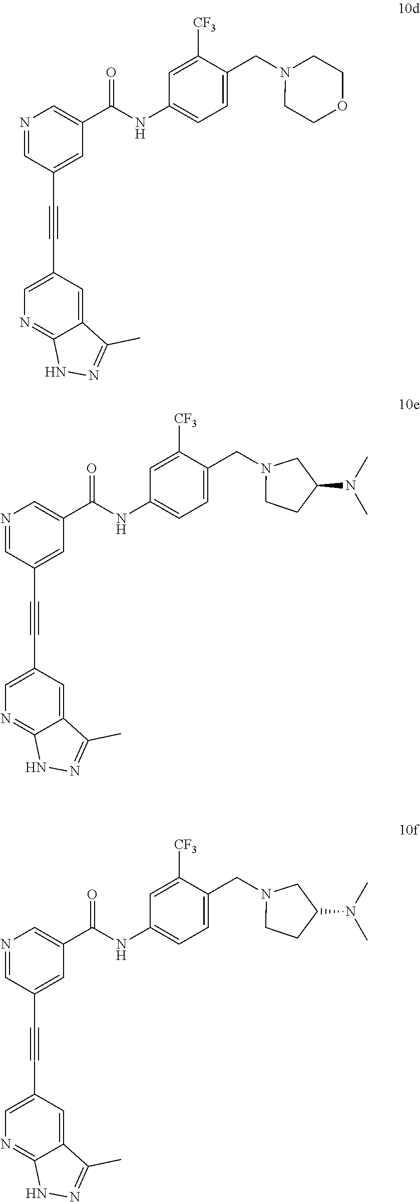

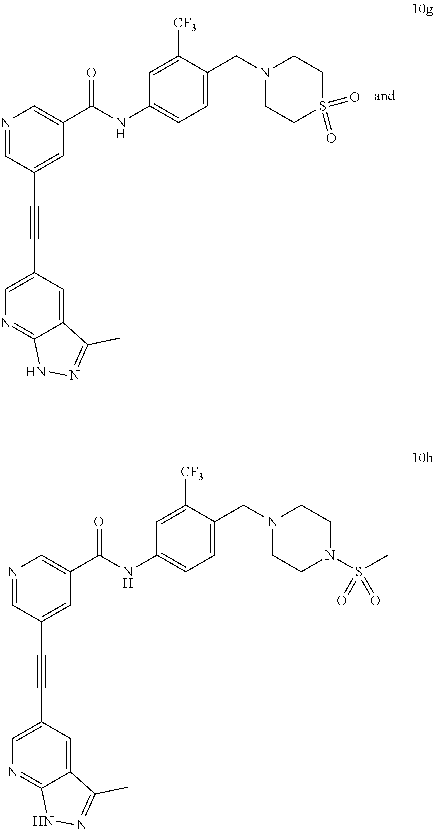 [SMILES: Cc1n[nH]c2ncc(C#Cc3cncc(C(=O)Nc4ccc(CN5CCS(=O)(=O)CC5)c(C(F)(F)F)c4)c3)cc12 ; Cc1n[nH]c2ncc(C#Cc3cncc(C(=O)Nc4ccc(CN5CCN(S(C)(=O)=O)CC5)c(C(F)(F)F)c4)c3)cc12]

or a pharmaceutically acceptable salt, hydrate, tautomer, or optical isomer thereof.

The compound of formula (I) or (IA), or a pharmaceutically acceptable salt hydrate, tautomer, or optical isomer of either of the foregoing selectively inhibits RET kinase mutants. In exemplary embodiments, RET kinase mutants are G810C, G810A, G810R, G810S, and V804M.

Provided is a pharmaceutical composition comprising a compound of formula (I) or a pharmaceutically acceptable salt, hydrate, tautomer, or optical isomer thereof and a pharmaceutically acceptable carrier, excipient, or diluent.

Provided is a pharmaceutical composition comprising a compound of formula (IA) or a pharmaceutically acceptable salt, hydrate, tautomer, or optical isomer thereof, and a pharmaceutically acceptable carrier, excipient, or diluent.

Further provided is a method of treating or inhibiting a rearranged during transfection (RET) kinase-altered cancer, wherein the method comprises inhibiting RET kinase mutants by administering to a patient in need thereof a therapeutically effective amount of a compound of formula (I) or a pharmaceutically acceptable salt, hydrate, tautomer or optical isomer thereof, optionally as a pharmaceutical composition comprising the compound and a pharmaceutically acceptable carrier, excipient, or diluent. The compound of formula (I) can be administered orally. In some embodiments, RET mutant is G810.

Further provided is a method of treating or inhibiting RET kinase-altered cancer, wherein the method comprises inhibiting RET kinase mutants by administering to a patient in need thereof a therapeutically effective amount of a compound of formula (IA) or a pharmaceutically acceptable salt, hydrate, tautomer, or optical isomer thereof, optionally as a pharmaceutical composition comprising the compound and a pharmaceutically acceptable carrier, excipient, or diluent. The compound of formula (IA) can be administered orally. In some embodiments, RET mutant is G810.

In some embodiments, the compound of formula (I) or (IA) can be administered orally. In some embodiments, examples of RET kinase-altered cancers include, but are not limited to, lung cancer, thyroid cancer, pancreatic cancer, and breast cancer.

Further provided is a method of inhibiting a RET kinase mutants in a patient, which method comprises administering to the patient in need thereof a therapeutically effective amount of the compound of formula (I) or (IA) or a pharmaceutically acceptable salt, hydrate, tautomer or optical isomer of either of the foregoing, or a pharmaceutical composition comprising the same and a pharmaceutically acceptable carrier, excipient, or diluent, whereupon RET mutants in the patient is inhibited. In some embodiments, the patient has RET-kinase altered cancer.

### BRIEF DESCRIPTION OF THE DRAWINGS

The above and other objects, features, and advantages of the present disclosure will be apparent when the description is read in conjunction with the drawings.

FIG. 1 shows in vivo activity of alkynyl nicotinamide-based rearranged during transfection (RET) protein tyrosine kinase inhibitors (RET-TKIs) on B/KR(G810C) CDX tumors. Mice bearing B/KR(G810C) CDX tumors were treated with the test compounds at the indicated doses by daily oral gavage. Tumor dimensions were measured with a caliper, and tumor sizes were estimated using the Formula: tumor size=(Length×width×width)/2. The data shown are the mean±sd.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of the claimed invention is thereby intended.

The present disclosure is predicated, at least in part, on the discovery in circulating tumor DNA, after an initial response to selpercatinib in a patient with rearranged during transfection (RET) kinase non-small cell lung cancer (NSCLC), of RET G810R, G810S, and G810C. mutations in the RET kinase solvent front before the emergence of clinical resistance. Studies revealed that the intratumor and intertumor heterogeneity with distinct disease subclones containing G810S. G810R, and G810C mutations in multiple disease sites indicate convergent evolution on the G810 residue resulting in a common mechanism of resistance. RET kinase G810 solvent-front mutations represent the first described recurrent mechanism of resistance to selective RET kinase inhibition with selpercatinib (Solomon et at., Journal of Thoracic Oncology, 2020, 15(4), 541-549).

Pralsetinib and selpercatinib inhibit RET kinase by binding both the front and the back pockets of the active site of RET kinase, but do not go through the gate region and instead wrap around it. This means that the gatekeeper mutations in resistant cancers would have no effect on pralsetinib and selpercatinib. These inhibitors interact with several non-essential amino acid residues of the RET kinase and, hence, are vulnerable to drug-resistant mutations in these amino acids. The strongest resistance to both drugs was seen to be conferred by substitutions of the glycine 810 residue in the solvent front region, which implies that the RET kinase with glycine 810 mutations will be least inhibited by these drugs and particularly important for causing acquired resistance.

For the purpose of discussion, mutations are discussed with reference to the proto-oncogene tyrosine-protein kinase receptor Ret, i.e., RET kinase, protein sequence (UniProt ID P07949, GenPept ID: NP_066124).

[SEQ ID NO: 1]
MAKATSGAAG LRLLLLLLLP LLGKVALGLY FSRDAYWEKL YVDQAAGTPL LYVHALRDAP
EEVPSFRLGQ HLYGTYRTRL HENNWICIQE DTGLLYLNRS LDHSSWEKLS VRNRGFPLLT
VYLKVFLSPT SLREGECQWP GCARVYFSFF NTSFPACSSL KPRELCFPET RPSFRIRENR
PPGTFHQFRL LPVQFLCPNI SVAYRLLEGE GLPFRCAPDS LEVSTRWALD REQREKYELV
AVCTVHAGAR EEVVMVPFPV TVYDEDDSAP TFPAGVDTAS AVVEFKRKED TVVATLRVFD
ADVVPASGEL VRRYTSTLLP GDTWAQQTFR VEHWPNETSV QANGSFVRAT VHDYRLVLNR
NLSISENRTM QLAVLVNDSD FQGPGAGVLL LHFNVSVLPV SLHLPSTYSL SVSRRARRFA
QIGKVCVENC QAFSGINVOY KLHSSGANCS TLGVVTSAED TSGILFVNDT KALRRPKCAE
LHYMVVATDQ QTSRQAQAQL LVTVEGSYVA EEAGCPLSCA VSKRRLECEE CGGLGSPTGR
CEWRQGDGKG ITRNFSTCSP STKTCPDGHC DVVETQDINI CPQDCLRGSI VGGHEPGEPR
GIKAGYGTCN CFPEEEKCFC EPEDIQDPLC DELCRTVIAA AVLFSFIVSV LLSAFCIHCY
HKFAHKPPIS SAEMTFRRPA QAFPVSYSSS GARRPSLDSM ENQVSVDAFK ILEDPKWEFP
RKNLVLGKTL GEGEFGKVVK ATAFHLKGRA GYTTVAVKML KENASPSELR DLLSEFNVLK
QVNHPHVIKL YGACSQDGPL LLIVEYAKYG SLRGFLRESR KVGPGYLGSG GSRNSSSLDH
PDERALTMGD LISFAWQISQ GMQYLAEMKL VHRDLAARNI LVAEGRKMKI SDFGLSRDVY
EEDSYVKRSQ GRIPVKWMAI ESLFDHIYTT QSDVWSFGVL LWEIVTLGGN PYPGIPPERL
FNLLKTGHRM ERPDNCSEEM YRLMLQCWKQ EPDKRPVFAD ISKDLEKMMV KRRDYLDLAA
STPSDSLIYD DGLSEEETPL VDCNNAPLPR ALPSTWIENK LYGMSDPNWP GESPVPLTRA
DGTNTGFPRY PNDSVYANWM LSPSAAKLMD TFDS

RET kinase G810 mutations occur by substitutions of the gly cine 810 residue. In the G810C mutation, glycine is replaced with cysteine; in G810R mutation, glycine is replaced with arginine; in G810S mutation, glycine is replaced with serine; in G810A mutation, glycine is replaced with alanine; and in G810V mutation, glycine is replaced with valine. These mutations represent single nucleotide substitutions of the RET G180 codon. Two nucleotide substitutions can also occur, resulting in missense mutations of G810 to other amino acid residues. These mutations are present in the solvent front region of RET Kinase.

The term “G810 solvent front mutations” refers to mutations of glycine 810 residue in a solvent front region. These mutations are specifically located at the floor of the solvent front. The ATP solvent exposed area is called the solvent-front region.

The term “RET kinase-altered” cancer refers to a cancer comprising a RET kinase alteration as described herein.

In view of the above, the present disclosure provides alkynyl nicotinamide compounds that are RET kinase inhibitors, which inhibit RET kinase mutants resistant to RET protein tyrosine kinase inhibitors (RET-TKIs).

RET-TKIs include well-known compounds. Examples of RET-TKIs include, but are not limited to, selpercatinib and pralsetinib.

Thus, provided is a compound of formula (I), or a pharmaceutically acceptable salt, hydrate, tautomer, or optical isomer thereof:

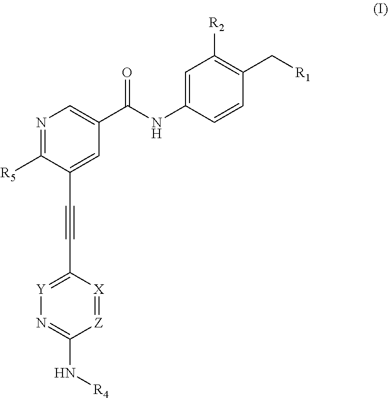

wherein
R₁ is NRₐR_{b}, wherein Rₐ and R_{b} each independently represent a group selected from H, alkyl, cycloalkyl, heterocycloalkyl, aryl, heteroaryl, —(CH₂)ₚ-alkyl, —(CH₂)ₚ-cycloalkyl, —(CH₂)ₚ-heterocycloalkyl, —(CH₂)ₚ—SO₂Re, —(CH₂)ₚ—OR_{c}, and —(CH₂)ₚ—NRₐR_{b}, wherein Rₐ and R_{b} each independently is optionally substituted with at least one of alkyl, halo, OR₀, and NH₂; or
Rₐ and R_{b}, together with the nitrogen atom to which they are attached, form a 4- to 8-membered heterocycloalkyl group, which is a mono or a bicyclic ring, either of which is optionally substituted with a group selected from alkyl, cycloalkyl, halo, nitro, CN, SO₂R_{c}, OR_{d}, —(CH₂)ₚ—OR_{c} and NR_{c}R_{c};
R_{c} is H or alkyl;
R_{d} is H, halo, OH, CN, alkyl, —(CH₂)ₚ-alkyl, —(CH₂)ₚ-cycloalkyl, —(CH₂)ₚ-heterocycloalkyl, —(CH₂)ₚ—SO₂R_{d}. —(CH₂)ₚ—OR_{d}. —(CH₂)ₚ—(OR_{d})₂, or —(CH₂)ₚ—NRₐR_{b};
p is 0-5;
R₂ represents a group independently selected from alkyl, CF₃, OCF₃, and halo;
X, Y, and Z each independently represent a group selected from CR₃ and N with the proviso that the ring of which X, Y, and Z is comprised contains at least two N atoms, wherein R₃ is H, alkyl, ORₐ, or NRₐR_{b}, wherein Rₐ and R_{b} are as defined above;
R₄ represents a group selected from H, halo, OH, CN, alkyl, —(CH₂)ₚ-alkyl, —(CH₂)ₚ-cycloalkyl, —(CH₂)ₚ-heterocycloalkyl, —(CH₂)ₚ—SO₂Rₐ, —(CH₂)ₚ—OR_{d}, —(CH₂)ₚ—(ORₐ)₂, and —(CH₂)ₚ—NRₐR_{b}, wherein Rₐ, R_{b}, R_{d}, and p are as defined above; and
R₅ represents a group independently selected from H, alkyl, and halo.

In some embodiments, each X, Y, and Z is independently a group selected from CR₃ and N with the proviso that the ring of which X, Y, and Z is comprised contains at least two N atoms, wherein R₃ represents a group selected from:

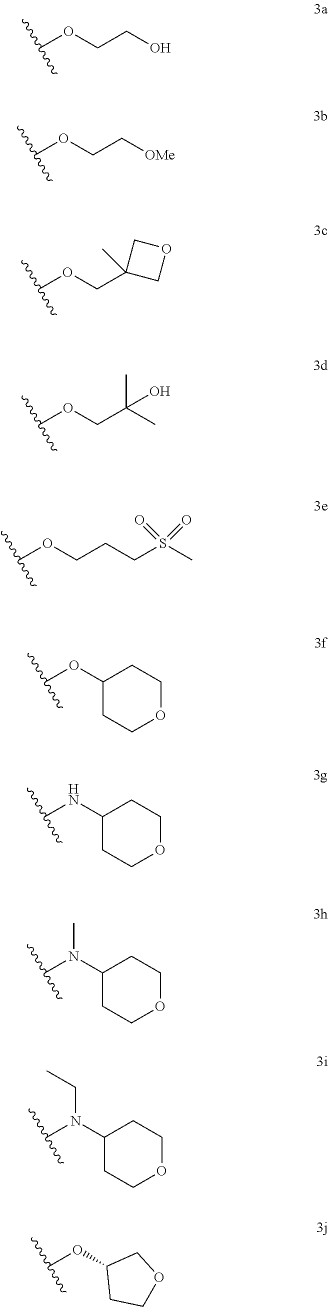

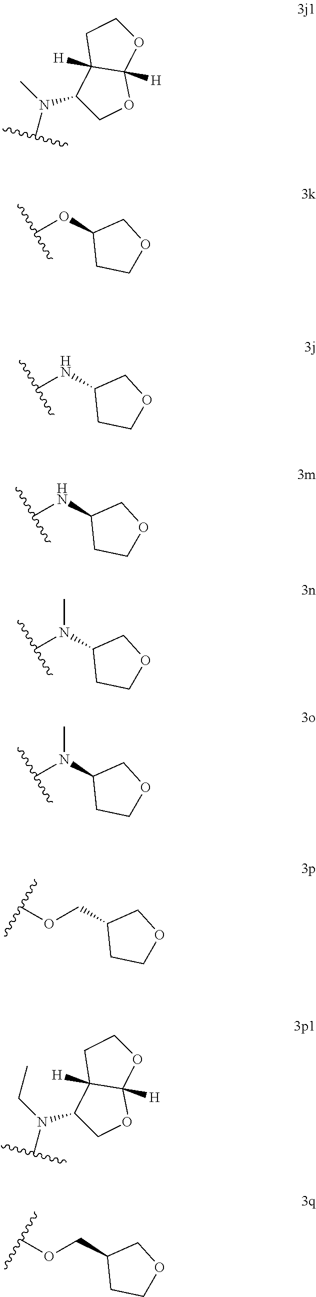

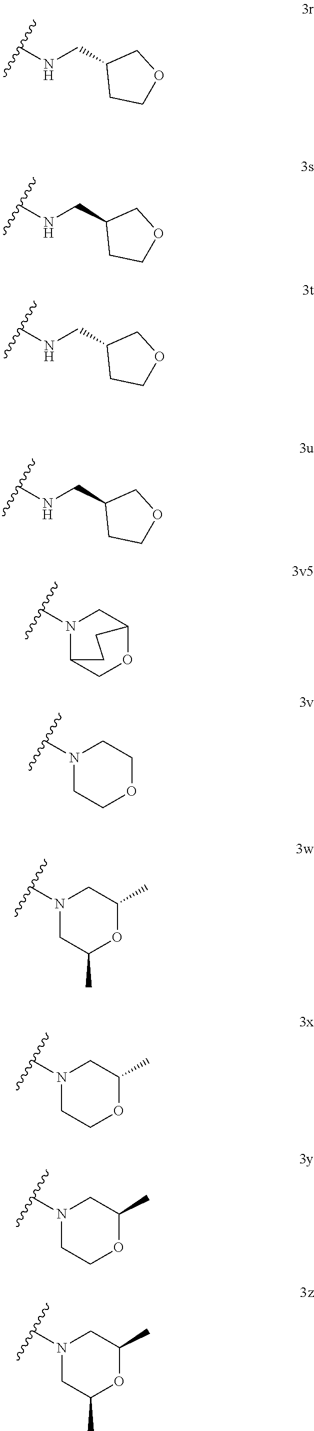

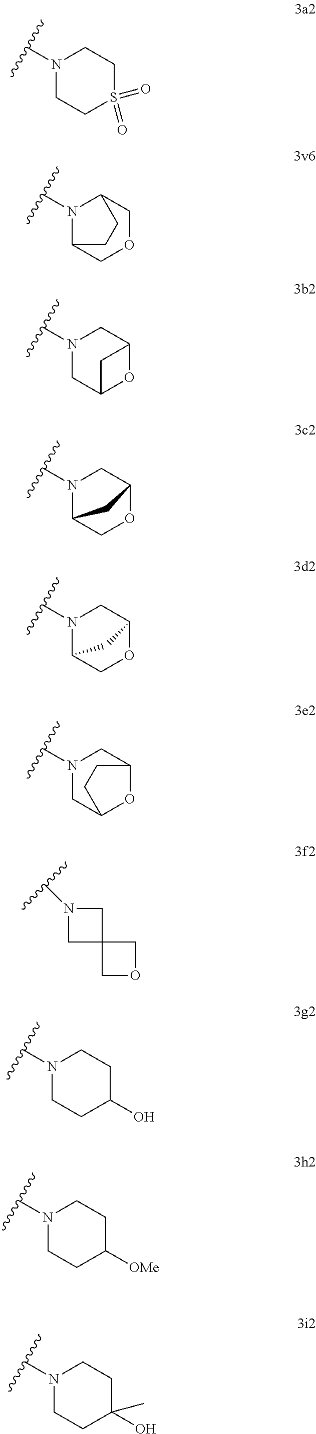

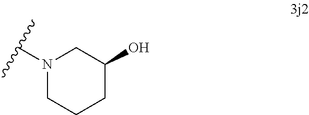 [SMILES: *N1CCC[C@H](O)C1]

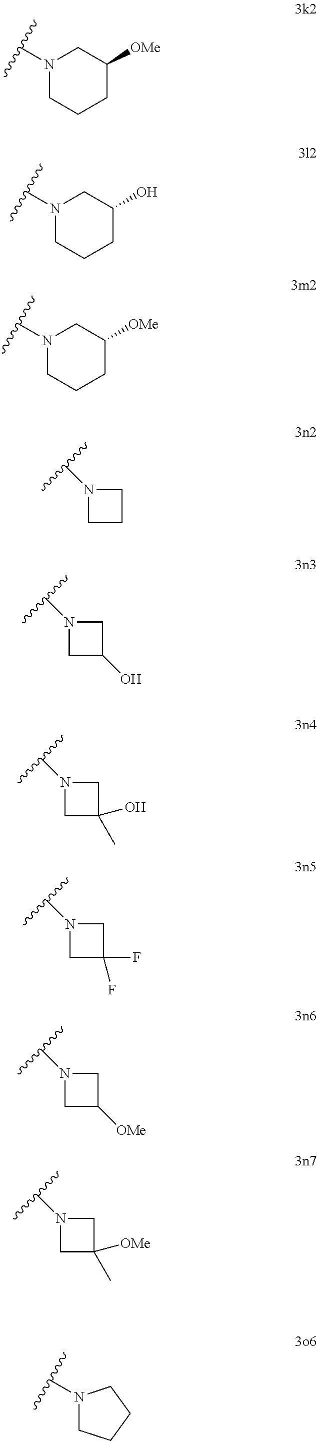

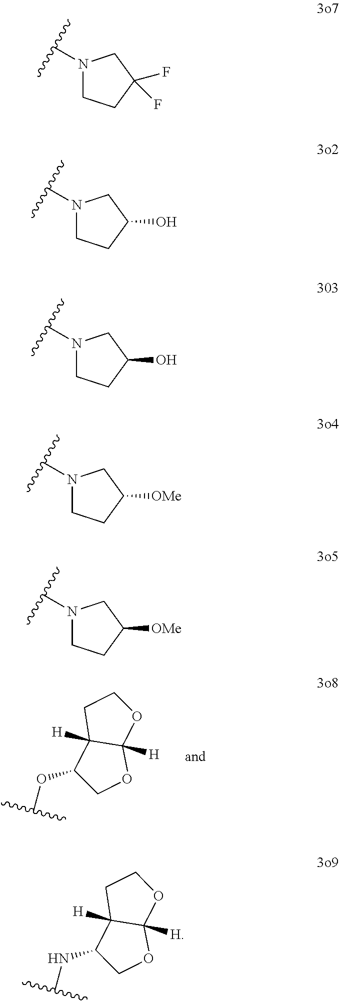

In some embodiments, R₄ is alkyl or a group selected from

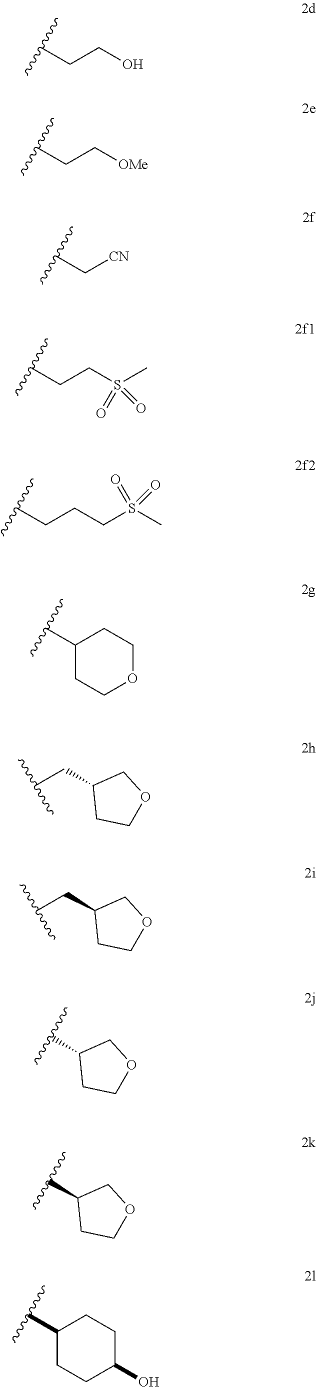

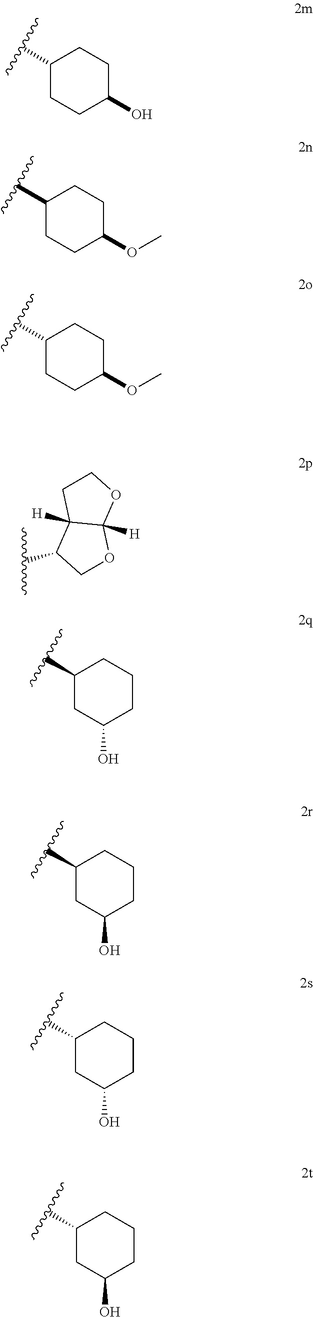

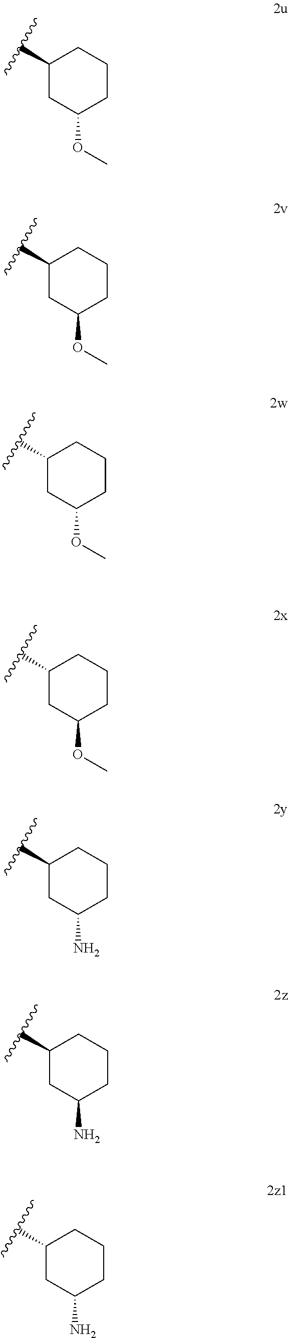

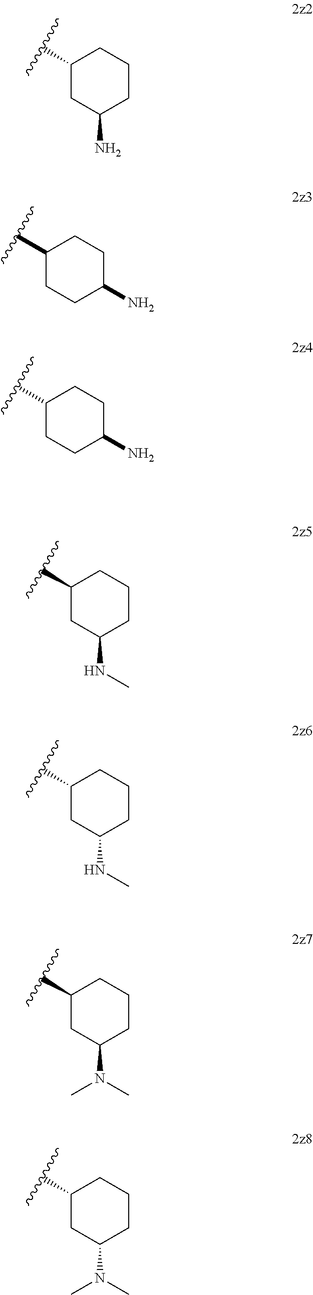

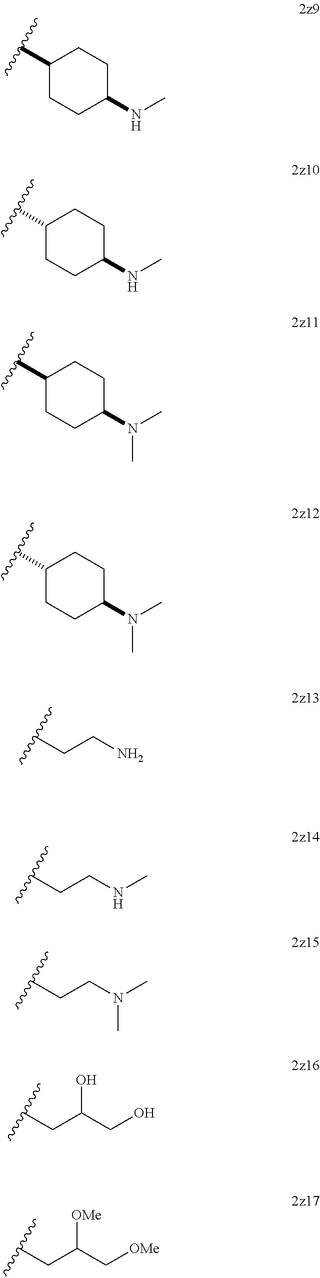

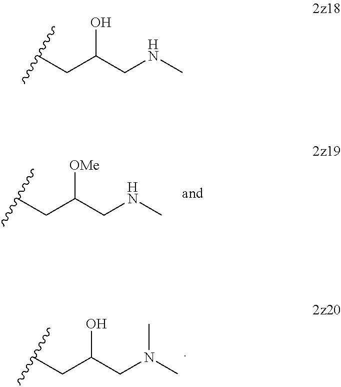

In some embodiments, the compound of formula (I) is 5-((2-aminopyrimidin-5-yl)ethynyl)-N-(4-((4-methylpiperazin-1-yl)methyl)-3-(trifluoromethyl)phenyl)nicotinamide,

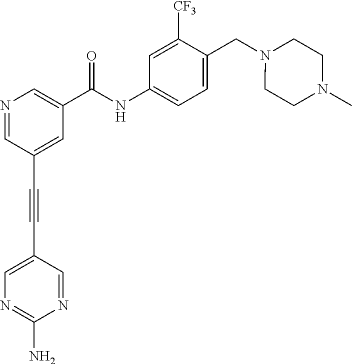 [SMILES: CN1CCN(Cc2ccc(NC(=O)c3cncc(C#Cc4cnc(N)nc4)c3)cc2C(F)(F)F)CC1]

or a pharmaceutically acceptable salt, hydrate, tautomer, or optical isomer thereof.

In some embodiments, the compound of formula (I) is 5-((3-amino-1,2,4-triazin-5-yl)ethynyl)-N-(4-((4-methylpiperazin-1-yl)methyl)-3-(trifluoromethyl)phenyl)nicotinamide,

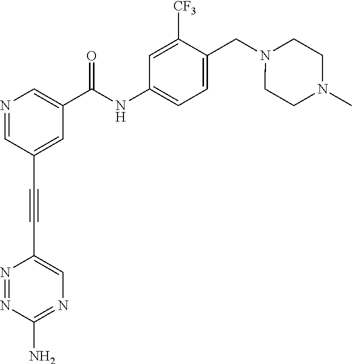 [SMILES: CN1CCN(Cc2ccc(NC(=O)c3cncc(C#Cc4cnc(N)nn4)c3)cc2C(F)(F)F)CC1]

or a pharmaceutically acceptable salt, hydrate, tautomer, or optical isomer thereof.

In some embodiments, the compound of formula (I) is,

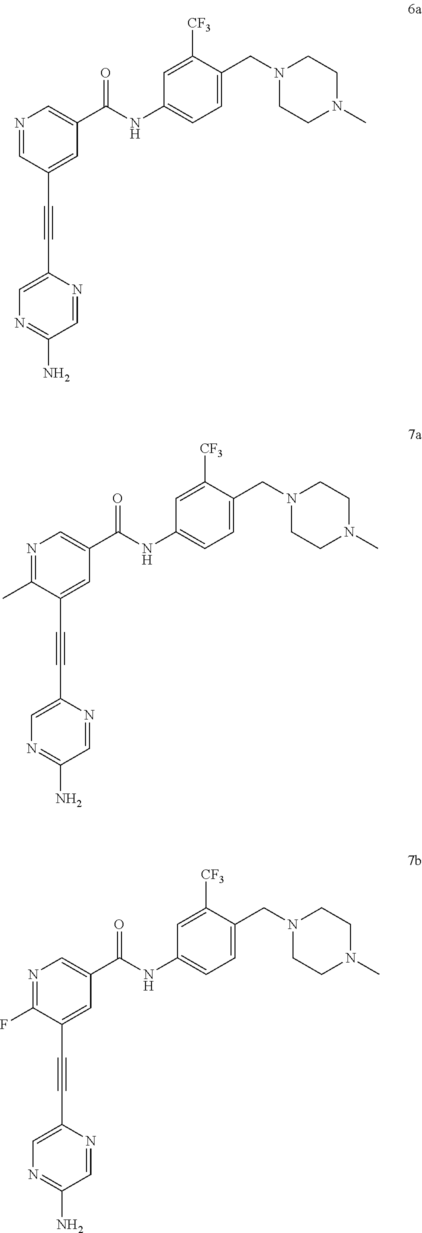

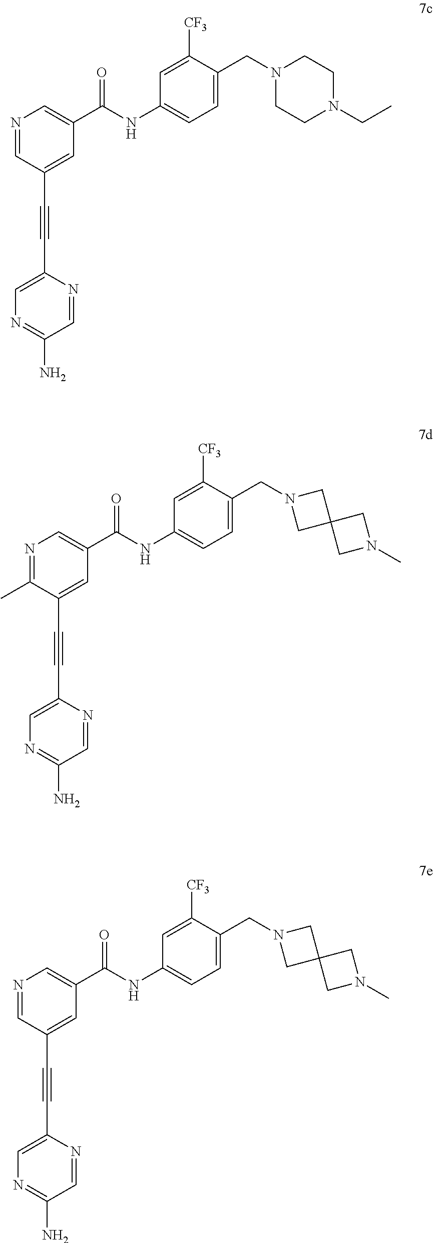

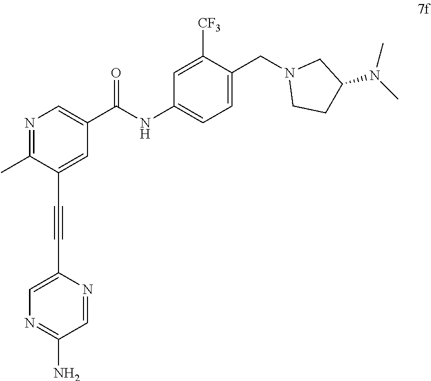

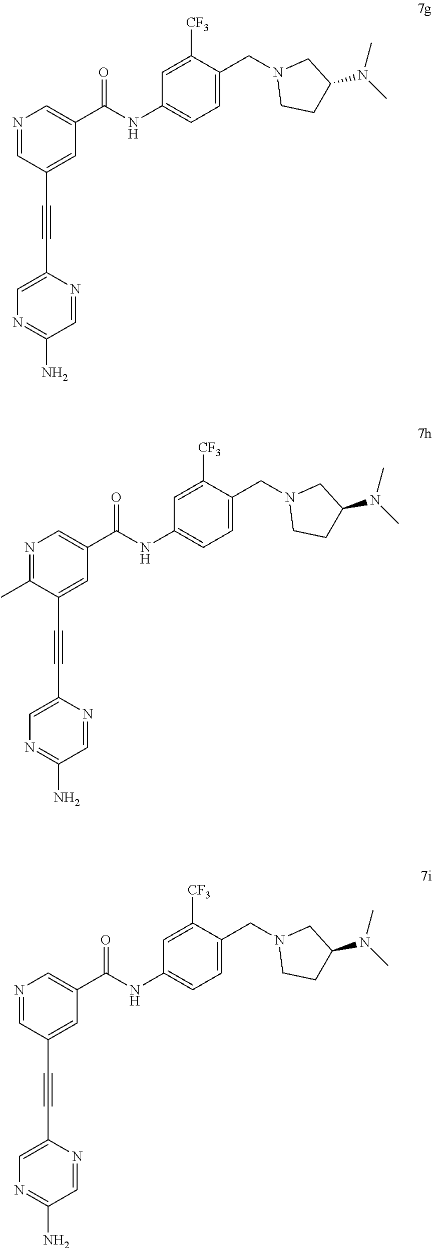

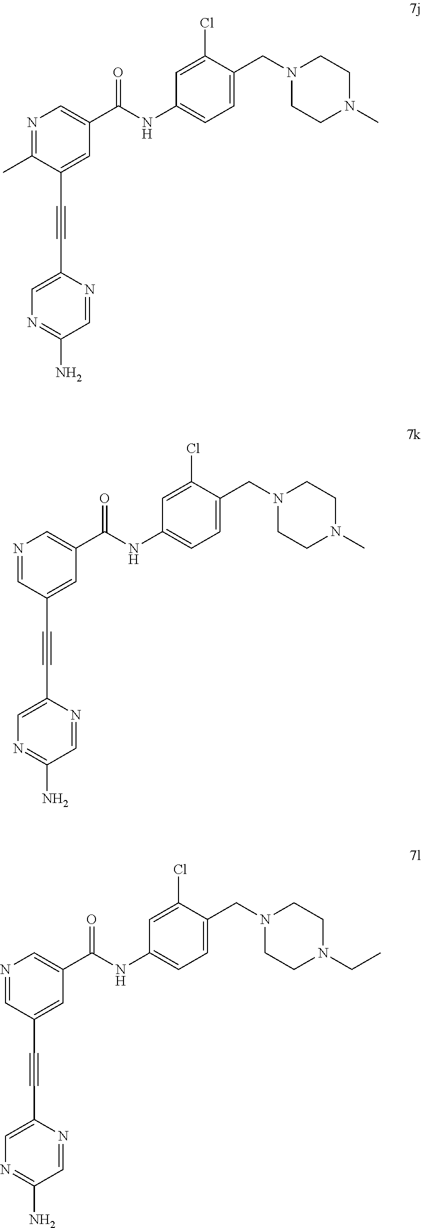

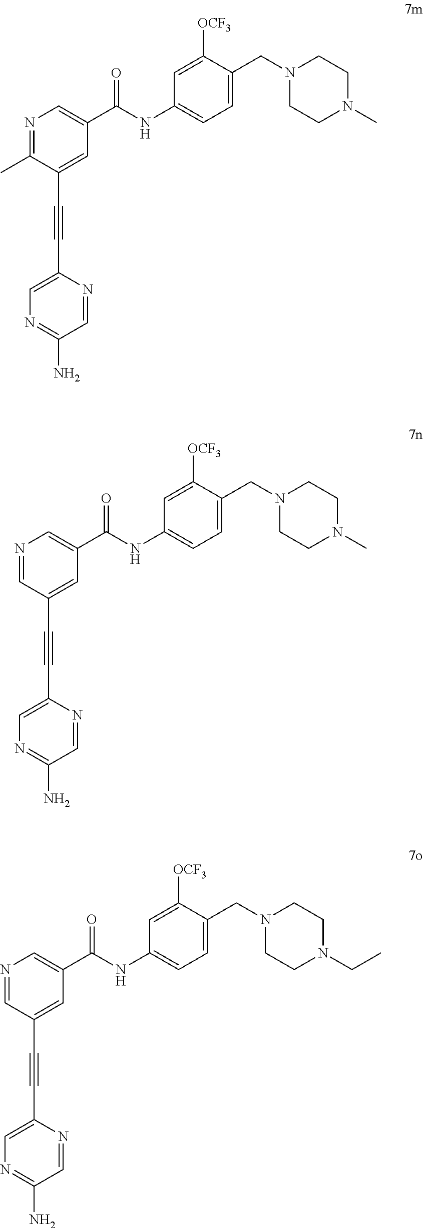

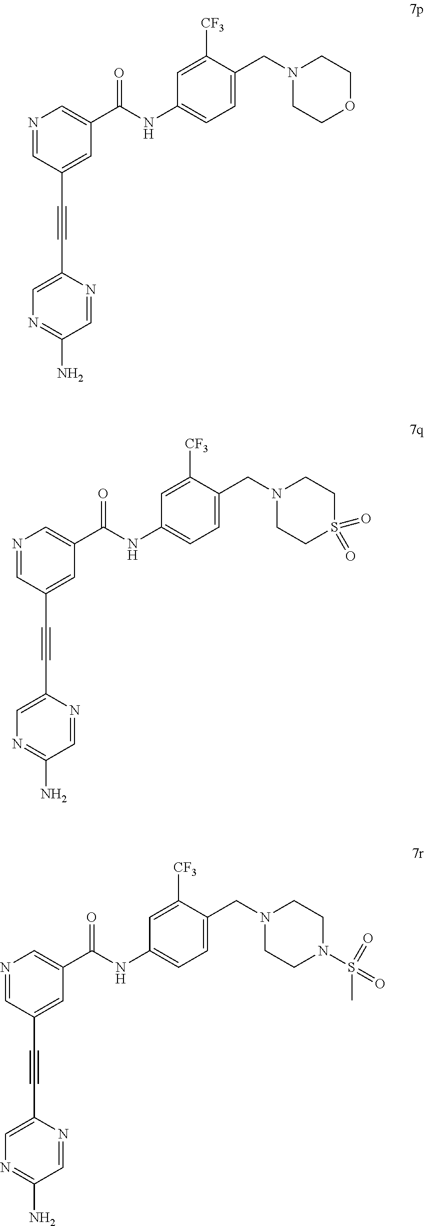 [SMILES: Nc1cnc(C#Cc2cncc(C(=O)Nc3ccc(CN4CCOCC4)c(C(F)(F)F)c3)c2)cn1 ; Nc1cnc(C#Cc2cncc(C(=O)Nc3ccc(CN4CCS(=O)(=O)CC4)c(C(F)(F)F)c3)c2)cn1 ; CS(=O)(=O)N1CCN(Cc2ccc(NC(=O)c3cncc(C#Cc4cnc(N)cn4)c3)cc2C(F)(F)F)CC1]

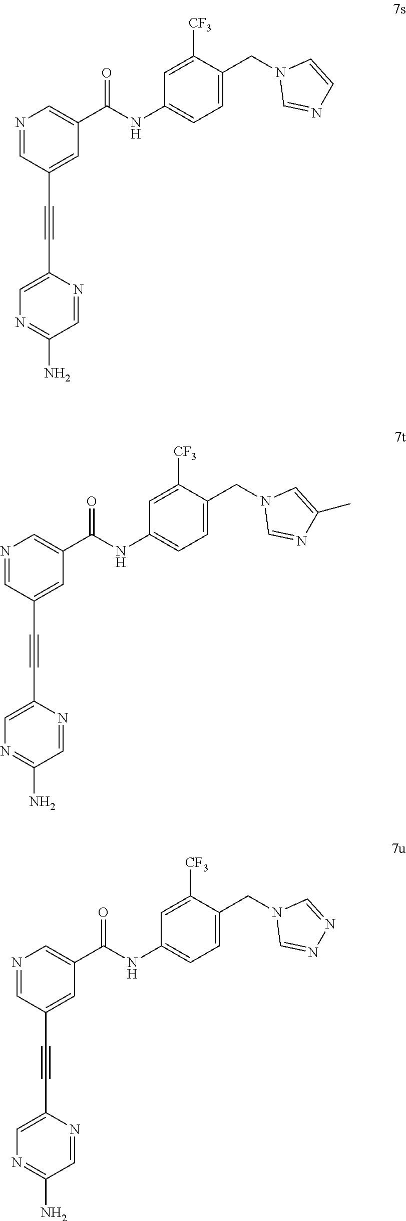 [SMILES: Nc1cnc(C#Cc2cncc(C(=O)Nc3ccc(Cn4ccnc4)c(C(F)(F)F)c3)c2)cn1 ; Cc1cn(Cc2ccc(NC(=O)c3cncc(C#Cc4cnc(N)cn4)c3)cc2C(F)(F)F)cn1 ; Nc1cnc(C#Cc2cncc(C(=O)Nc3ccc(Cn4cnnc4)c(C(F)(F)F)c3)c2)cn1]

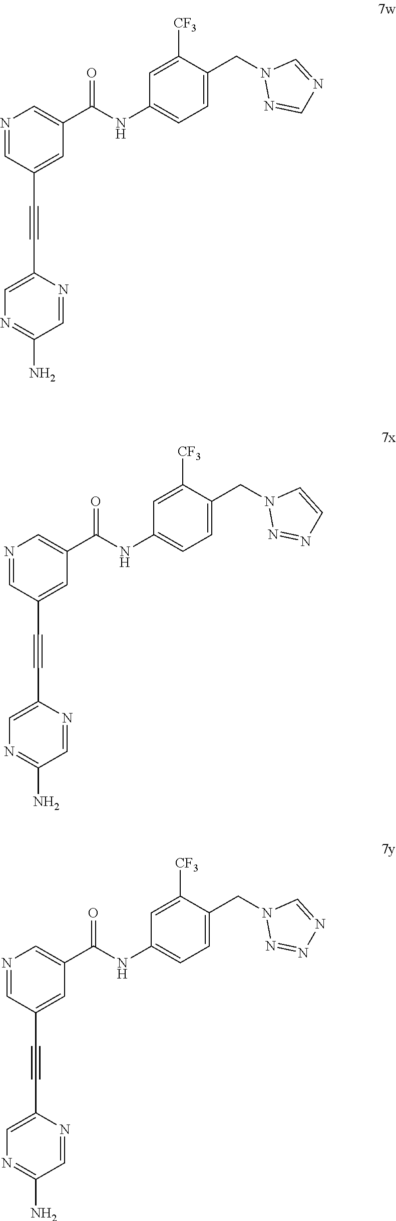 [SMILES: Nc1cnc(C#Cc2cncc(C(=O)Nc3ccc(Cn4cncn4)c(C(F)(F)F)c3)c2)cn1 ; Nc1cnc(C#Cc2cncc(C(=O)Nc3ccc(Cn4ccnn4)c(C(F)(F)F)c3)c2)cn1 ; Nc1cnc(C#Cc2cncc(C(=O)Nc3ccc(Cn4cnnn4)c(C(F)(F)F)c3)c2)cn1]

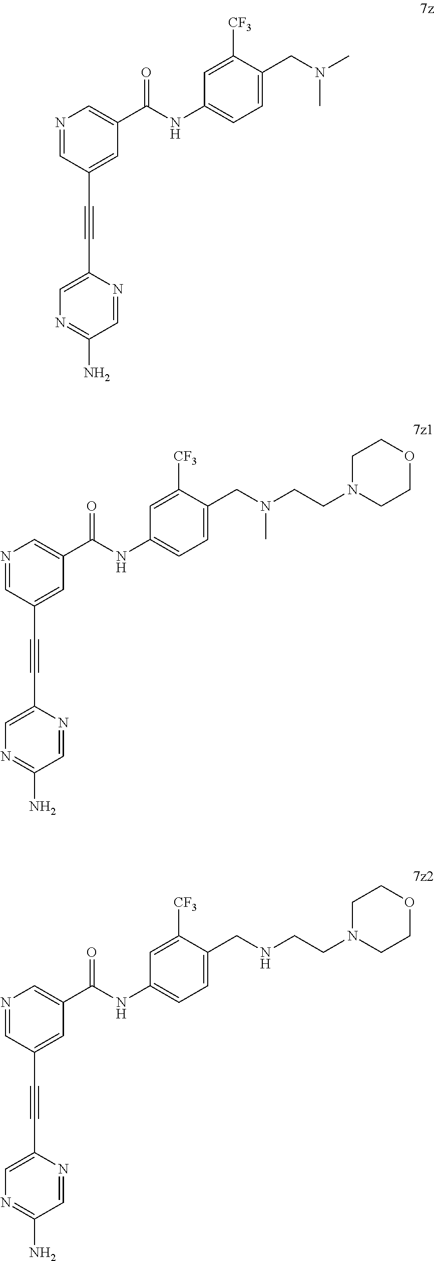 [SMILES: CN(C)Cc1ccc(NC(=O)c2cncc(C#Cc3cnc(N)cn3)c2)cc1C(F)(F)F ; CN(CCN1CCOCC1)Cc1ccc(NC(=O)c2cncc(C#Cc3cnc(N)cn3)c2)cc1C(F)(F)F ; Nc1cnc(C#Cc2cncc(C(=O)Nc3ccc(CNCCN4CCOCC4)c(C(F)(F)F)c3)c2)cn1]

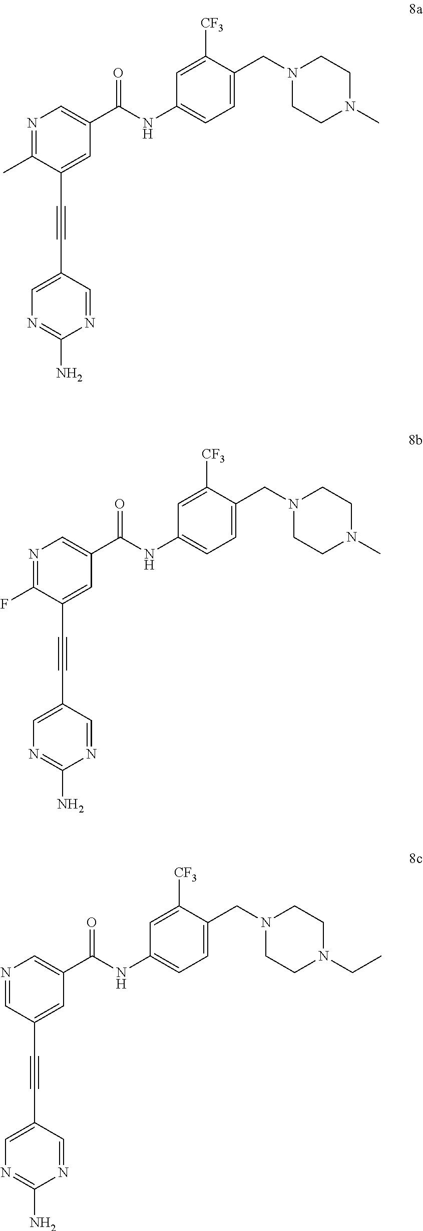

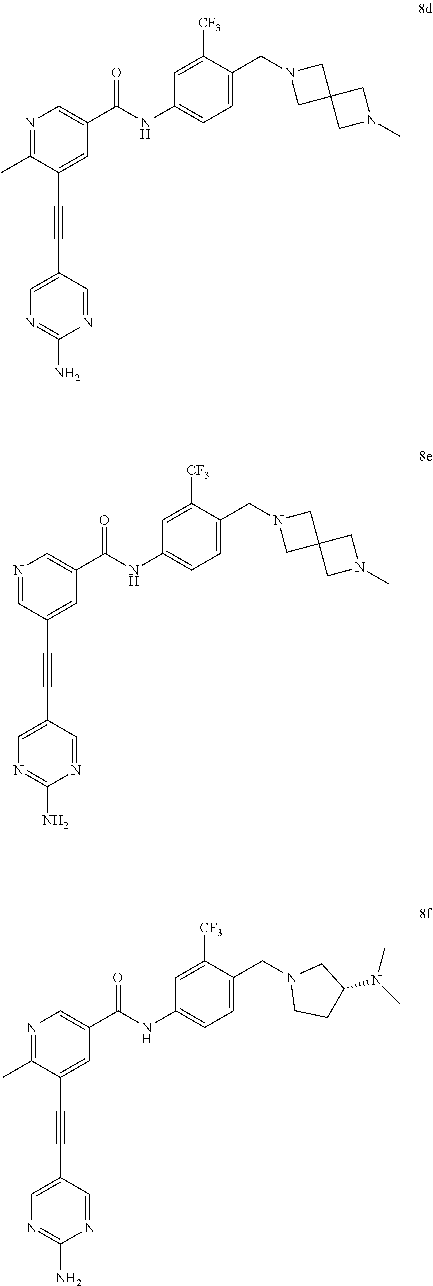

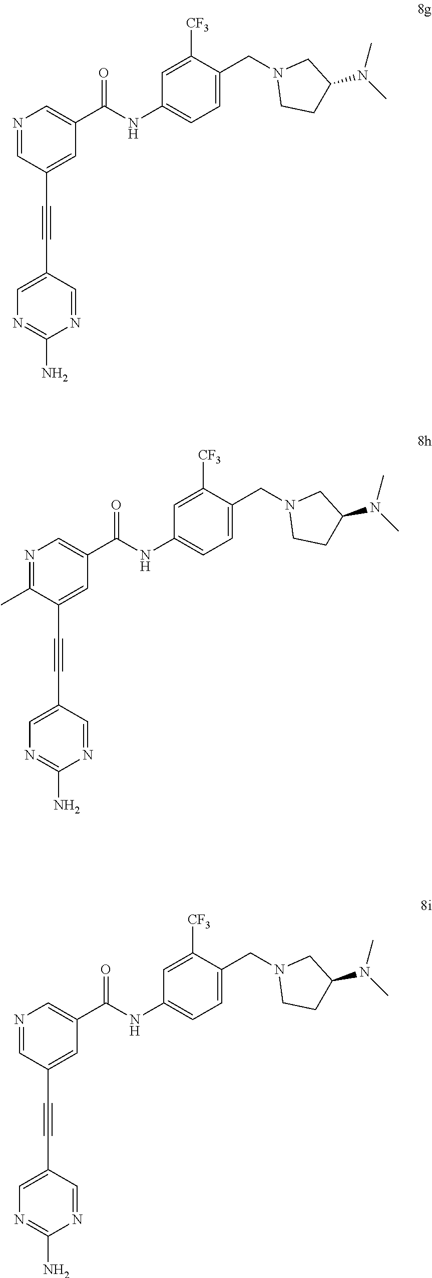

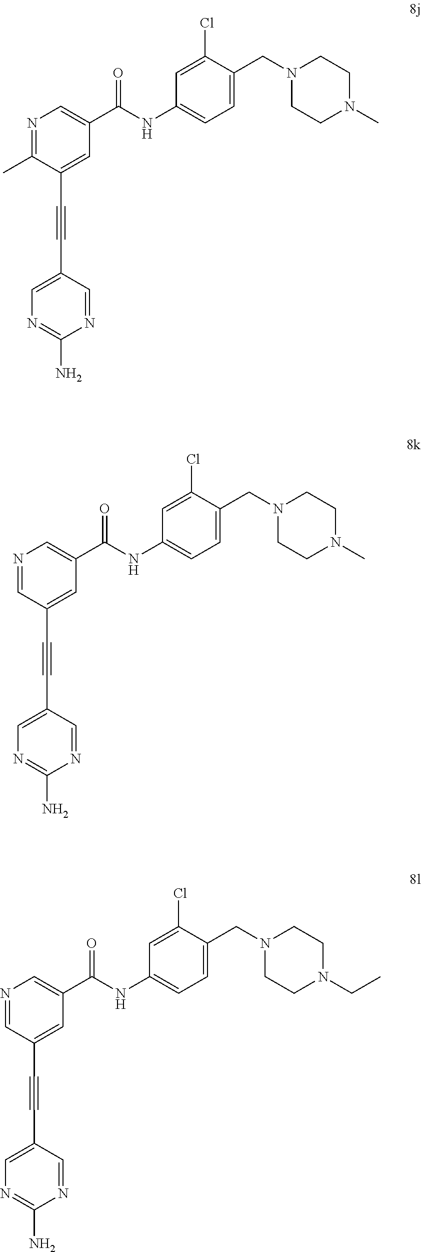

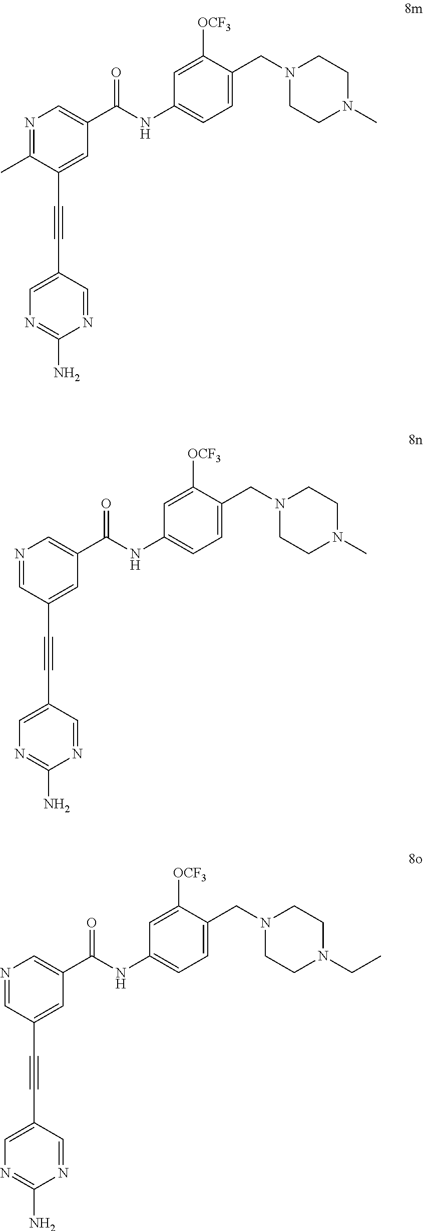

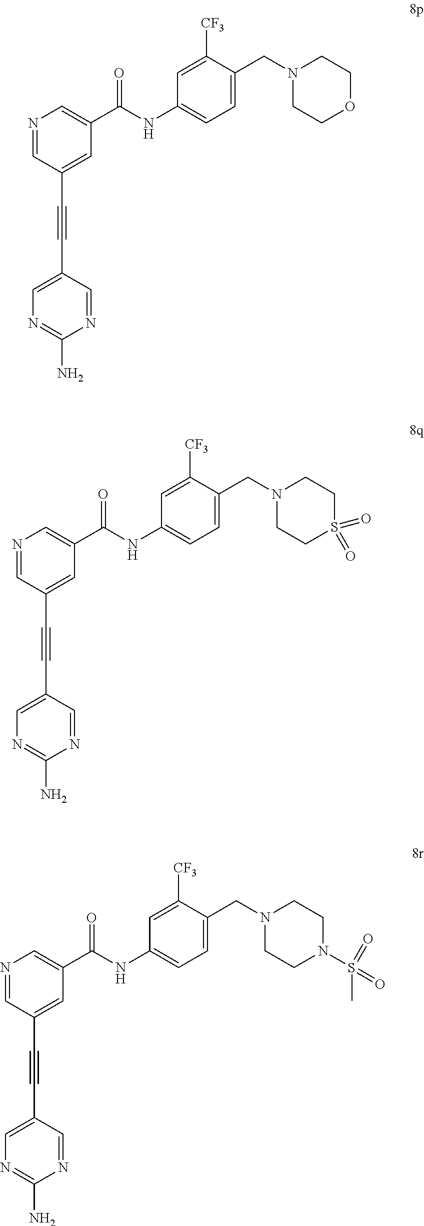 [SMILES: Nc1ncc(C#Cc2cncc(C(=O)Nc3ccc(CN4CCOCC4)c(C(F)(F)F)c3)c2)cn1 ; Nc1ncc(C#Cc2cncc(C(=O)Nc3ccc(CN4CCS(=O)(=O)CC4)c(C(F)(F)F)c3)c2)cn1 ; CS(=O)(=O)N1CCN(Cc2ccc(NC(=O)c3cncc(C#Cc4cnc(N)nc4)c3)cc2C(F)(F)F)CC1]

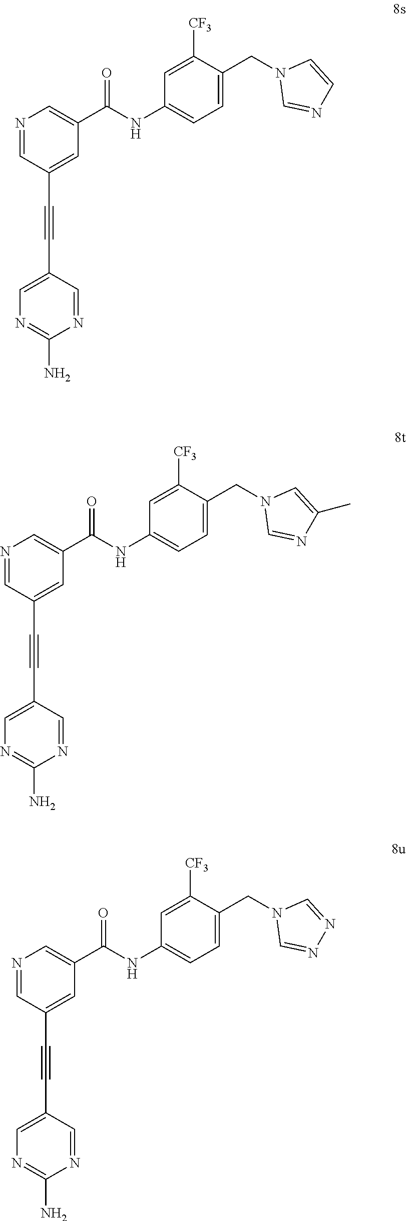 [SMILES: Nc1ncc(C#Cc2cncc(C(=O)Nc3ccc(Cn4ccnc4)c(C(F)(F)F)c3)c2)cn1 ; Cc1cn(Cc2ccc(NC(=O)c3cncc(C#Cc4cnc(N)nc4)c3)cc2C(F)(F)F)cn1 ; Nc1ncc(C#Cc2cncc(C(=O)Nc3ccc(Cn4cnnc4)c(C(F)(F)F)c3)c2)cn1]

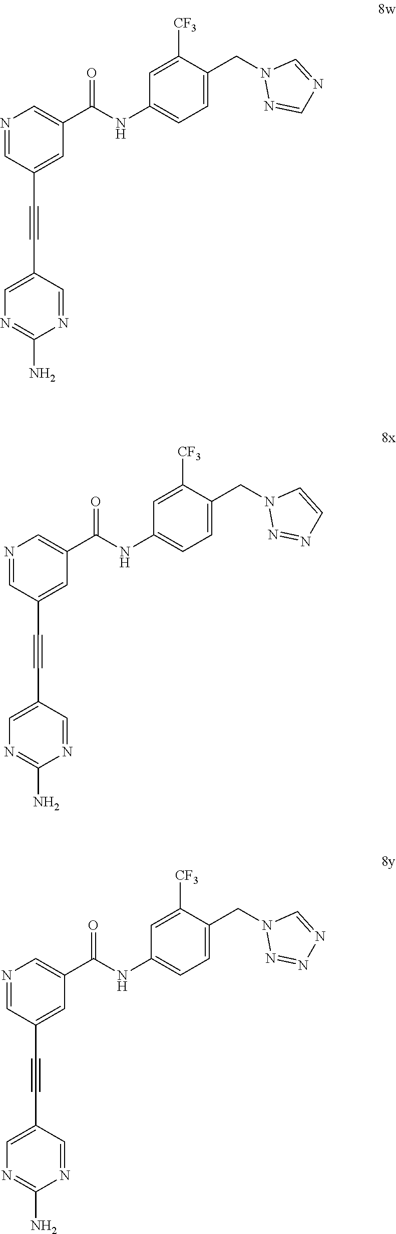 [SMILES: Nc1ncc(C#Cc2cncc(C(=O)Nc3ccc(Cn4cncn4)c(C(F)(F)F)c3)c2)cn1 ; Nc1ncc(C#Cc2cncc(C(=O)Nc3ccc(Cn4ccnn4)c(C(F)(F)F)c3)c2)cn1 ; Nc1ncc(C#Cc2cncc(C(=O)Nc3ccc(Cn4cnnn4)c(C(F)(F)F)c3)c2)cn1]

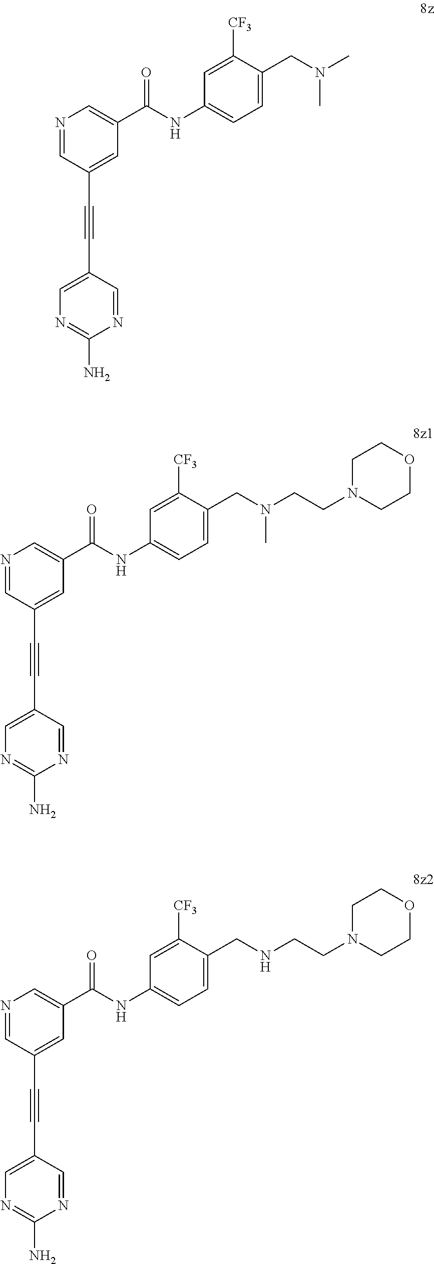 [SMILES: CN(C)Cc1ccc(NC(=O)c2cncc(C#Cc3cnc(N)nc3)c2)cc1C(F)(F)F ; CN(CCN1CCOCC1)Cc1ccc(NC(=O)c2cncc(C#Cc3cnc(N)nc3)c2)cc1C(F)(F)F ; Nc1ncc(C#Cc2cncc(C(=O)Nc3ccc(CNCCN4CCOCC4)c(C(F)(F)F)c3)c2)cn1]

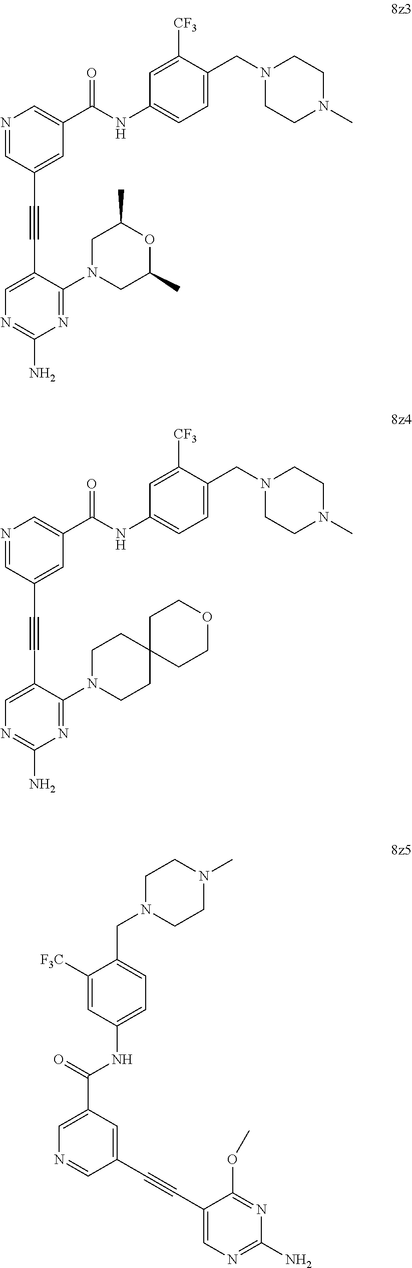 [SMILES: C[C@@H]1CN(c2nc(N)ncc2C#Cc2cncc(C(=O)Nc3ccc(CN4CCN(C)CC4)c(C(F)(F)F)c3)c2)C[C@H](C)O1 ; CN1CCN(Cc2ccc(NC(=O)c3cncc(C#Cc4cnc(N)nc4N4CCC5(CCOCC5)CC4)c3)cc2C(F)(F)F)CC1 ; COc1nc(N)ncc1C#Cc1cncc(C(=O)Nc2ccc(CN3CCN(C)CC3)c(C(F)(F)F)c2)c1]

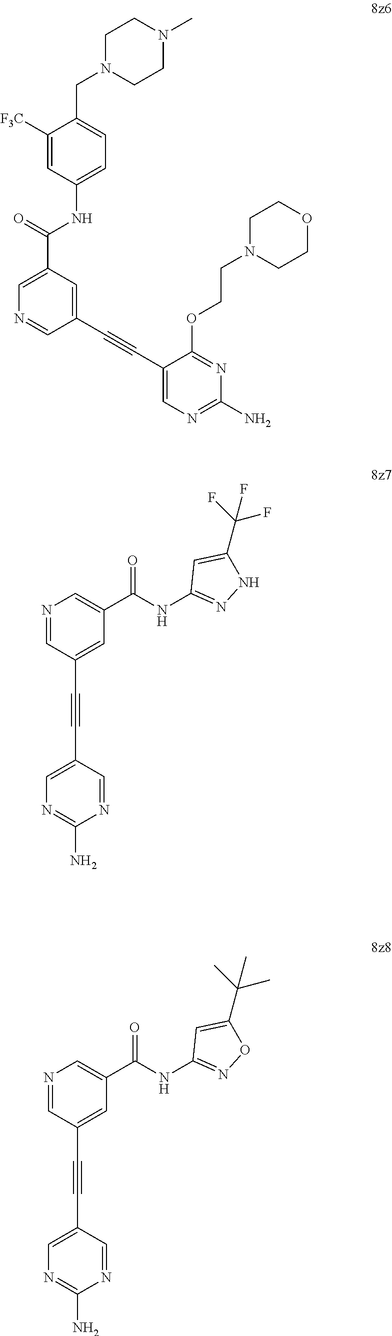 [SMILES: CN1CCN(Cc2ccc(NC(=O)c3cncc(C#Cc4cnc(N)nc4OCCN4CCOCC4)c3)cc2C(F)(F)F)CC1 ; Nc1ncc(C#Cc2cncc(C(=O)Nc3cc(C(F)(F)F)[nH]n3)c2)cn1 ; CC(C)(C)c1cc(NC(=O)c2cncc(C#Cc3cnc(N)nc3)c2)no1]

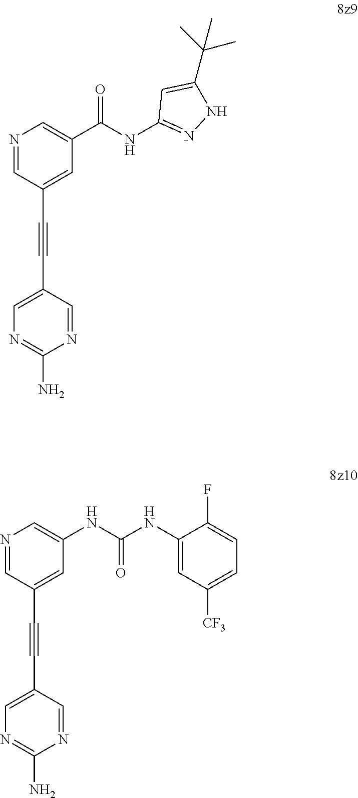 [SMILES: CC(C)(C)c1cc(NC(=O)c2cncc(C#Cc3cnc(N)nc3)c2)n[nH]1 ; Nc1ncc(C#Cc2cncc(NC(=O)Nc3cc(C(F)(F)F)ccc3F)c2)cn1]

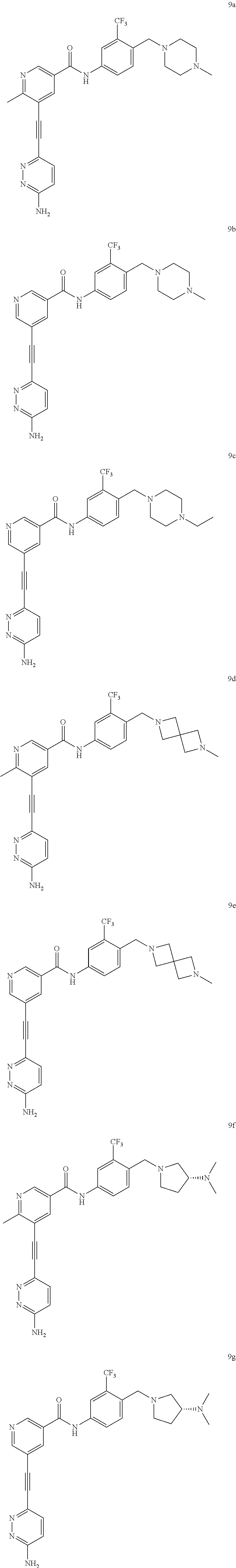

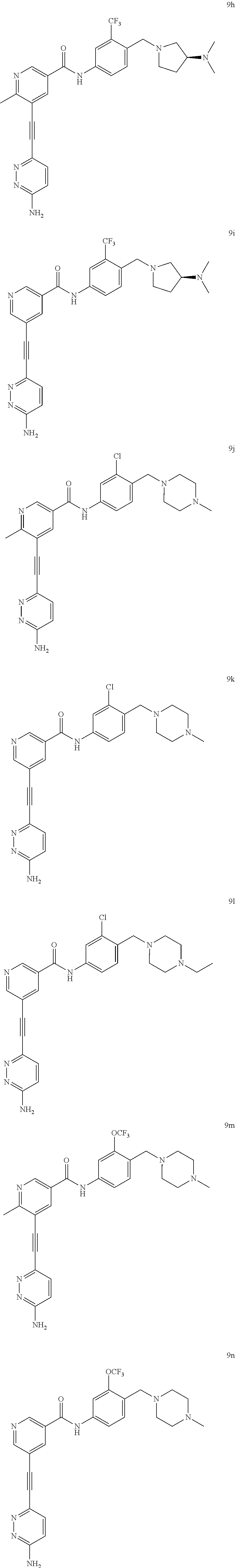

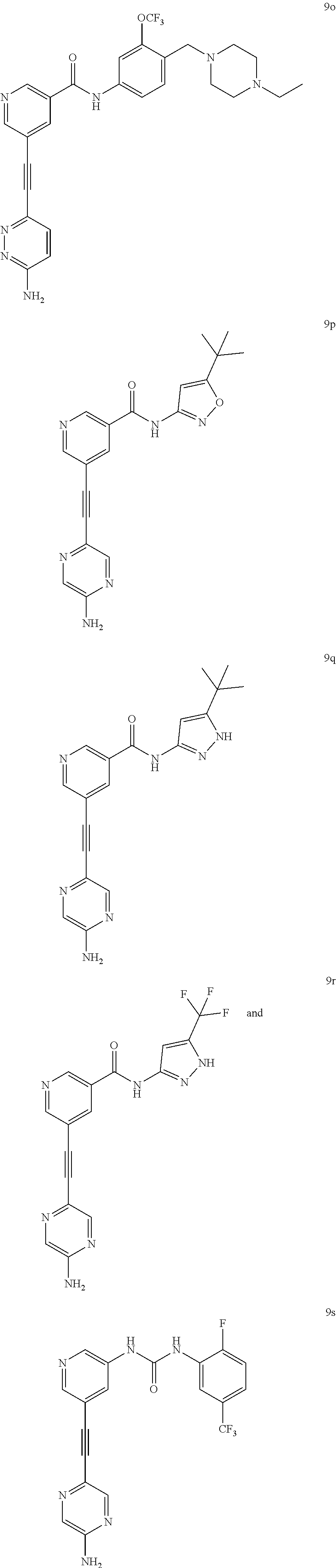 [SMILES: CCN1CCN(Cc2ccc(NC(=O)c3cncc(C#Cc4ccc(N)nn4)c3)cc2OC(F)(F)F)CC1 ; CC(C)(C)c1cc(NC(=O)c2cncc(C#Cc3cnc(N)cn3)c2)no1 ; CC(C)(C)c1cc(NC(=O)c2cncc(C#Cc3cnc(N)cn3)c2)n[nH]1 ; Nc1cnc(C#Cc2cncc(C(=O)Nc3cc(C(F)(F)F)[nH]n3)c2)cn1 ; Nc1cnc(C#Cc2cncc(NC(=O)Nc3cc(C(F)(F)F)ccc3F)c2)cn1]

or a pharmaceutically acceptable salt, hydrate, tautomer, or optical isomer thereof.

In some embodiments, provided is a compound of formula (IA)

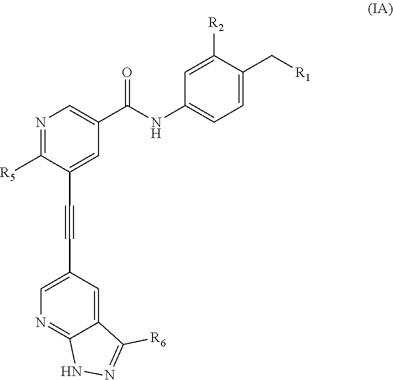

wherein
R₁ is NRₐR_{b}, wherein Rₐ and R_{b} each independently represent a group selected from H, alkyl, cycloalkyl, heterocycloalkyl, aryl, heteroaryl, —(CH₂)ₚ-alkyl. —(CH₂)ₚ-cycloalkyl. —(CH₂)ₚ-heterocycloalkyl, —(CH₂)ₚ—SO₂Re, —(CH₂)ₚ—OR_{c}, and —(CH₂)ₚ—NRₐR_{b}, wherein Rₐ and R_{b} each independently is optionally substituted with at least one of alkyl, halo, OR_{c}, and NH₂; or
Rₐ and R_{b}, together with the nitrogen atom to which they are attached, form a 4- to 8-membered heterocycloalkyl group, which is a mono or a bicyclic ring, either of which is optionally substituted with a group selected from alkyl, cycloalkyl, halo, nitro, CN, SO₂Re, OR_{d}, —(CH₂)ₚ—OR_{c} and NR_{c}R_{c};
R_{c} is H or alkyl;
R_{d} is H, halo, OH, CN, alkyl, —(CH₂)ₚ-alkyl, —(CH₂)ₚ-cycloalkyl, —(CH₂)ₚ-heterocycloalkyl, —(CH₂)ₚ—SO₂R_{d}, —(CH₂)ₚ—OR_{d}, —(CH₂)ₚ—(OR_{d})₂, or —(CH₂)ₚ—NRₐR_{b};
p is 0-5;
R₂ represents a group independently selected from alkyl, CF₃, OCF₃, and halo;
R₅ represents a group independently selected from H, alkyl, and halo;
R₆ represents a group independently selected from H, alkyl, ORₐ, and NRₐR_{b}, wherein Rₐ and R_{b} each independently represent a group selected from H, alkyl, cycloalkyl, heterocycloalkyl, aryl, heteroaryl, —(CH₂)ₚ-alkyl, —(CH₂)ₚ-cycloalkyl, —(CH₂)ₚ-heterocycloalkyl, —(CH₂)ₚ—SO₂R_{c}, —(CH₂)ₚ—OR_{c}, and —(CH₂)ₚ—NRₐR_{b}, wherein Rₐ and R_{b} each independently is optionally substituted with at least one of alkyl, halo, OR₀, and NH₂; or
Rₐ and R_{b}, together with the nitrogen atom to which they are attached, form a 4- to 8-membered heterocycloalkyl group, which is a mono or a bicyclic ring, either of which is optionally substituted with a group selected from alkyl, cycloalkyl, halo, nitro, CN, SO₂R_{c}, OR_{d}, —(CH₂)ₚ—OR, and NR_{c}R_{c}, wherein R_{c}, R_{d} and p are as defined above.

In some embodiments, R₆ is selected from

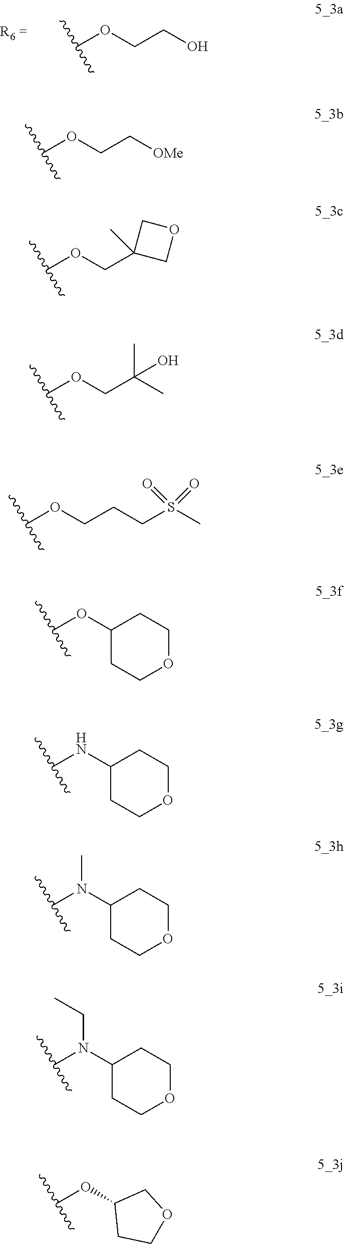

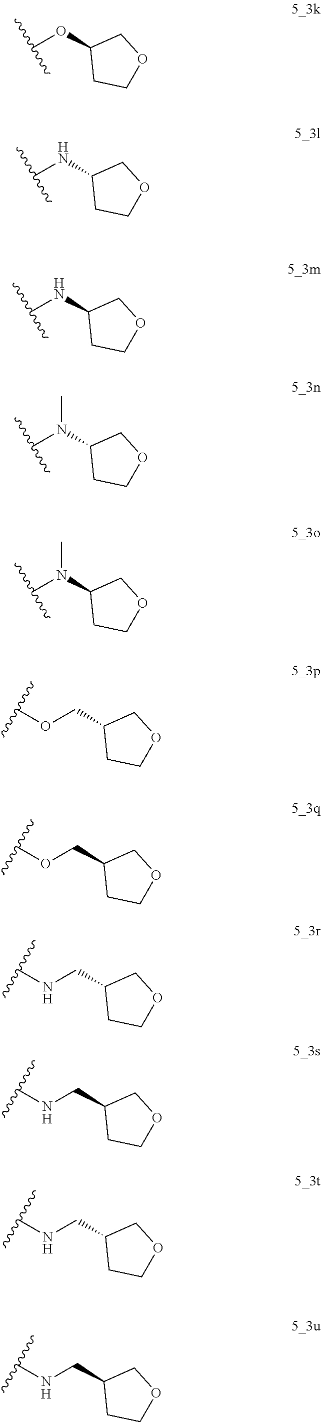

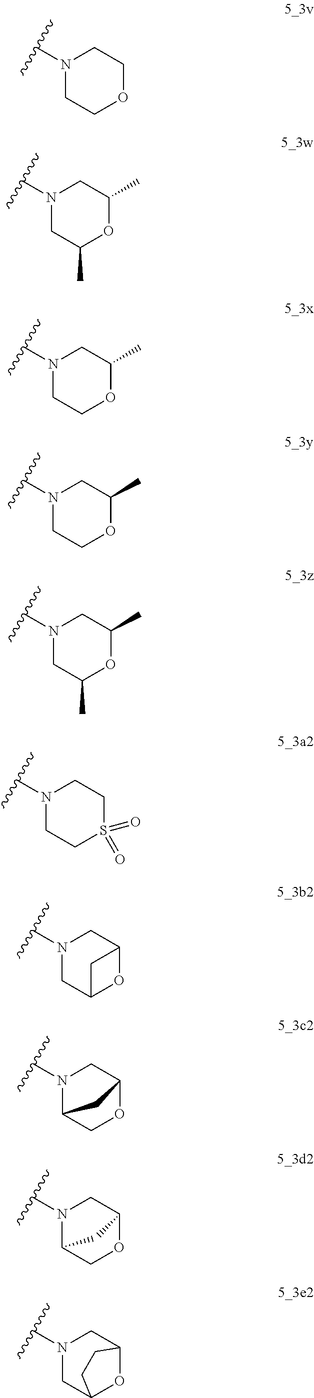

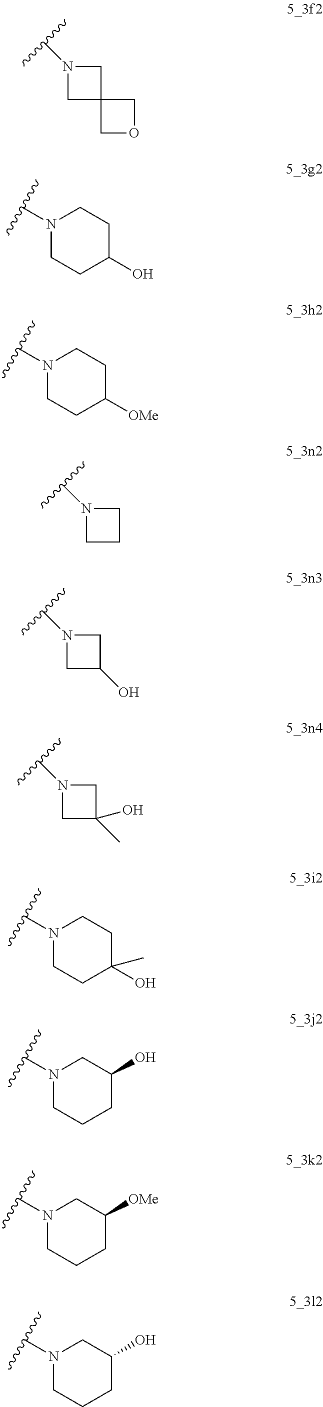

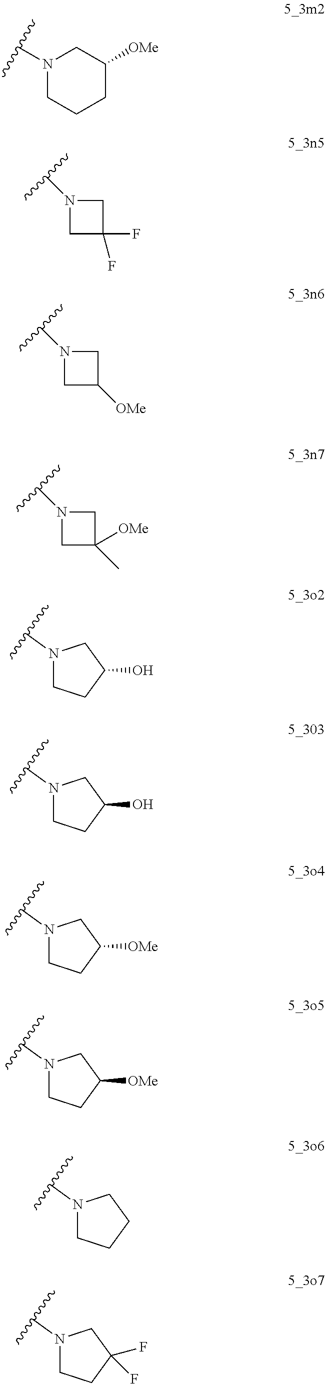

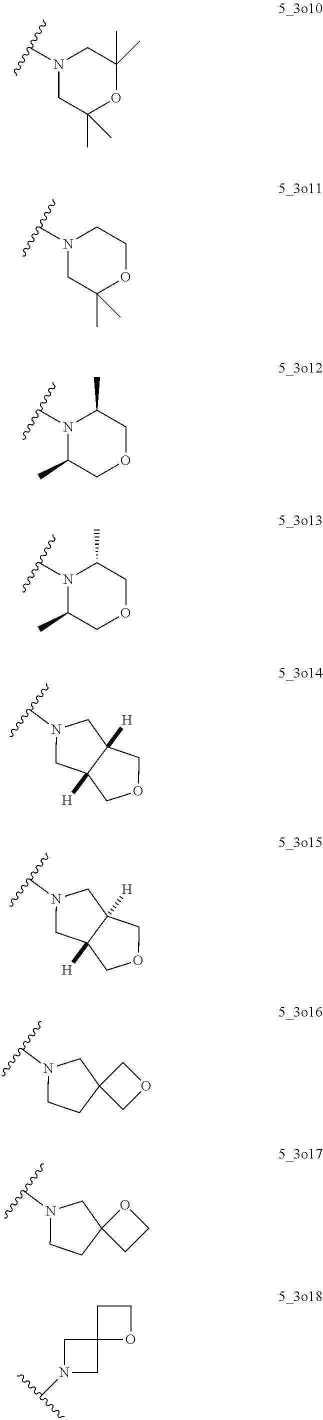

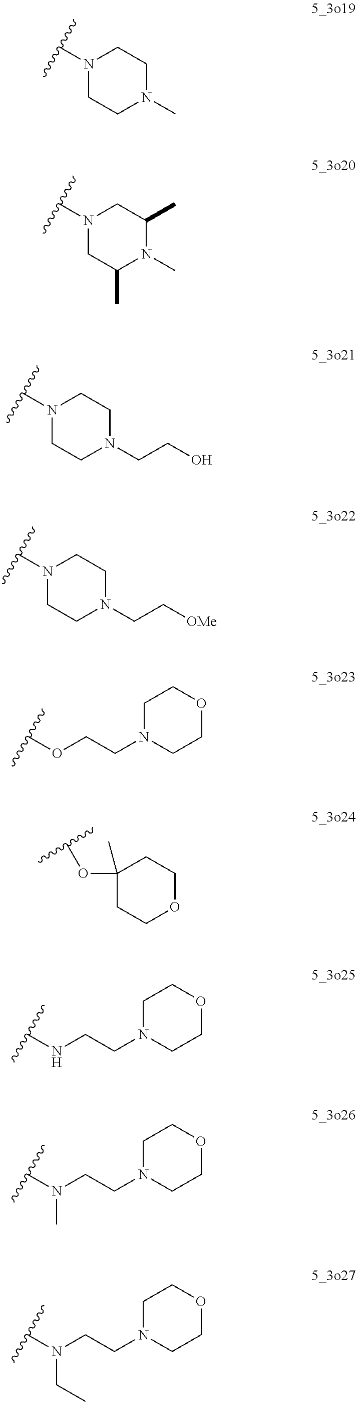

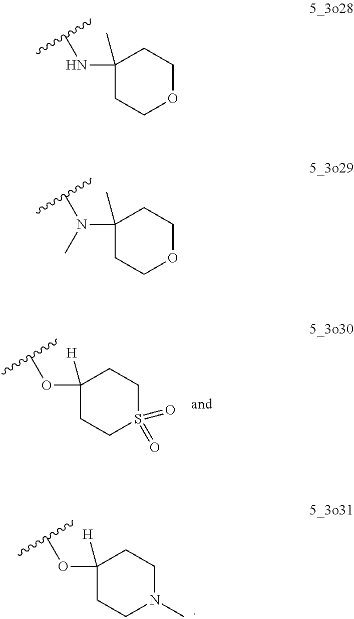

In some embodiments, the compound of formula (IA) is 5-((1H-pyrazolo[3,4-b]pyridin-5-yl)ethynyl)-N-(4-((4-methylpiperazin-1-yl)methyl)-3-(trifluoromethyl)phenyl)nicotinamide,

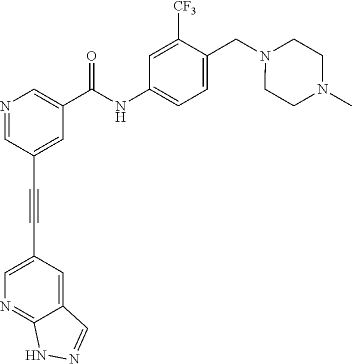 [SMILES: CN1CCN(Cc2ccc(NC(=O)c3cncc(C#Cc4cnc5[nH]ncc5c4)c3)cc2C(F)(F)F)CC1]

or a pharmaceutically acceptable salt, hydrate, tautomer, or optical isomer thereof.

In some embodiments, the compound of formula (IA) is 5-((3-methyl-1H-pyrazolo[3,4-b]pyridin-5-yl)ethynyl)-N-(4-((4-methylpiperazin-1-yl)methyl)-3-(trifluoromethyl)phenyl)nicotinamide,

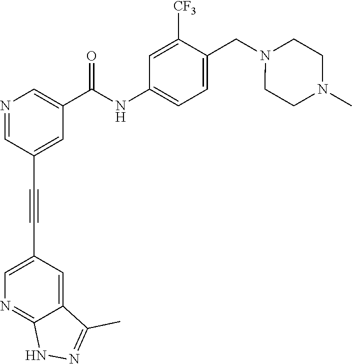 [SMILES: Cc1n[nH]c2ncc(C#Cc3cncc(C(=O)Nc4ccc(CN5CCN(C)CC5)c(C(F)(F)F)c4)c3)cc12]

or a pharmaceutically acceptable salt, hydrate, tautomer, or optical isomer thereof.

In some embodiments, the compound of formula (IA) is,

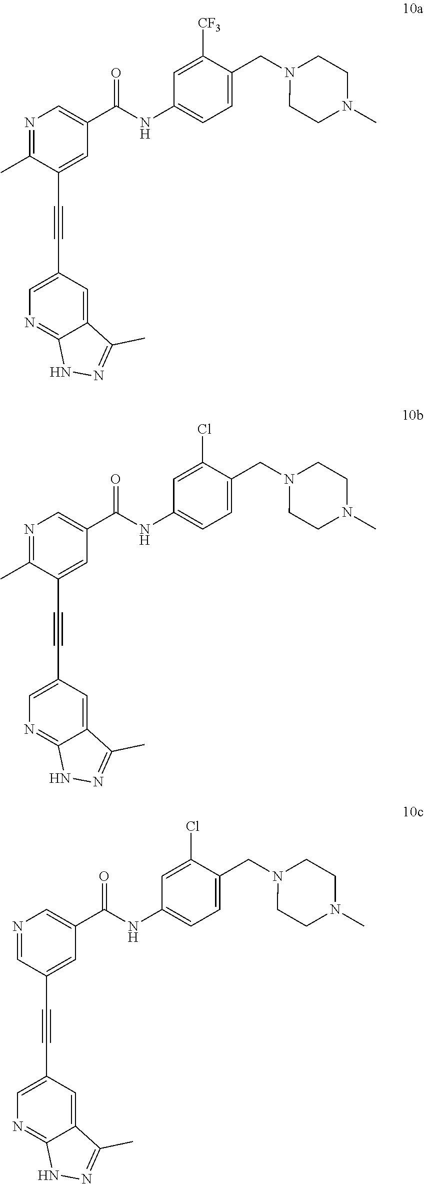

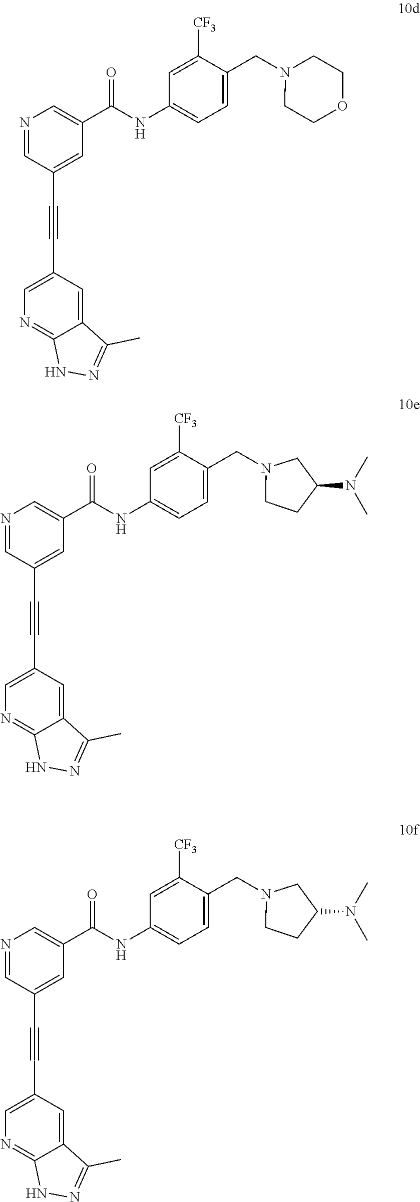

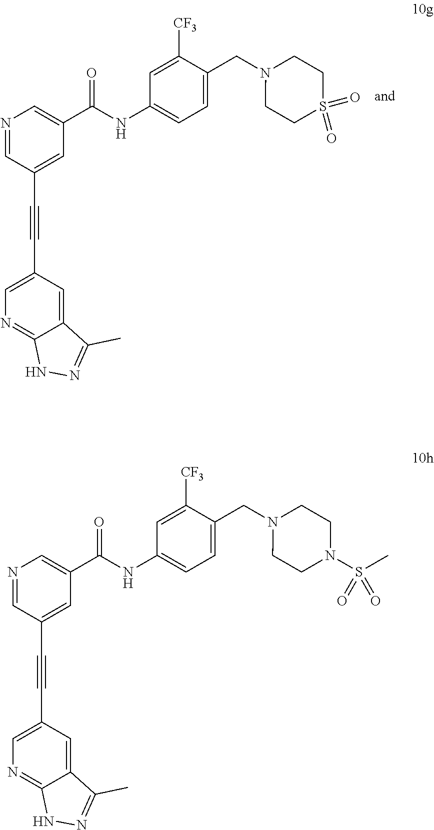 [SMILES: Cc1n[nH]c2ncc(C#Cc3cncc(C(=O)Nc4ccc(CN5CCS(=O)(=O)CC5)c(C(F)(F)F)c4)c3)cc12 ; Cc1n[nH]c2ncc(C#Cc3cncc(C(=O)Nc4ccc(CN5CCN(S(C)(=O)=O)CC5)c(C(F)(F)F)c4)c3)cc12]

or a pharmaceutically acceptable salt, hydrate, tautomer, or optical isomer thereof.

The term “substituted” refers to a functional group in which one or more hydrogen atoms contained therein are replaced by one or more non-hydrogen atoms. The term “functional group” or “substituent” refers to a group that can be or is substituted onto a molecule. Examples of substituents or functional groups include, but are not limited to, a halo (e.g., F, Cl, Br, and I); an oxygen atom in groups such as hydroxyl groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, azides, hydroxylamines, cyano, nitro groups, N-oxides, hydrazides, and enamines; and other heteroatoms in various other groups.

Non-limiting examples of substituents, which can be bonded to a substituted carbon atom (or other atom, such as nitrogen) include F, Cl, Br, I, OR. OC(O)N(R)₂, CN, NO. NO₂. ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, (CH₂)₀₋₂P(O)OR₂, C(O)R, C(O)C(O)R, C(O)CH₂C(O)R, C(S)R, C(O)OR, OC(O)R, C(O)N(R)₂, OC(O)N(R)₂. C(S)N(R)₂, (CH₂)₀₋₂N(R)C(O)R, (CH₂)₀₋₂N(R)C(O)OR, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R wherein R can be hydrogen or a carbon-based moiety, and wherein the carbon-based moiety can itself be further substituted; for example, where R can be hydrogen, alkyl, acyl, cycloalkyl, aryl, aralkyl, heterocyclyl, heteroaryl, or heteroarylalkyl, any alkyl, acyl, cycloalkyl, aryl, aralkyl, heterocyclvl, heteroaryl, or heteroarylalkyl or R can be independently mono- or multi-substituted; or when two R groups bonded to a nitrogen atom or to adjacent nitrogen atoms can, together with the nitrogen atom or atoms to which they are bonded, form a heterocyclyl, the heterocycle can be mono- or independently multi-substituted.

The term “alkyl” refers to substituted or unsubstituted straight-chain and branched alkyl groups and cycloalkyl groups having from 1 to about 20 carbon atoms (C₁-C₂₀), 1 to 12 carbons (C₁-C₁₂), 1 to 8 carbon atoms (C₁-C₈), or, in some embodiments, from 1 to 6 carbon atoms (C₁-C₆). Examples of straight-chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, tert-butyl, neopentyl, isopentyl, and 2,2-dimethylpropyl groups. The term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” refers to substituted or unsubstituted straight-chain and branched divalent alkenyl and cycloalkenyl groups having from 2 to 20 carbon atoms (C₂-C₂₀), 2 to 12 carbons (C₂-C₁₂), 2 to 8 carbon atoms (C₂-C₈) or, in some embodiments, from 2 to 4 carbon atoms (C₂-C₄) and at least one carbon-carbon double bond. Examples of straight-chain alkenyl groups include those with from 2 to 8 carbon atoms such as —CH═CH—, —CH═CHCH₂—, and the like. Examples of branched alkenyl groups include, but are not limited to, —CH═C(CH₃)— and the like.

The term “alkynyl” refers to an unsaturated monovalent chain of carbon atoms, including at least one triple bond, which may be optionally branched. In various embodiments that include alkynyl, illustrative examples include lower alkynyl, such as C₂-C₆, C₂-C₄ alkynyl, and the like.

The term “hydroxyalkyl” refers to alkyl groups substituted with at least one hydroxyl (—OH) group.

The term “cycloalkyl” refers to substituted or unsubstituted cyclic alkyl groups such as, but not limited to, cyclopropyl, cyclobutyl, cyclopentyl, cyclohexyl, cycloheptyl, and cyclooctyl groups. In some embodiments, the cycloalkyl group can have 3 to about 8-12 ring members, whereas in other embodiments, the number of ring carbon atoms ranges from 3 to 4, 5, 6, or 7. In some embodiments, cycloalkyl groups can have 3 to 6 carbon atoms (C₃-C₆). Cycloalkyl groups further include polycyclic cycloalkyl groups such as, but not limited to, norbornyl, adamantyl, bornyl, camphenyl, isocamphenyl, and carenyl groups, and fused rings such as, but not limited to, decalinyl, and the like.

The term “acyl” refers to a group containing a carbonyl moiety wherein the group is bonded via the carbonyl carbon atom. The carbonyl carbon atom is also bonded to another carbon atom, which can be part of a substituted or unsubstituted alkyl, aryl, aralkyl, cycloalkyl, cycloalkylalkyl, heterocyclyl, heterocyclolalkyl, heteroaryl, heteroarylalkyl group or the like. In the special case wherein the carbonyl carbon atom is bonded to a hydrogen, the group is a “fonnyl” group, an acyl group as the tenn is defined herein. An acyl group can include 0 to about 12-40, 6-10, 1-5 or 2-5 additional carbon atoms bonded to the carbonyl group. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and cryloyl groups and the like. When the group containing the carbon atom that is bonded to the carbonyl carbon atom contains a halogen, the group is termed a “haloacyl” group. An example is a trifluoroacetyl group.

The term “aryl” refers to substituted or unsubstituted cyclic aromatic hydrocarbons that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some embodiments, aryl groups contain about 6 to about 14 carbons (C₆-C₁₄) or from 6 to 10 carbon atoms (C₆-C₁₀) in the ring portions of the groups. Aryl groups can be unsubstituted or substituted, as defined herein. Representative substituted aryl groups can be mono-substituted or substituted more than once, such as, but not limited to, 2-, 3-, 4-, 5-, or 6-substituted phenyl or 2-8 substituted naphthyl groups, which can be substituted with carbon or non-carbon groups such as those listed herein.

The term “heteroaryl” represents aromatic ring comprising at least one hetero atom such as N, S, O, or Se. Heteroaryl in the present disclosure may be any hetero aryl. Heteroaryl includes, but is not limited to, pyrrolidinyl, azetidinyl, piperidynyl, piperazinyl, morpholinyl, chromanyl, indolinonyl, isoindolinonyl, furanyl, pyrrolidinyl, pyridinyl, pyrazinyl, pyrimidinyl, triazinyl, thiophenyl, tetrahydrofuranyl, pyrrolyl, oxazolyl, oxadiazolyl, imidazolyl, triazyolyl, tetrazolyl, benzoxazolinyl, benzthiazolinyl, benzimidazolinyl groups, or any combination thereof.

The term “halo” is used to describe chemical compounds which contain one or more halogen atoms, such as fluorine, chlorine, bromine, and iodine.

The term “heterocyclyl” refers to substituted or unsubstituted aromatic and nonaromatic ring compounds containing three or more ring members, of which one or more is a heteroatom such as, but not limited to, B, N, O, and S. Thus, a heterocyclyl can be a cycloheteroalkyl, a heteroaryl or, if polycyclic, any combination thereof. In some embodiments, heterocyclyl groups include 3 to about 20 ring members, whereas other such groups have 3 to about 15 ring members. In some embodiments, heterocyclyl groups can include 3 to 8 carbon atoms (C₃-C₈), 3 to 6 carbon atoms (C₃-C₆) or 6 to 8 carbon atoms (C₆-C₈).

The term “heterocycloalkyl” refers to a non-aromatic heterocycle where one or more of the ring-forming atoms is/are a heteroatom, such as an O, N, or S atom. Heterocycloalkyl groups can include mono- or polycyclic (e.g., having 2, 3 or 4 fused rings) ring systems as well as spirocycles. Example heterocycloalkyl groups include morpholino, thiomorpholino, piperazinyl, tetrahydrofuranyl, tetrahydrothienyl, 2,3-dihydrobenzofuryl, 1,3-benzodioxole, benzo-1,4-dioxane, piperidinyl, pyrrolidinyl, isoxazolidinyl, isothiazolidinyl, pyrazolidinyl, oxazolidinyl, thiazolidinyl, imidazolidinyl, and the like. Also included in the definition of heterocycloalkyl are moieties that have one or more aromatic rings fused (i.e., having a bond in common with) to the nonaromatic heterocyclic ring, for example phthalimidyl, naphthalimidyl, and benzo derivatives of heterocycles. A heterocycloalkyl group having one or more fused aromatic rings can be attached though either the aromatic or non-aromatic portion. Also included in the definition of hetero-cycloalkyl are moieties where one or more ring-forming atoms are substituted by 1 or 2 oxo or sulfido groups. In some embodiments, the heterocycloalkyl group has from 1 to about 20 carbon atoms, and in further embodiments from about 3 to about 20 carbon atoms. In some embodiments, the heterocycloalkyl group contains 3 to about 20, 3 to about 14, 3 to about 7, or 5 to 6 ring-forming atoms. In some embodiments, the heterocycloalkyl group has 1 to about 4, 1 to about 3, or 1 to 2 heteroatoms. In some embodiments, the heterocycloalkyl group contains O to 3 double bonds. In some embodiments, the heterocycloalkyl group contains 0 to 2 triple bonds.

It is understood that each of alkyl, cycloalkyl, alkenyl, cycloalkenyl, alkylene, and heterocycle may be optionally substituted with independently selected groups such as alkyl, haloalkyl, hydroxyalkyl, aminoalkyl, carboxylic acid and derivatives thereof, including esters, amides, and nitrites, hydroxy, alkoxy, acyloxy, amino, alky and dialky-lamino, acylamino, thio, and the like, and combinations thereof.

The terms “optionally substituted” and “optional substituents” indicate that the groups in question are either unsubstituted or substituted with one or more of the substituents specified. When the groups in question are substituted with more than one substituent, the substituents may be the same or different. When used with the terms “independently”, “independently are,” and “independently selected from,” the groups in question may be the same or different. Certain of the herein defined terms may occur more than once in the structure and, upon such occurrence, each term shall be defined independently of the other.

The term “amine” refers to primary, secondary, and tertiary amines having, e.g., the formula N(group)₃ wherein each group can independently be H or non-H, such as alkyl, aryl, cycloalkyl, heterocycloalkyl, aryl, heteroaryl and the like. Amines include, but are not limited to, R—NH₂, for example, alkylamines, arylamines, alkylarylamines; R₂NH, wherein each R is independently selected, such as dialkylamines, diarylamines, aralkylamines, heterocyclylamines and the like; and R₃N, wherein each R is independently selected, such as trialkylamines, dialkylarylamines, alkyldiarylamines, triarylamines, and the like. The term “amine” also includes ammonium ions.

The term “amino group” refers to a substituent of the form —NH₂, —NHR, —NR₂, —NR₃⁺, wherein each R is independently selected, and protonated forms of each, except for —NR₃⁺, which cannot be protonated. Accordingly, any compound substituted with an amino group can be viewed as an amine. An “amino group” can be a primary, secondary, tertiary, or quaternary amino group. An “alkylamino” group includes a monoalkylamino, a dialkylamino, and a trialkylamino group.

The term “compound” as used herein, is meant to include all stereoisomers, geometric isomers, and tautomers of the structures depicted.

The compounds described herein as “optical isomers” may contain one or more chiral centers, or may otherwise be capable of existing as multiple stereoisomers. In various embodiments, the compounds are not limited to any particular stereochemical requirement, and the compounds, and compositions, methods, uses, and medicaments that include them, may be optically pure or any of a variety of stereoisomeric mixtures, including racemic and other mixtures of enantiomers, other mixtures of diastereomers, and the like. Such mixtures of stereoisomers may include a single stereochemical configuration at one or more chiral centers, while including mixtures of stereochemical configuration at one or more other chiral centers.

Similarly, the compounds described herein may include geometric centers, such as cis, trans, E, and Z double bonds. In various embodiments, the compounds are not limited to any particular geometric isomer requirement, and the compounds, and compositions, methods, uses, and medicaments that include them, may be pure or any of a variety of geometric isomer mixtures. Such mixtures of geometric isomers may include a single configuration at one or more double bonds, while including mixtures of geometry at one or more other double bonds.

The compounds of the present disclosure can be prepared by methods well-known in the art, such as Sonogashira Coupling (see, e.g., Bioorg Chem, 2019, 90, 103052, which is hereby specifically incorporated by reference for its teachings regarding same).

Pyrimidin-2-amine and 1H-pyrazolo[3,4-b]pyridine hinge binders, when combined with alkynyl nicotinamide compounds, such as 5-((2-aminopyrimidin-5-yl)ethynyl)-N-(4-((4-methylpiperazin-1-yl)methyl)-3-(trifluoromethyl)phenyl)nicotinamide, and 5-((1H-pyrazolo[3,4-b]pyridin-5-yl)ethynyl)-N-(4-((4-methylpiperazin-1-yl)methyl)-3-(trifluoromethyl)phenyl)nicotinamide, show higher inhibition to RET kinase G810 mutants than other reported alkynyl nicotinamides, such as HSN748. These new analogs also have better drug-like properties, such as solubility, than previously disclosed alkynyl naphthyridines and isoquinolines.

The compound of formula (I) or (IA) or a pharmaceutically acceptable salt hydrate, tautomer, or optical isomer of either of the foregoing selectively inhibits RET kinase mutants. In exemplary embodiments, RET kinase mutants are G810C, G810A, G810R, G810S, and V804M.

In some embodiments, provided is a pharmaceutical composition comprising a compound of formula (I), or a pharmaceutically acceptable salt, hydrate, tautomer, or optical isomer thereof, and a pharmaceutically acceptable carrier, excipient, or diluent.

In some embodiments, provided is a pharmaceutical composition comprising a compound of formula (IA), or a pharmaceutically acceptable salt, hydrate, tautomer, or optical isomer thereof, and a pharmaceutically acceptable carrier, excipient, or diluent. The carrier, excipient, or diluent can vary based on the particular route of administration (see, e.g., Remington's The Science and Practice of Pharmacy. 23^{rd} ed. (2020)). In some embodiments, a pharmaceutical composition further comprises at least one additional pharmaceutically active agent. Pharmaceutical compositions can be prepared by combining one or more compounds of formula (I) or (IA) or a pharmaceutically acceptable salt, hydrate, tautomer, or optical isomer of either of the foregoing with a pharmaceutically acceptable carrier, excipient, or diluent and, optionally, one or more additional pharmaceutically active agents.

Provided is a method of inhibiting a rearranged during transfection (RET) kinase mutants in a patient in need thereof, which method comprises administering to the patient a therapeutically effective amount of the compound of formula (I) or (IA) or a pharmaceutically acceptable salt, hydrate, tautomer or optical isomer of either of the foregoing, optionally as a pharmaceutical composition comprising the same and a pharmaceutically acceptable carrier, excipient, or diluent, whereupon RET kinase mutants in the patient are inhibited. In some embodiments, the patient has RET kinase-altered cancer.

Provided is a method of treating or inhibiting a RET-kinase altered cancer comprising administering to a patient a therapeutically effective amount of a compound of formula (I) or (IA) or a pharmaceutically acceptable salt, hydrate, tautomer, or optical isomer thereof optionally as a pharmaceutical composition comprising the compound and a pharmaceutically acceptable carrier, excipient, or diluent. The method comprises inhibiting RET mutants. In exemplary embodiments, RET kinase mutants include, but are not limited to, G810C, G810A, G810R, G810S, L730V/l, and V804M. In some embodiments, the RET kinase mutant is G810.

Examples of RET kinase-altered cancers include, but are not limited to, lung cancer, thyroid cancer, pancreatic cancer, and breast cancer. In some embodiments, the compound of formula (I) or (IA) or a pharmaceutically acceptable salt, hydrate, tautomer, or optical isomer thereof, can be administered orally.

The terms “treat,” “treating,” “treated,” or “treatment” (with respect to a disease or condition) are an approach for obtaining beneficial or desired results, including and preferably clinical results and includes, but are not limited to, one or more of the following: improving a condition associated with a disease, curing a disease, lessening severity of a disease, delaying progression of a disease, alleviating one or more symptoms associated with a disease, increasing the quality of life of one suffering from a disease, prolonging survival and/or prophylactic or preventative treatment.

The term “pharmaceutical composition” includes a therapeutically effective amount of one or more compounds for treating a cancer patient. The composition may include other components and/or ingredients, including, but not limited to, other therapeutically active compounds and/or one or more pharmaceutically acceptable carriers, diluents, excipients, and the like.

The term “therapeutic effect” refers to a beneficial local or systemic effect in animals, particularly mammals, and more particularly humans, caused by the administration of a compound.

The term “therapeutically effective amount” means the amount of a compound that is effective to treat a disease or a disorder, such as a RET kinase-altered cancer, at a reasonable benefit/risk ratio. The therapeutically effective amount of such compound will vary depending upon the patient and the disease or disorder being treated, the weight and age of the patient, the severity of the disease or disorder, the manner of administration, and the like, which can readily be determined by one of skill in the art.

The compounds can be administered in unit dosage forms and/or compositions containing one or more pharmaceutically acceptable carriers, adjuvants, diluents, excipients, and/or vehicles, and combinations thereof. As used herein, the term “administering” and its formatives generally refer to any and all means of introducing compounds to the patient including, but not limited to, by oral, intravenous, intratumoral, intramuscular, subcutaneous, transdermal, topically, and like routes of administration.

For oral administration, the compounds can be formulated readily by combining the active compound(s) with pharmaceutically acceptable carriers, excipients, or diluents well-known in the art. Such carriers, excipients, or diluents enable the compounds to be formulated as tablets, pills, powders, dragees, capsules, liquids, gels, syrups, slurries, suspensions, solutions, and the like for oral ingestion by a subject to be treated.

Useful dosages of the compounds can be determined by comparing their in vitro activity with their in vivo activity in animal models. Methods of the extrapolation of effective dosages in mice and other animals to human subjects are known in the art. Indeed, the dosage of the compounds can vary significantly depending on the condition of the subject, the age of the subject, the type of disease the subject is experiencing or at risk of experiencing, the particular compounds used, how advanced the pathology is, the route of administration of the compounds and the possibility of co-usage of other therapeutic treatments or additional drugs in combination therapies. The amount of the composition required for use in treatment (e.g., the therapeutically effective amount or dose) will vary not only with the particular application, but also with the salt selected (if applicable) and the characteristics of the subject (such as, for example, age, condition, sex, the subject's body surface area and/or mass, tolerance to drugs) and will ultimately be at the discretion of the attendant physician, clinician, or otherwise.

The compositions comprising the compound (s) can be formulated in a unit dosage form, each dosage containing from about 5 to about 1,000 mg (1 g), more usually about 100 mg to about 500 mg, of the active ingredient.

In some embodiments, the compositions provided herein contain from about 5 mg to about 50 mg of the active ingredient. One having ordinary skill in the art will appreciate that this embodies compounds or compositions containing about 5 mg to about 10 mg, about 10 mg to about 15 mg, about 15 rug to about 20 mg, about 20 mg to about 25 mg, about 25 mg to about 30 mg, about 30 mg to about 35 mg, about 35 mg to about 40 mg, about 40 trig to about 45 mg, or about 45 mg to about 50 mg of the active ingredient.

In some embodiments, the compositions provided herein contain from about 50 mg to about 500 mg of the active ingredient. One having ordinary skill in the art will appreciate that this embodies compounds or compositions containing about 50 mg to about 100 mg, about 100 mg to about 150 mg, about 150 mg to about 200 mg, about 200 mg to about 250 mg, about 250 mg to about 300 mg, about 350 mg to about 400 mg, or about 450 mg to about 500 rug of the active ingredient.

In some embodiments, the compositions provided herein contain from about 500 mug to about 1,000 mg of the active ingredient. One having ordinary skill in the art will appreciate that this embodies compounds or compositions containing about 500 mg to about 550 mg, about 550 mg to about 600 mg, about 600 mg to about 650 mg, about 650 mg to about 700 mg, about 700 mg to about 750 mg, about 750 mg to about 800 mg, about 800 mg to about 850 mg, about 850 mg to about 900 mg, about 900 mg to about 950 mg, or about 950 mg to about 1,000 mg of the active ingredient.

The active compound may be effective over a wide dosage range and is generally administered in a pharmaceutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms, and the like.

In some embodiments, the compound can be administered in an amount ranging from about 1 mg/kg to about 100 mg/kg. In some embodiments, the compound can be administered in an amount of about 1 mg/kg to about 20 mg/kg, about 5 mg/kg to about 50 mg/kg, about 10 mg/kg to about 40 mg/kg, about 15 mg/kg to about 45 mg/kg, about 20 mg/kg to about 60 mg/kg, or about 40 mg/kg to about 70 mg/kg. For example, about 5 mg/kg, about 10 mg/kg, about 15 mg/kg, about 20 mug/kg, about 25 mg/kg, about 30 mg/kg, about 35 mg/kg, about 40 mg/kg, about 45 mg/kg, about 50 mg/kg, about 55 mg/kg, about 60 mg/kg, about 65 mg/kg, about 70 mg/kg, about 75 mg/kg, about 80 mg/kg, about 85 mg/kg about 90 mg/kg, about 95 mg/kg, or about 100 mg/kg. In some embodiments, such administration can be once-daily or twice-daily (BID) administration.

The compounds of the present disclosure can also be used in combination with other compounds or known drugs that are used to treat cancer. Examples of the known drugs that can be used to treat lung cancer, thyroid cancer, pancreatic cancer, and breast cancer include, but are not limited to carboplatin, cisplatin, paclitaxel, capecitabine, gemcitabine, fluorouracil, everlolimus, doxorubicin, alectinib, afatinib, brigatinib, gefitinib, ceritinib, crizotinib, entrectinib, imatinib, lorlatinib, osimertinib, sorafenib, vandetanib, cabozantinib, erlotinib, sunitinib, alectinib, lenvatinib, trametinib, selpercatinib, and pralsetinib.

Provided is a pharmaceutical combination for treating cancer, for example, RET-altered cancer having one or more RET inhibitor resistance mutations, in a patient in need thereof, which comprises (i) a compound of formula (I) or (IA) or a pharmaceutically acceptable salt, hydrate, tautomer, or optical isomer thereof, (ii) an additional therapeutic agent, and (iii) optionally at least one pharmaceutically acceptable carrier, excipient, or diluent.

The term “pharmaceutical combination”, refers to a pharmaceutical therapy resulting from the mixing or combining of more than one active ingredient. For a combination, the compound of formula (I) or (IA) or a pharmaceutically acceptable salt, hydrate, tautomer, or optical isomer thereof and at least one additional therapeutic agent can be administered to a patient simultaneously or sequentially by the same or different route of administration in a single composition or two separate compositions to achieve the desired effect. The therapeutic agent can be administered in an amount to provide its desired therapeutic effect. The effective dosage range for each therapeutic agent is well known in the art, and the therapeutic agent is administered to a patient in need thereof within such established ranges.

### EXAMPLES

The following examples serve to illustrate the present disclosure. The examples are not intended to limit the scope of the claimed invention in any way.

### Assays:

KIF5B-RET mutation constructs, cell lines, and cell culture assays: BaF3 cells expressing KIF5B-RET, KIF5B-RET G810A, G810C, G810R, G810S, V804M were as reported in the art (British Journal of Pharmacology, 2018, 175, 3504-3515, Annals of Oncology, 2021, 32, 817-819, Annals of Oncology, 2021, 32, 261-268, Molecular Cancer Therapeutics, 2016, 15, 2521-2529. NPJ Precison Oncology. 2021, 5, 48, Journal of Biological Chemistry 2019, 294, 10428-10437). KIF5B-RET G810D and G810V mutations were generated by PCR-based mutagenesis with primers containing the desired mutations. BaF3 cells expressing these new KIF5B-RET mutants were established using a lentiviral vector, and the cell viability assay was performed using CellTiter-Glo reagent (Promega, Madison, WI) in 96-well plates (British Journal of Pharmacology, 2018, 175, 3504-3515). Drug screening and ICso data of BaF3/KR and mutation cells were from 2 or more experiments performed in 3 technical replicates.

### Animal Studies:

Animal experiments were approved by the Institutional Animal Care and Use Committee of the University of Oklahoma Health Sciences Center.

BaF3/KR(G810C) cells were tested free of 20 mouse pathogens (IDEXX, Columbia, MO) and mycoplasma. Cells (1×10⁷ cells/0.1 ml/each) were inoculated subcutaneously into the right flank of approximately 5-week old female SHO mice (Charles River, Wilmington, MA). After tumor formation, mice (6 mice/group) were treated with vehicle or test compounds at the indicated doses by oral gavage once a day. Tumor size and animal body weight were measured as described. Statistical analysis was performed using the unpaired Mann-Whitney test.

The alkynyl nicotinamides, compounds of formula (I) such as 5-((2-aminopyrimidin-5-yl)ethynyl)-N-(4-((4-methylpiperazin-1-yl)methyl)-3-(trifluoromethyl)phenyl)nicotinamide (Example 2), and the compounds of formula (IA) such as 5-((1H-pyrazolo[3,4-b]pyridin-5-yl)ethynyl)-N-(4-((4-methylpiperazin-1-yl)methyl)-3-(trifluoromethyl)phenyl)nicotinamide (Example 1), inhibit RET mutants, such as RET V804M and G810R and G810C better than ponatinib (an alkynyl benzamide). The results are shown in Table 1.

TABLE 1
Sensitivity of test compounds on RET V804M and G810 mutants determined in B/KR and mutant cell lines.

| | IC₅₀ of RET mutants (nM) | | | |
|---|---|---|---|---|
| Compound | WT | V804M | G810A | G810C |
| Example 1 | 1.70 ± 0.08 | 14.08 ± 0.54 | 1.56 ± 0.07 | 11.37 ± 0.32 |
| Example 2 | 3.49 ± 0.16 | 20.30 ± 1.09 | 3.89 ± 0.07 | 12.24 ± 0.63 |
| Ponatinib | 13.75 ± 1.19 | 111.5 ± 5.47 | 8.84 ± 0.21 | 50.33 ± 4.53 |
| LOXO292 | 14.96 ± 0.66 | 127.40 ± 6.42 | 180.10 ± 14.6 | 1371 ± 153.6 |
| BLU667 | 19.46 ± 1.00 | 23.75 ± 1.02 | 85.13 ± 3.26 | 956.50 ± 60.8 |
| | IC₅₀ of RET mutants (nM) | | | |
| Compound | G810D | G810R | G810S | G810V |
| Example 1 | 5.21 ± 0.36 | 24.53 ± 0.79 | 7.76 ± 0.33 | 4.10 ± 0.30 |
| Example 2 | 1.47 ± 0.05 | 46.75 ± 1.11 | 8.02 ± 0.21 | 8.34 ± 0.36 |
| Ponatinib | 10.10 ± 0.64 | 124.9 ± 9.39 | 33.94 ± 2.21 | 24.10 ± 9.45 |
| LOXO292 | 254.10 ± 11.5 | 2580 ± 97.26 | 940.30 ± 37.34 | 25.71 ± 0.76 |
| BLU667 | 499.90 ± 17.0 | 7939 ± 214.9 | 565.9 ± 17.71 | 53.19 ± 1.83 |

Compound of Example 2 inhibits hERG with IC₅₀ of 23 μM, whereas ponatinib (an alkynyl benzamide) inhibits hERG with IC₅₀ of ~1 μM. Compound of Example 2 had better drug-like properties than previously reported nicotinamide compounds, such as HSN608 (U.S. Pat. No. 11,001,559). For example, while HSN608 is 99% protein-bound (human serum), compound of Example 2 is 94% protein-bound. At pH 6.8, HSN608 has a solubility of 0.07 μM, while compound of Example 2 has a solubility of 117 μM. The more desirable drug-like properties of compound of Example 2 (compared to HSN608) translated into better in vivo efficacy.

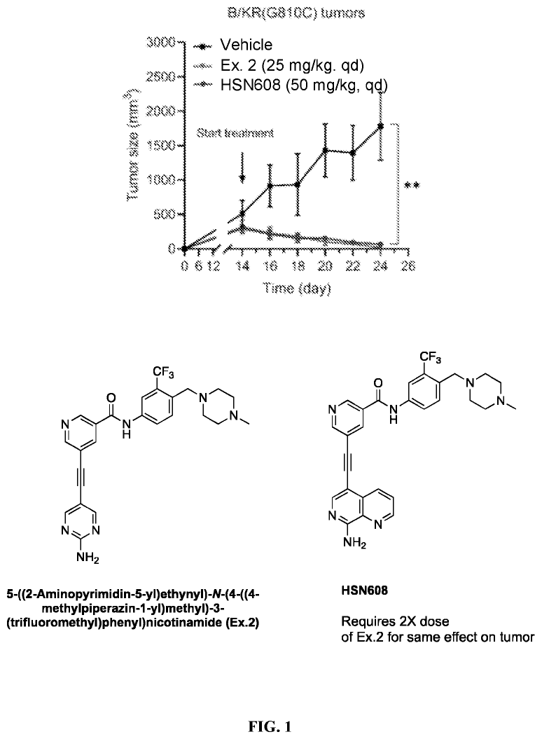

FIG. 1 shows that 25 mg/Kg dose of the compound of Example 2 was able to cause tumor regression of mice bearing B/KR(G810C) CDX tumors, whereas it required twice the dose 50 mg/Kg for HSN608.

Most kinase inhibitors also target other kinases in addition to the intended targets, so one skilled in the art could also utilize such compounds for the treatment of other cancers or for non-oncology disease states, for which kinases play prominent roles.

Sonogashira Coupling was performed using the procedure which is well known in the art (Bioorg Chem, 2019, 90, 103052), which is hereby specifically incorporated by reference for its teachings regarding the same.

### General Methods for Synthesis and Characterization of Compounds:

All reactions utilizing air- or moisture-sensitive reagents were performed in dried glassware under an atmosphere of argon (Ar), using commercially purchased solvents and reagents without further purification unless otherwise noted. ¹H NMR (500 MHz) and ¹³C NMR (126 MHz) spectra were recorded using a Bruker AV-III-500-HD NMR (Bruker, USA), Coupling constants were reported in Hertz, and peak shifts are reported in ((ppm) relative to CDCl₃ (¹H 7.26 ppm, ¹³C 77.16 ppm), CD₃OD (¹H 3.31 ppm. ¹³C 49.00 ppm), dimethyl sulfoxide (DMSO)-d6 (¹H 2.50 ppm. ¹³C 39.52 ppm) using Me₄Si as an internal standard. ¹H NMR data were reported as follows: chemical shift (δ ppm) (multiplicity, coupling constant (Hz), and integration). Multiplicities are reported as follows: s=singlet, d=doublet, t=triplet, q=quartet, m=multiplet, or combinations thereof. High-resolution mass spectroscopy (HRMS) spectra were recorded using the electron spray ionization (ESI) technique and TOF mass analysis on LTQ Orbitrap (Research Instrumentation Center in the Department of Chemistry at Purdue University). All the synthesized compounds were characterized using ¹H and ¹³C NMR and HRMS data.

### General Amide Coupling Procedure:

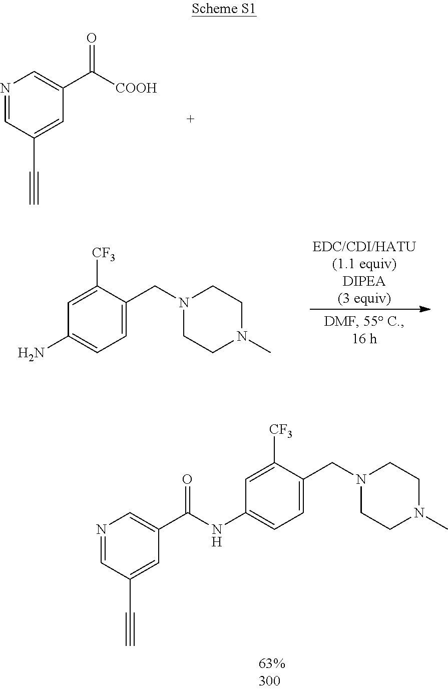 [SMILES: C#Cc1cncc(C(=O)C(=O)O)c1 ; CN1CCN(Cc2ccc(N)cc2C(F)(F)F)CC1 ; C#Cc1cncc(C(=O)Nc2ccc(CN3CCN(C)CC3)c(C(F)(F)F)c2)c1]

The round bottom flask was evacuated and back filled with Argon three times. Coupling reagent (4.95 mmol, 1.1 equiv.) was added to the two necked flask containing carboxylic acid (4.5 mmol, 1 equiv.) in a two-necked 50 mL round bottom flask in 10 mL DMF at room temperature. Diisopropylethylamine (10.35 mmol, 2.3 equiv.) was added, and the mixture was stirred for 1 hour at 0° C. Amine 1 (4.5 mmol, 1 equiv.) in 5 mL DMF was then added to the flask, and it was stirred overnight at room temperature. The reaction mixture was extracted in 30 mL ethyl acetate with washed with 10 mL water (3 times) followed by 10 mL brine. The combined organic layers were dried over anhydrous Na₂SO₄, filtered, and concentrated in vacuo. TLC:Rf 0.5 (2:8 MeOH:EtOAc). Column chromatography was performed using 1:9 (v/v) MeOH:EtOAc as mobile phase to isolate the product. Yield: 62%.

¹H NMR (500 MHz. DMSO) δ 10.70 (s, 1H), 9.07 (d, J=2.2 Hz, 1H), 8.86 (d, J=1.8 Hz, 1H), 8.41 (t, J=2.1 Hz, 1H), 8.17 (d, J=2.3 Hz, 1H), 8.00 (dd, J=8.5, 2.3 Hz, 1H), 7.71 (d, J=8.6 Hz, 1H), 4.58 (d, J=1.8 Hz, 1H), 3.55 (s, 2H), 2.38 (s, 5H), 2.32 (s, 3H), 2.14 (d, J=1.9 Hz, 3H);

¹³C NMR (126 MHz, DMSO) δ 163.81, 154.86, 148.91, 138.39, 138.24, 133.07, 131.83, 130.27, 123.99, 118.96, 85.67, 80.17, 57.91, 55.20, 53.18, 46.21.

### General Procedure for Synthesis of Pyrimidine Amine Analogues

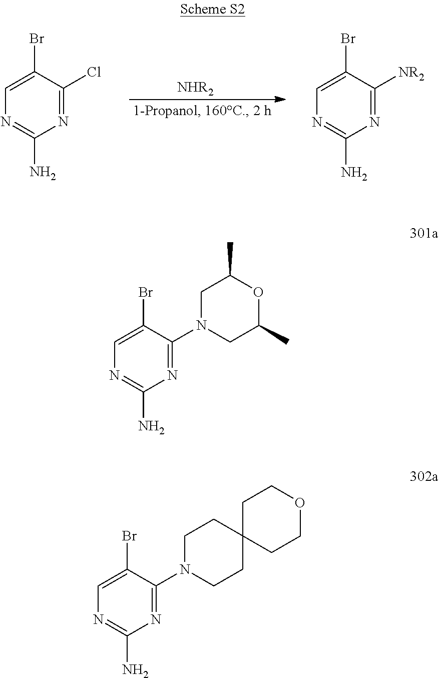

5-bromo-4-chloropyrimidin-2-amine (206 mg, 1 mmol) was dissolved in 1-propanol in a sealed vial. The mixture was treated with respective amine (1.5 equiv) and was heated at 160° C. for 2 h. The reaction was monitored by TLC. After completion of the reaction, the crude compound was extracted using EtOAc (3×20 mL). Combined organic layers were dried over anhydrous sodium sulphate, filtered, and concentrated in vacuo. Column chromatography was performed using 3:7 (v/v) EtOAc:Hexane as mobile phase to isolate the product.

Compound 301a: ¹H NMR (500 MHz, CDCl₃) δ 8.02 (s, 1H), 4.78 (s, 2H), 4.25-4.04 (m, 2H), 3.72 (dqd, J=10.5, 6.3, 2.2 Hz, 2H), 2.58 (dd, J=13.1, 10.4 Hz. 2H), 1.21 (d, J=6.3 Hz, 6H);

¹³C NMR (126 MHz, CDCl₃) δ 161.74, 161.08, 160.63, 95.08, 71.50, 52.98, 18.81.

Compound 302a: ¹H NMR (500 MHz, CDCl₃) δ 8.00 (s, 1H), 4.77 (s, 2H), 3.77-3.61 (m, 4H), 3.61-3.47 (m, 4H), 1.70-1.59 (m, 4H), 1.55 (t, J=5.4 Hz, 4H):

¹³C NMR (126 MHz, CDCl₃) δ 162.43, 161.11, 160.33, 95.27, 63.43, 43.24, 36.45, 35.73, 29.36.

### General Procedure for Synthesis of Pyrimidine Amine Analogues

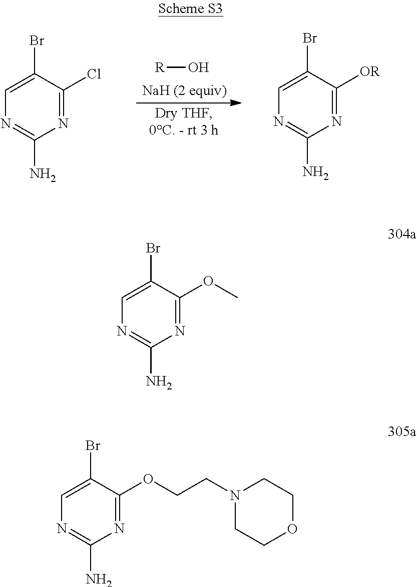

In a dry round bottom flask, aliphatic alcohol (2 mmol) was dissolved in dry THF (3 mL) under argon condition and was allowed to cool at 0° C. To this solution. NaH (60% in mineral oil) (2 equiv.) was added, and the reaction was allowed to cool at 0° C. for 30 mins. 5-bromo-4-chloropyrimidin-2-amine (206 mg, 1 mmol) was added to the above mixture in small portions, and the reaction was warmed to room temperature for 3 h. The reaction was monitored by TLC. After completion of the reaction, the reaction was quenched with water (2 mL). The crude compound was extracted using EtOAc (3×20 mL). Combined organic layers were dried over anhydrous sodium sulphate, filtered, and concentrated in vacuo. Column chromatography was performed using 3:7 (v/v) EtOAc:Hexane as mobile phase to isolate the product.

Compound 305-a: ¹H NMR (500 MHz, CDCl₃) δ 8.09 (s, 1H), 5.03 (s, 2H), 4.46 (t. J=5.8 Hz, 2H), 3.80-3.64 (m, 4H), 2.78 (t, J=5.9 Hz, 2H), 2.58 (dd, J=5.7, 3.7 Hz, 4H);

¹³C NMR (126 MHz, CDCl₃) δ 165.25, 161.61, 159.07, 93.93, 67.01, 65.11, 56.87, 54.08.

### General Procedure for Synthesis of Pyridyl Urea Analogues

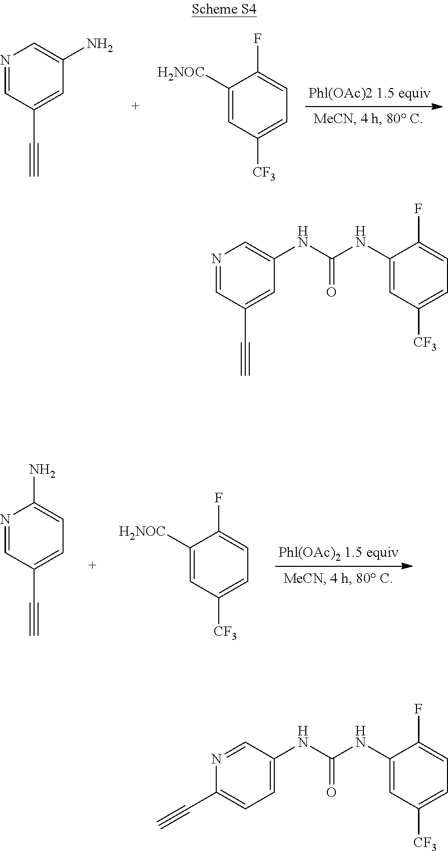

An oven-dried screw cap vial equipped with a magnetic stirrer bar was charged with aryl carboxamides (0.5 mmol, 1 equiv.), aminopyridines (1 equiv.) and PIDA (1.5 equiv.). MeCN (2 mL) was added via a syringe. The tube was sealed, and the mixture was allowed to stir at 80° C. for 4 h. The reaction was monitored using TLC. After completion of the reaction, the reaction mixture was allowed to cool to room temperature and then diluted with excess ethyl acetate. The organic layer was washed with brine, dried over Na₂SO₄, concentrated under reduced pressure, and purified by column chromatography [100-200 #silica, EtOAc:hexane=2:8] to give the desired product.

### General Procedure for Sonogashira Coupling

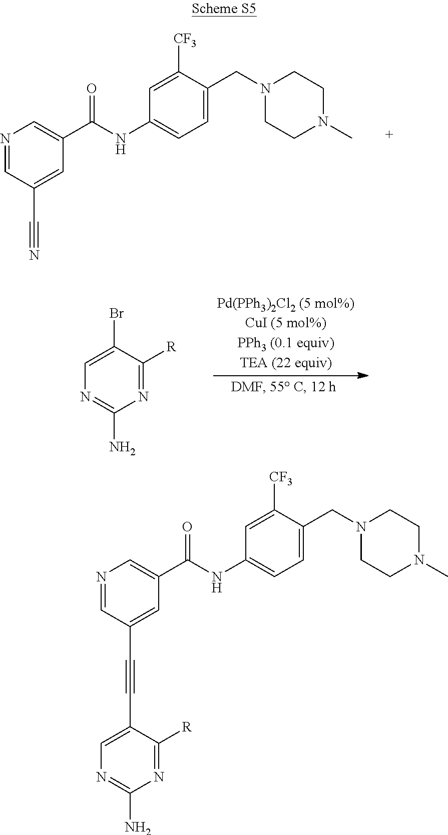

A solution of bromo compound (1 equiv, 0.5 mmol), Pd(PPh₃)₂Cl₂ (5 mol %), CuI (5 mol %) and triphenylphosphine (0.1 equiv) in triethylamine (22 equiv) was de-oxygenated using steam of argon gas. A de-oxygenated solution of alkyne (1.2 equiv) in DMF (3 mL) was added slowly over a period of 10 min to the solution and the reaction temperature was increased to 50° C. and allowed to stir 12 h. The reaction was quenched by the addition of NH₄Cl (5 mL) at room temperature. The crude compound was extracted using EtOAc (3×40 mL). Combined organic layers were dried over anhydrous sodium sulphate, filtered, and concentrated in vacuo. The pure product was then obtained via column chromatography. Yield=35-40%; TLC Rf=0.2 (10% MeOH/EtOAc).

### Example 1

### 5-((1H-Pyrazolo[3,4-b]pyridin-5-yl)ethynyl)-N-(4-((4-methylpiperazin-1-yl)methyl)-3-(trifluoromethyl)phenyl)nicotinamide

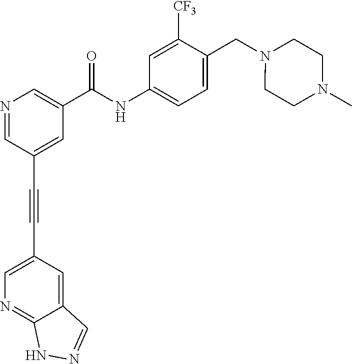 [SMILES: CN1CCN(Cc2ccc(NC(=O)c3cncc(C#Cc4cnc5[nH]ncc5c4)c3)cc2C(F)(F)F)CC1]

A solution of 5-bromo-1H-pyrazolo[3,4-b]pyridine (1 mmol), Pd(PPh₃)₄ (10 mol %), CuI (5 mol %), and triphenylphosphine (10 mg) in triethylamine (30 equiv) was de-oxygenated using steam of argon gas. Followed by the addition of a de-oxygenated solution of 5-ethynyl-N-(4-((4-methylpiperazin-1-yl)methyl)-3-(trifluoromethyl)phenyl)nicotinamide substrate (0.95 mmol) in DMF (4 mL) slowly over a period of 10 min. Reaction was allowed to stir at 55° C. for 12 h. After completion reaction was quenched by addition of NH₄Cl (5 mL) at room temperature and diluted with ethyl acetate (300 mL). The organic layer was extracted with water (2×50 mL) and washed with brine solution (1×50 mL). Combined organic layers were dried over anhydrous sodium sulphate, filtered, and concentrated in vacuo. Purified by flash column chromatography using dichloromethane/methanol (97:3 to 95:5).
Off-White Solid (53%);

¹H NMR (500 MHz, DMSO-d₆) δ 10.82 (s, 1H), 9.09 (d, J=2.1 Hz, 1H), 8.98 (d, J=2.0 Hz, 1H), 8.74 (d, J=2.0 Hz, 1H), 8.54 (dd, J=3.8, 2.2 Hz, 2H), 8.27-8.17 (m, 2H), 8.07 (dd, J=8.5, 2.2 Hz, 1H), 7.72 (d, J=8.5 Hz, 1H), 3.65 (s, 2H), 3.55-3.37 (bs, 8H), 2.69 (s, 3H);

¹³C NMR (126 MHz, DMSO-d₆) δ 163.9, 154.4, 151.6, 151.0, 148.6, 138.6, 137.9, 134.4, 133.9, 132.0, 130.2, 128.2 (q, J=30.2 Hz), 125.7 (q, J=274.6 Hz), 124.0, 121.0, 119.5, 117.8, 114.4, 111.5, 91.8, 86.8, 57.0, 53.3, 50.1, 42.9;

HRMS (ESI) m/z calcd for C₃₀H₃₀F₃N₈O [M+H]⁺ 575.2494, found 575.2489.

### Example 2

### 5-((2-Aminopyrimidin-5-yl)ethynyl)-N-(4-((4-methylpiperazin-1-yl)methyl)-3-(trifluoromethyl)phenyl)nicotinamide

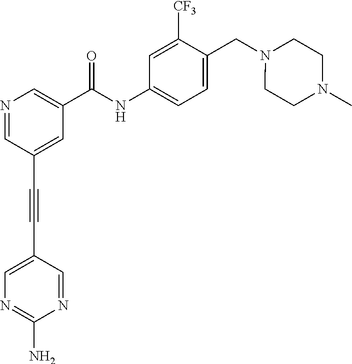 [SMILES: CN1CCN(Cc2ccc(NC(=O)c3cncc(C#Cc4cnc(N)nc4)c3)cc2C(F)(F)F)CC1]

To a vial with 5-bromopyrimidin-2-amine (70 mg, 0.402 mmol, 1 equiv), Pd(PPh₃)₂Cl₂ (10 mol %), CuI, PPh₃ anhydrous TEA (1.5 mL) was added. The solution was then degassed with a stream of argon gas for 10 minutes. A solution of 5-ethynyl-N-(4-((4-methylpiperazin-1-yl)methyl)-3-(trifluoromethyl)phenyl)nicotinamide (197.1 mg, 0.482 mmol, 1.2 equiv) in anhydrous degasses DMF (3 mL) was then slowly added over the course of 10 minutes. Once the addition was complete, it was then moved to 55° C. and allowed to run overnight. The reaction was then cooled to room temperature and concentrated under reduced pressure. The pure product was then obtained via column chromatography.

Off-white solid (30.2%).

¹H NMR (500 MHz, DMSO-d₆) δ 10.77 (s, 1H), 9.05 (d, J=2.1 Hz. 1H), 8.87 (d, J=2.0 Hz, 1H), 8.47 (s, 2H), 8.44 (t, J=2.1 Hz, 1H), 8.19 (d, J=2.2 Hz, 1H), 8.03 (dd, J=8.6, 2.2 Hz, 1H), 7.71 (d, J=8.5 Hz, 1H), 7.25 (s, 2H), 3.57 (s, 2H), 2.41 (s, 8H), 2.21 (s, 3H);

¹³C NMR (125 MHz, DMSO-d₆) δ 163.8, 154.8, 148.9, 148.9, 139.8, 138.3, 134.9, 132.5, 131.9, 130.2, 128.1, 127.8 (q, J=28.7 Hz), 125.7 (q, J=272.5 Hz), 123.9, 119.8, 118.9, 117.7, 85.6, 80.1, 57.4, 54.2, 51.6, 44.6;

HRMS (ESI) m/z calcd for C₂₅H₂₅F₃N₇O [M+H]⁺ 496.2072, found 496.2075.

### Example 3

### 5-((3-Methyl-1H-pyrazolo[3,4-b]pyridin-5-yl)ethynyl)-N-(4-((4-methylpiperazin-1-yl)methyl)-3-(trifluoromethyl)phenyl)nicotinamide

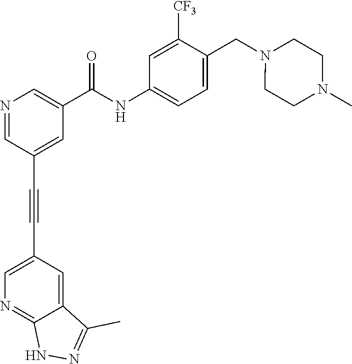 [SMILES: Cc1n[nH]c2ncc(C#Cc3cncc(C(=O)Nc4ccc(CN5CCN(C)CC5)c(C(F)(F)F)c4)c3)cc12]

In a 25 mL round bottom flask containing 5-bromo-3-methyl-1H-pyrazolo[3,4-b]pyridine (1 mmol), 5-ethynyl-N-(4-((4-methylpiperazin-1-yl)methyl)-3-(trifluoromethyl)phenyl)nicotinamide (1.1 mmol), PdCl₂(PPh₃)₂ (3 mol %), XPhos (2 mol %), Cs₂CO₃ (3 equiv) and CuI (1 mol %), anhydrous DMF (5 mL) and DIPEA (2.5 mL) was added under inert condition. The reaction mixture was allowed to stir at 60° C. for overnight. After the completion, the reaction mixture was concentrated and extracted with ethyl acetate. The organic layer washed with 2 N NaOH aqueous solution (20 mL) and brine solution (30 mL). The organic layer was passed through celite bad. The collected organic layer was dried with sodium sulfate, concentrated, and purified via silica gel column chromatography to yield the desired product using DCM/MeOH (95:5) as a solvent system.

Off-white solid, Yield: 33%.

¹H NMR (500 MHz, DMSO-d₆) δ 10.85 (s, 1H), 9.10 (d, J=2.2 Hz, 1H), 8.96 (d. J=2.0 Hz, 1H), 8.69 (d, J=1.9 Hz, 1H), 8.53 (dt, J=4.8, 2.3 Hz, 2H), 8.22 (d, J=2.4 Hz, 1H), 8.09 (dd, J=8.7, 2.3 Hz, 1H), 7.72 (d, J=8.5 Hz, 1H), 3.66 (s, 2H), 3.29-2.79 (m, 8H), 2.71 (s, 3H), 2.52 (s, 3H);

¹³C NMR (126 MHz, DMSO-d₆) δ 163.97, 154.37, 151.75, 151.43, 148.59, 142.39, 138.65, 137.87, 133.56, 133.56, 132.06, 130.29, 127.99 (q, J=28.98 Hz), 125.78 (q, J=273.4 Hz), 124.02, 119.64, 117.88, 114.07, 110.58, 92.06, 86.67, 57.08, 53.24, 49.96, 12.61.

### Example 4

### 5-((2-amino-4-((2S,6R)-2,6-dimethylmorpholino)pyrimidin-5-yl)ethynyl)-N-(4-((4-methylpiperazin-1-yl)methyl)-3-(trifluoromethyl)phenyl)nicotinamide

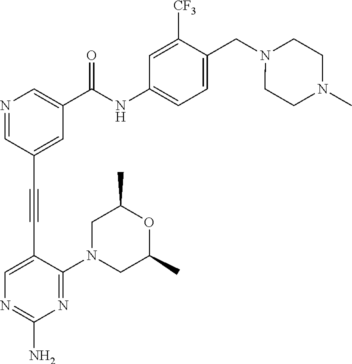 [SMILES: C[C@@H]1CN(c2nc(N)ncc2C#Cc2cncc(C(=O)Nc3ccc(CN4CCN(C)CC4)c(C(F)(F)F)c3)c2)C[C@H](C)O1]

¹H NMR (500 MHz, DMSO) δ 10.75 (s, 1H), 9.01 (d, J=2.1 Hz, 1H), 8.78 (d, J=2.0 Hz, 1H), 8.30 (t, J=2.1 Hz, 1H), 8.21-8.10 (m, 2H), 8.01 (dd, J=8.4, 2.3 Hz, 1H), 7.71 (d, J=8.5 Hz, 1H), 6.76 (s, 2H), 4.75-4.62 (m, 2H), 3.64 (dqd, J=12.5, 6.2, 2.1 Hz, 2H), 3.55 (s, 3H), 2.61 (dd, J=13.2, 10.5 Hz, 2H), 2.37 (s, 8H), 2.14 (s, 4H), 1.11 (d, J=6.2 Hz, 7H):

¹³C NMR (126 MHz, DMSO) δ 164.1, 164.3, 153.2, 147.6, 138.3, 136.7, 133.0, 131.8, 130.4, 128.1, 125.8, 120.6, 117.7, 92.2, 91.4, 90.1, 71.5, 57.9, 55.2, 53.2, 51.7, 46.2, 19.1;

HRMS (ESI) m/z calcd for C₃₁H₃₅F₃N₈O₂[M+H]⁺ 608.2835, found 608.2830.

### Example 5

### 5-((2-amino-4-(3-oxa-9-azaspiro[5.5]undecan-9-yl)pyrimidin-5-yl)ethynyl)-N-(4-((4-methylpiperazin-1-yl)methyl)-3-(trifluoromethyl)phenyl)nicotinamide

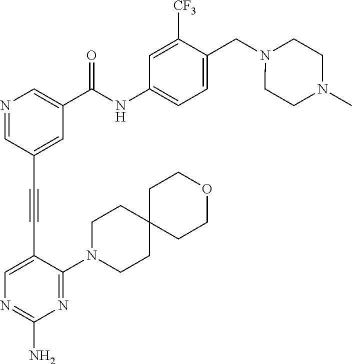 [SMILES: CN1CCN(Cc2ccc(NC(=O)c3cncc(C#Cc4cnc(N)nc4N4CCC5(CCOCC5)CC4)c3)cc2C(F)(F)F)CC1]

¹H NMR (500 MHz, DMSO) δ 10.78 (s, 1H), 8.99 (d, J=2.2 Hz, 1H), 8.79 (d, J=2.1 Hz, 1H), 8.31 (t, J=2.2 Hz, 1H), 8.18 (d, J=2.2 Hz, 1H), 8.10 (s, 1H), 8.02 (dd, J=8.5, 2.2 Hz. 1H), 7.71 (d, J=8.5 Hz, 1H), 6.64 (s, 2H), 3.93-3.82 (m, 4H), 3.60-3.51 (m, 6H), 2.46-2.20 (m, 8H), 2.14 (s, 3H), 1.64-1.53 (m, 4H), 1.47 (t, J=5.3 Hz, 4H):

¹³C NMR (126 MHz, DMSO) δ 164.1, 162.3, 157.5, 156.5, 154.9, 153.5, 147.5, 138.3, 132.9, 131.8, 123.9, 121.2, 117.9, 92.6, 90.9, 89.9, 62.9, 57.9, 55.2, 53.2, 46.2, 36.4, 35.9, 29.9;

HRMS (ESI) m/z calcd for C₃₄H₃₉F₃N₈O₂ [M+H]⁺ 648.3148, found 648.3129.

### Example 6

### 5-((2-amino-4-methoxypyrimidin-5-yl)ethynyl)-N-(4-((4-methylpiperazin-1-yl)methyl)-3-(trifluoromethyl)phenyl)nicotinamide

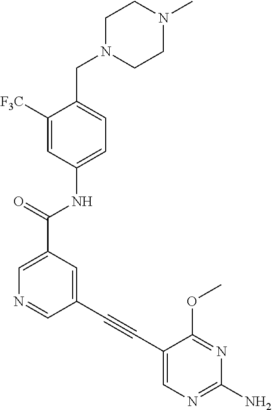 [SMILES: COc1nc(N)ncc1C#Cc1cncc(C(=O)Nc2ccc(CN3CCN(C)CC3)c(C(F)(F)F)c2)c1]

¹H NMR (500 MHz, DMSO) δ 10.81 (d, J 2.8 Hz, 1H), 9.17 (d, J=2.1 Hz, 1H), 8.99 (d, J=2.0 Hz, 1H), 8.55 (d, J=2.2 Hz, 1H), 8.50 (s, 1H), 8.19 (d, J=2.2 Hz, 1H), 8.05-7.99 (m, 1H), 7.77-7.70 (m, 2H), 7.12 (s, 2H), 3.56 (s, 3H), 2.37 (s, 9H), 2.16 (s, 4H), 1.89 (s. 1H);

HRMS (ESI) m/z calcd for C₂₆H₂₆F₃N₇O₂[M+H]⁺ 525.2100, found 525.2096.

### Example 7

### 5-((2-amino-4-(2-morpholinoethoxy)pyrimidin-5-yl)ethynyl)-N-(4-((4-methylpiperazin-1-yl)methyl)-3-(trifluoromethyl)phenyl)nicotinamide

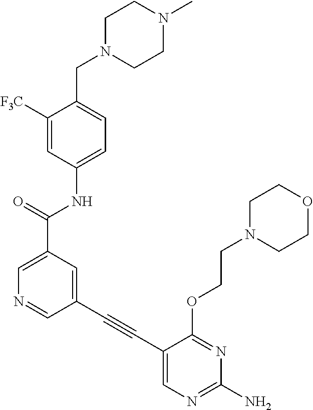 [SMILES: CN1CCN(Cc2ccc(NC(=O)c3cncc(C#Cc4cnc(N)nc4OCCN4CCOCC4)c3)cc2C(F)(F)F)CC1]

¹H NMR (500 MHz, MeOD) δ 9.01 (d, J=2.2 Hz, 1H), 8.78 (d, J=2.0 Hz, 1H), 8.37 (t, J=2.1 Hz, 1H), 8.21 (s, 1H), 8.14 (d, J=2.3 Hz, 1H), 7.94 (d, J=2.3 Hz, 1H), 7.92-7.87 (m, 1H), 4.59 (t, J=5.5 Hz, 3H), 3.70-3.64 (m, 7H), 2.84 (t, J=5.5 Hz, 2H), 2.66 (t, J=4.7 Hz, 5H), 2.33 (s, 4H), 1.90 (s, 8H);

HRMS (ESI) m/z calcd for C₃₁H₃₅F₃N₈O₃[M+H]⁺ 624.2784, found 624.2774.

### Example 8

### 5-((2-aminopyrimidin-5-yl)ethynyl)-N-(5-(trifluoromethyl)-1H-pyrazol-3-yl)nicotinamide

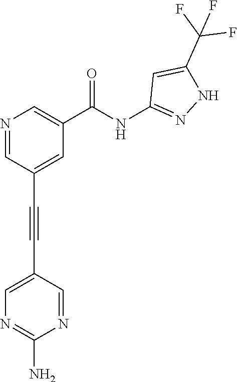 [SMILES: Nc1ncc(C#Cc2cncc(C(=O)Nc3cc(C(F)(F)F)[nH]n3)c2)cn1]

¹H NMR (500 MHz, DMSO) δ 11.51 (s, 1H), 9.05 (s, 1H), 8.90 (s, 1H), 8.48 (s, 2H), 8.41 (s, 1H), 7.26 (s. 2H), 6.49 (s, 1H);

¹³C NMR (126 MHz, DMSO) δ 162.66, 161.08, 154.48, 148.24, 137.48, 105.35, 93.50, 88.33; HRMS (ESI) m/z calcd for C₁₆H₁₀F₃N₇O [M+H]⁺ 373.0899, found 373.0891.

### Example 9

### 5-((2-aminopyrimidin-5-yl)ethynyl)-N-(5-(tert-butyl)isoxazol-3-yl)nicotinamide

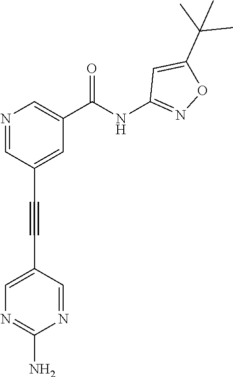 [SMILES: CC(C)(C)c1cc(NC(=O)c2cncc(C#Cc3cnc(N)nc3)c2)no1]

¹H NMR (500 MHz, MeOD) δ 12.43 (s, 1H), 9.87 (s, 1H), 9.69 (s, 1H), 9.27 (d, J=16.9 Hz, 3H), 8.06 (s. 2H), 7.53 (s, 1H), 2.12 (s, 9H);

¹³C NMR (126 MHz, MeOD) δ 181.95, 164.54, 163.45, 161.86, 159.28, 155.13, 149.18, 138.70, 106.23, 95.06, 90.57, 89.22, 33.86, 29.65;

HRMS (ESI) m/z calcd for C₁₉H₁₈N₆O₂ [M+H]⁺ 362.1491, found 362.1485.

### Example 10

### 5-((2-aminopyrimidin-5-yl)ethynyl)-N-(5-(tert-butyl)-1H-pyrazol-3-yl)nicotinamide

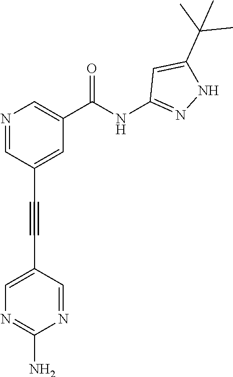 [SMILES: CC(C)(C)c1cc(NC(=O)c2cncc(C#Cc3cnc(N)nc3)c2)n[nH]1]

¹H NMR (500 MHz, DMSO) δ 12.21 (s, 1H), 11.05 (s, 1H), 9.12 (d, J=2.1 Hz, 1H), 8.97 (d, J=2.0 Hz. 1H), 8.54 (d, J=2.3 Hz. 1H), 6.43 (s, 1H), 1.27 (s, 9H);

¹³C NMR (126 MHz, DMSO) δ 162.18, 155.24, 149.59, 139.32, 130.21, 117.63, 93.72, 79.82, 76.76, 40.49, 31.21, 30.43;

HRMS (ESI) m/z calcd for C₁₉H₁₉N₇O [M+H]⁺ 361.1651, found 361.1650.

### Example 11

### 1-(5-((2-aminopyrimidin-5-yl)ethynyl)pyridin-3-yl)-3-(2-fluoro-5-(trifluoromethyl)phenyl)urea

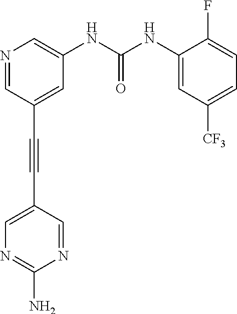 [SMILES: Nc1ncc(C#Cc2cncc(NC(=O)Nc3cc(C(F)(F)F)ccc3F)c2)cn1]

¹H NMR (500 MHz, DMSO) δ 9.48 (s, 1H), 9.11 (s, 1H), 8.70-8.42 (m, 3H), 8.22 (s, 1H), 7.69-7.33 (m, 5H).

### Example 12

### 5-((5-aminopyrazin-2-yl)ethynyl)-N-(5-(tert-butyl)isoxazol-3-yl)nicotinamide

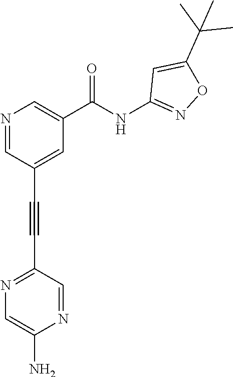 [SMILES: CC(C)(C)c1cc(NC(=O)c2cncc(C#Cc3cnc(N)cn3)c2)no1]

¹H NMR (500 MHz, MeOD) δ 9.03 (s, 1H), 8.87 (s, 1H), 8.45 (t, J=2.0 Hz, 1H), 8.19 (s, 1H), 7.93 (s, 1H), 7.68-7.60 (m, 1H), 7.55 (td, J=7.6, 3.0 Hz, 1H), 7.30 (s, 1H), 6.70 (s, 1H), 1.37 (s, 9H).

### Example 13

### 5-((5-aminopyrazin-2-yl)ethynyl)-N-(5-(tert-butyl)-1H-pyrazol-3-yl)nicotinamide

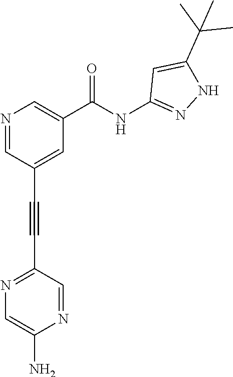 [SMILES: CC(C)(C)c1cc(NC(=O)c2cncc(C#Cc3cnc(N)cn3)c2)n[nH]1]

¹H NMR (500 MHz, MeOD) δ 9.06 (d, J=33.3 Hz, 1H), 8.87 (d, J=21.1 Hz, 1H), 8.47 (d, J=23.0 Hz, 1H), 6.47 (s, 1H), 1.35 (s, 9H).

### Example 14

### 5-((5-aminopyrazin-2-yl)ethynyl)-N-(5-(trifluoromethyl)-1H-pyrazol-3-yl)nicotinamide

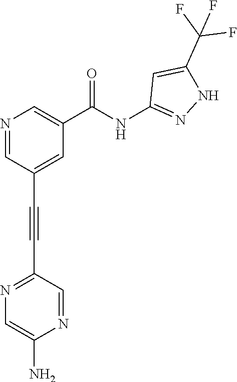 [SMILES: Nc1cnc(C#Cc2cncc(C(=O)Nc3cc(C(F)(F)F)[nH]n3)c2)cn1]

¹H NMR (500 MHz, MeOD) δ 9.07 (d, J=2.1 Hz, 1H), 8.92-8.86 (m, 1H), 8.48 (t, J=2.1 Hz, 1H), 8.20 (d, J=1.5 Hz, 1H), 7.94 (d, J=1.5 Hz, 1H), 7.77-7.48 (m, 1H), 6.41 (s, 2H).

### Example 15

### 1-(5-((5-aminopyrazin-2-yl)ethynyl)pyridin-3-yl)-3-(2-fluoro-5-(trifluoromethyl)phenyl)urea

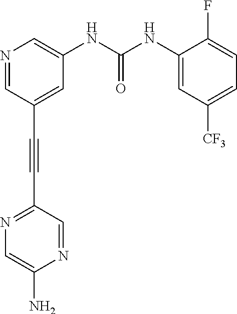 [SMILES: Nc1cnc(C#Cc2cncc(NC(=O)Nc3cc(C(F)(F)F)ccc3F)c2)cn1]

¹H NMR (500 MHz, DMSO) δ 10.66 (s, 1H), 10.09 (s, 1H), 8.63 (dd, J=7.2, 2.4 Hz, 1H), 8.44 (dd, J=2.3, 0.8 Hz, 1H), 8.15-8.12 (m, 1H), 7.91 (dd, J=8.7, 2.3 Hz. 1H), 7.88-7.85 (m, 1H), 7.60 (dddt, J=8.4, 6.7, 3.9, 2.6 Hz, 1H), 7.56-7.50 (m, 1H), 7.46-7.41 (m, 1H), 6.92 (s, 2H).

As used herein, the following terms and phrases shall have the meanings set forth below. Unless defined otherwise, all technical and scientific terms used herein have the same meaning as commonly understood to one of ordinary skill in the art.

The term “about” can allow for a degree of variability in a value or range, for example, within 10%, within 5%, or within 1% of a stated value or of a stated limit of a range.

The term “substantially” can allow for a degree of variability in a value or range, for example, within 90%, within 95%, or within 99% of a stated value or of a stated limit of a range.

The terms “a,” “an,” or “the” are used to include one or more than one unless the context clearly dictates otherwise. The term “or” is used to refer to a nonexclusive “or” unless otherwise indicated. In addition, the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. The terms “including” and “having” are defined as comprising (i.e., open language).

It will be appreciated by persons skilled in the art that the present disclosure is not limited by what has been particularly shown and described herein above. Rather the scope of the present disclosure includes both combinations and sub-combinations of the various features described hereinabove as well as variations and modifications which would occur to persons skilled in the art upon reading the specification and which are not in the prior art.

All patents, patent application publications, journal articles, textbooks, and other publications mentioned in the specification are indicative of the level of skill of those in the art to which the disclosure pertains. All such publications are incorporated herein by reference to the same extent as if each individual publication were specifically and individually indicated to be incorporated by reference.

1. A compound of formula (I), or a pharmaceutically acceptable salt, hydrate, tautomer, or optical isomer thereof: 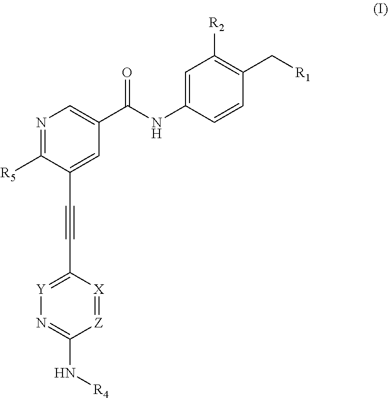
wherein
R₁ is NRₐR_{b}, wherein Rₐ and R_{b} each independently represents a group selected from H, alkyl, cycloalkyl, heterocycloalkyl, aryl, heteroaryl, —(CH₂)ₚ-alkyl, —(CH₂)ₚ-cycloalkyl, —(CH₂)ₚ-heterocycloalkyl, —(CH₂)ₚ—SO₂R_{c}, —(CH₂)ₚ—OR_{c}, and —(CH₂)ₚ—NRₐR_{b}, wherein Rₐ and R_{b} each independently is optionally substituted with at least one of alkyl, halo, OR_{c}, and NH₂; or
Rₐ and R_{b}, together with the nitrogen atom to which they are attached, form a 4- to 8-membered heterocycloalkyl group, which is a mono or a bicyclic ring, either of which is optionally substituted with a group selected from alkyl, cycloalkyl, halo, nitro, CN, SO₂R_{c}, OR_{d}, —(CH₂)ₚ—OR_{c} and NR_{c}R_{c};
R_{c} is H or alkyl;
R_{d} is H, halo, OH, CN, alkyl, —(CH₂)ₚ-alkyl, —(CH₂)ₚ-cycloalkyl, —(CH₂)ₚ-heterocycloalkyl, —(CH₂)ₚ—SO₂R_{d}, —(CH₂)ₚ—OR_{d}, —(CH₂)ₚ—(OR_{d})₂, or —(CH₂)ₚ—NRₐR_{b};
p is 0-5;
R₂ represents a group independently selected from alkyl, CF₃, OCF₃, and halo;
X, Y, and Z each independently represent a group selected from CR₃ and N with the proviso that the ring of which X, Y, and Z is comprised contains at least two N atoms, wherein R₃ is H, alkyl, ORₐ, or NRₐR_{b}, wherein Rₐ and R_{b} are as defined above;
R₄ represents a group selected from H, halo, OH, CN, alkyl, —(CH₂)ₚ-alkyl, —(CH₂)ₚ-cycloalkyl, —(CH₂)ₚ-heterocycloalkyl, —(CH₂)ₚ—SO₂R_{d}, —(CH₂)ₚ—OR_{d}, —(CH₂)ₚ—(OR_{d})₂, and —(CH₂)ₚ—NRₐR_{b}, wherein Rₐ, R_{b}, R_{d}, and p are as defined above; and
R₅ represents a group independently selected from H, alkyl, and halo.

2. The compound of claim 1, wherein each of X, Y, and Z is independently a group selected from CR₃ and N with the proviso that the ring of which X, Y, and Z is comprised contains at least two N atoms, wherein R₃ represents a group selected from

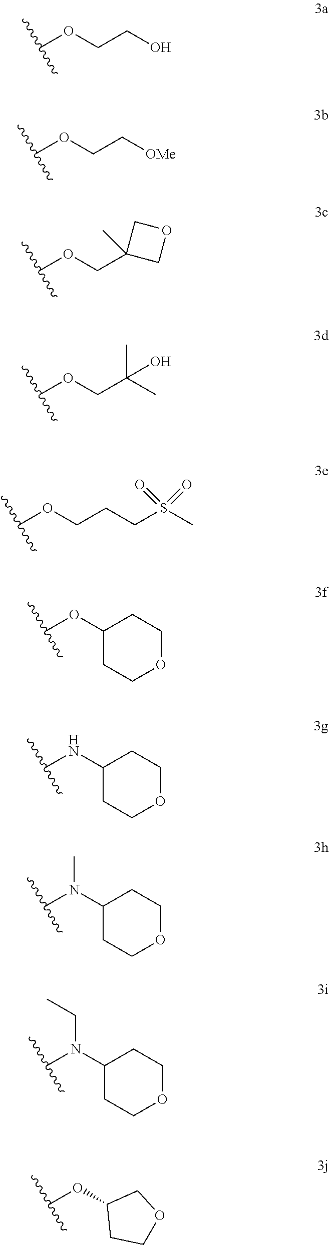

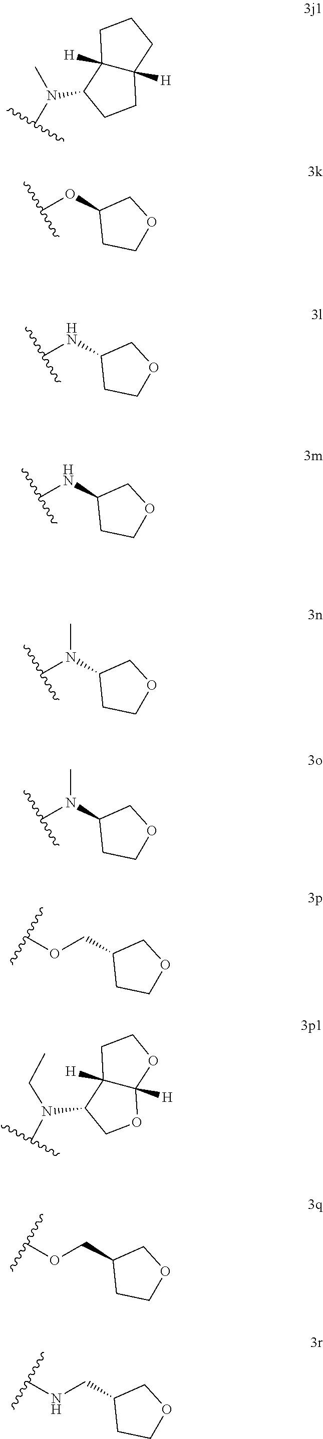

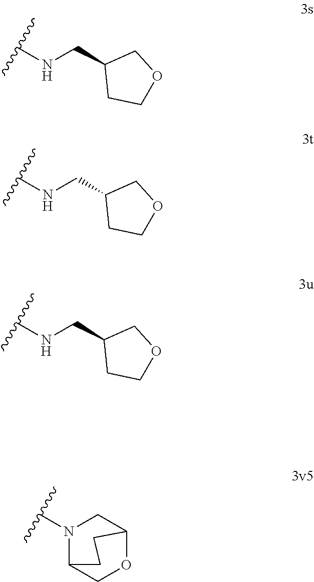 [SMILES: *NC[C@@H]1CCOC1 ; *NC[C@H]1CCOC1 ; *NC[C@@H]1CCOC1 ; *N1CC2CCC1CO2]

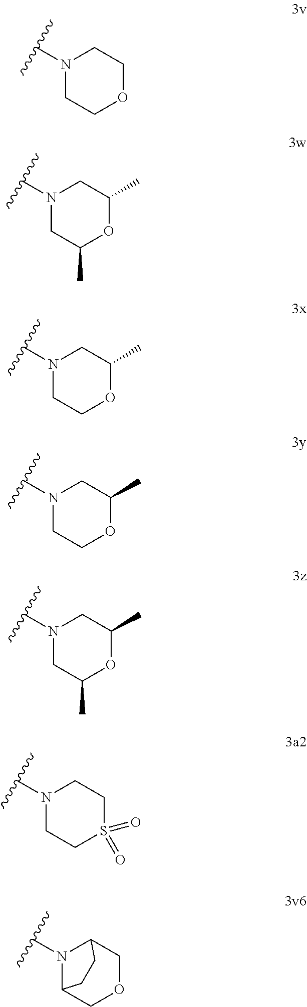

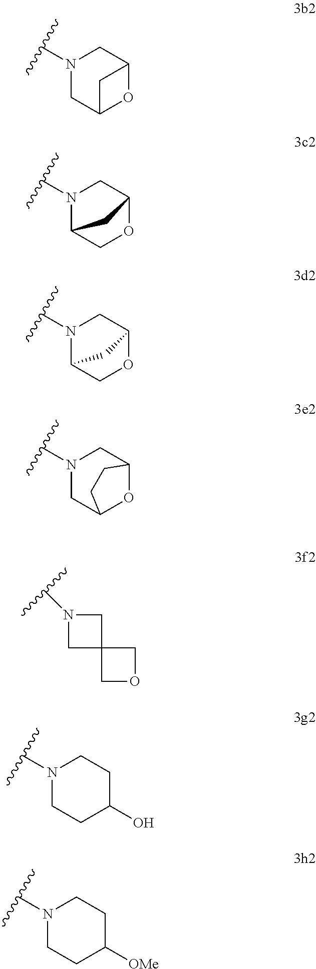

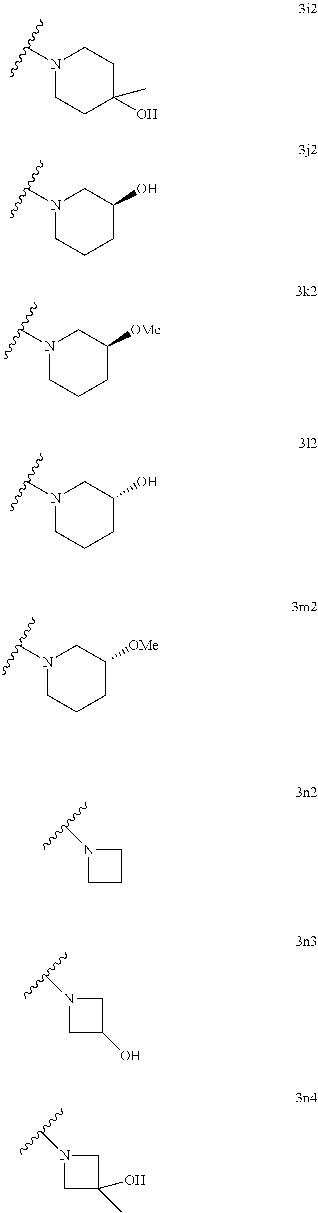

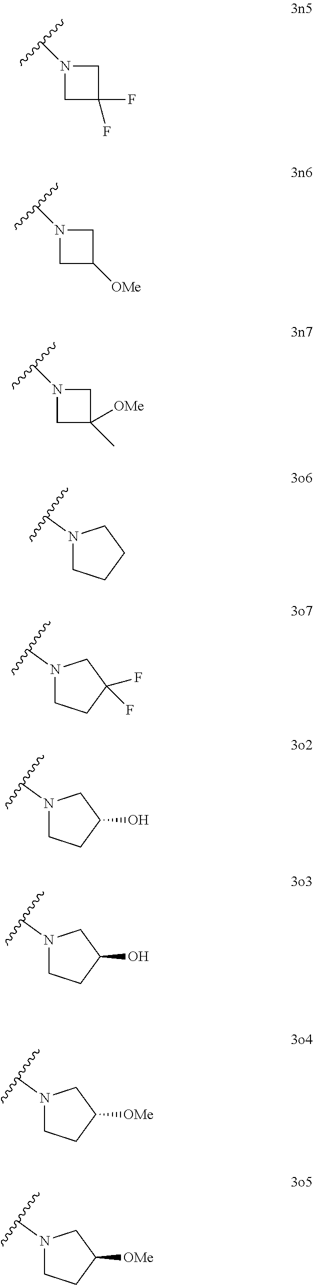

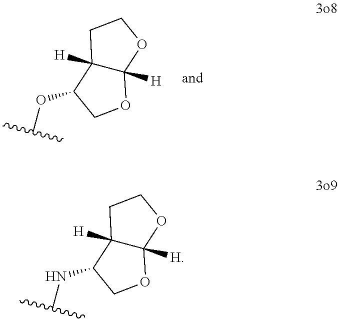

3. The compound of claim 1, wherein R₄ is alkyl or a group selected from

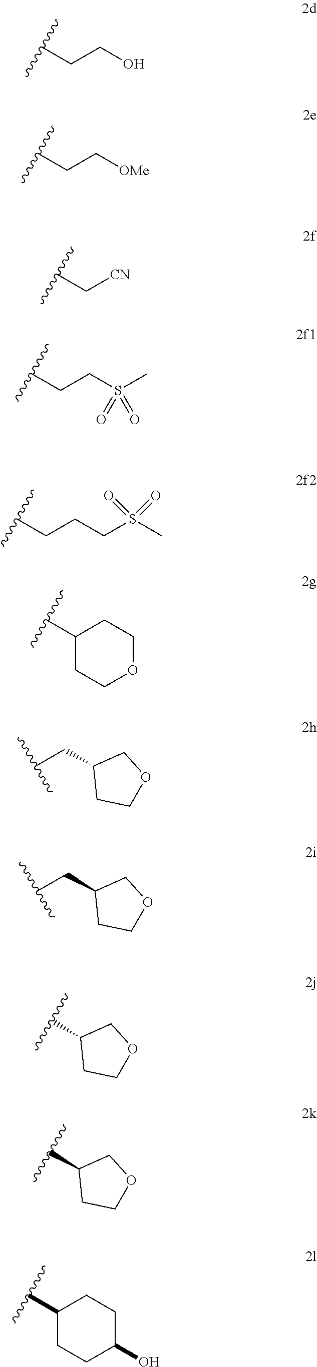

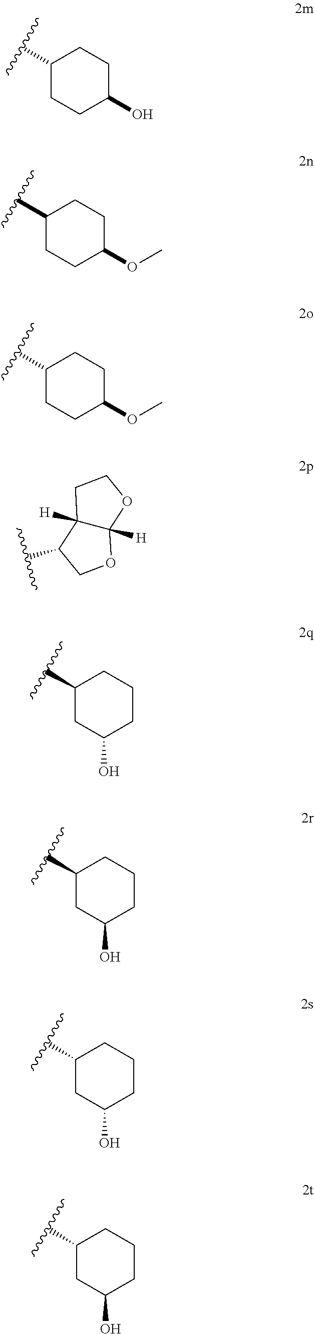

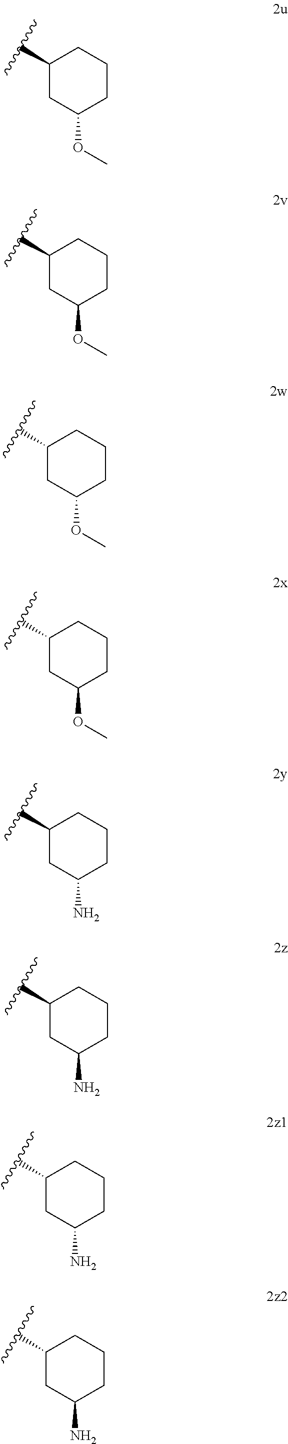

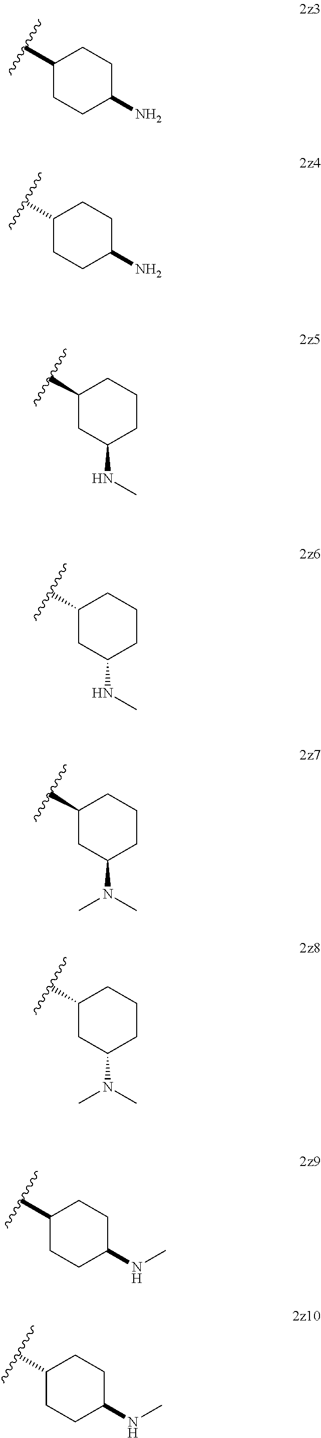

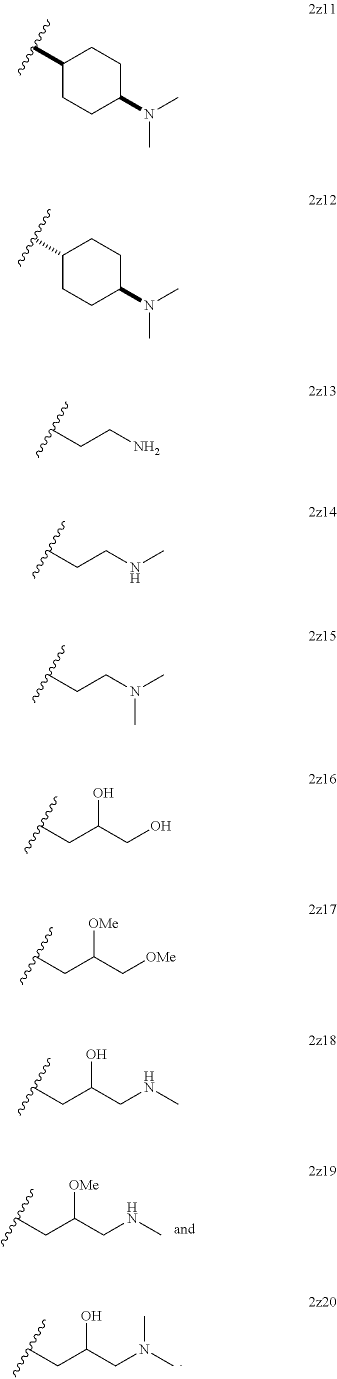

4. The compound of claim 1, wherein the compound is 5-((2-aminopyrimidin-5-yl)ethynyl)-N-(4-((4-methylpiperazin-1-yl)methyl)-3-(trifluoromethyl)phenyl)nicotinamide, 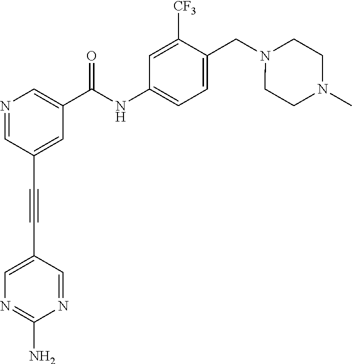 [SMILES: CN1CCN(Cc2ccc(NC(=O)c3cncc(C#Cc4cnc(N)nc4)c3)cc2C(F)(F)F)CC1]
or a pharmaceutically acceptable salt, hydrate, tautomer, or optical isomer thereof.

5. The compound of claim 1, wherein the compound is 5-((3-amino-1,2,4-triazin-5-yl)ethynyl)-N-(4-((4-methylpiperazin-1-yl)methyl)-3-(trifluoromethyl)phenyl)nicotinamide, 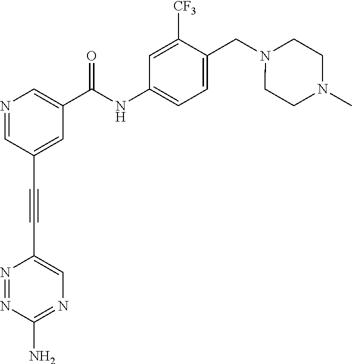 [SMILES: CN1CCN(Cc2ccc(NC(=O)c3cncc(C#Cc4cnc(N)nn4)c3)cc2C(F)(F)F)CC1]
or a pharmaceutically acceptable salt, hydrate, tautomer, or optical isomer thereof.

6. The compound of claim 1, wherein the compound is: 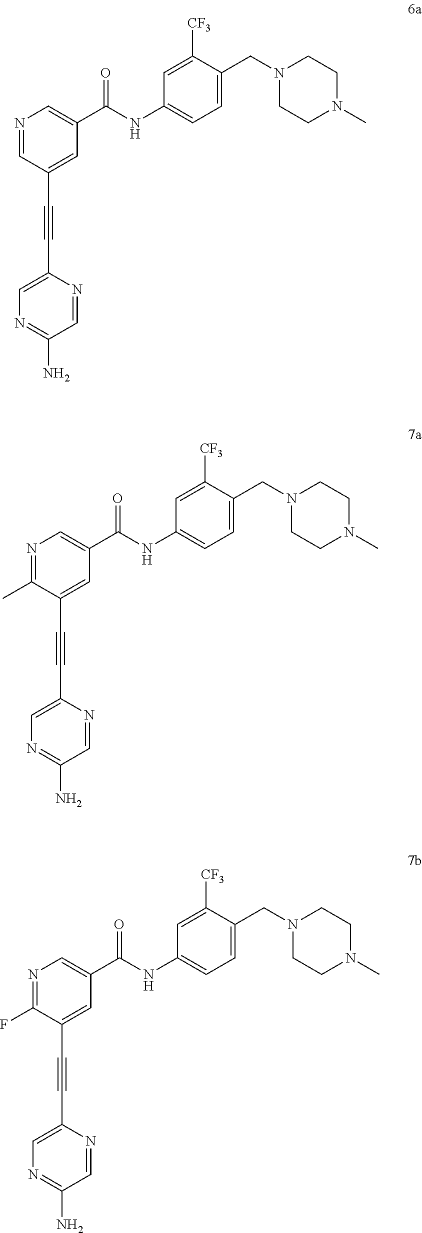 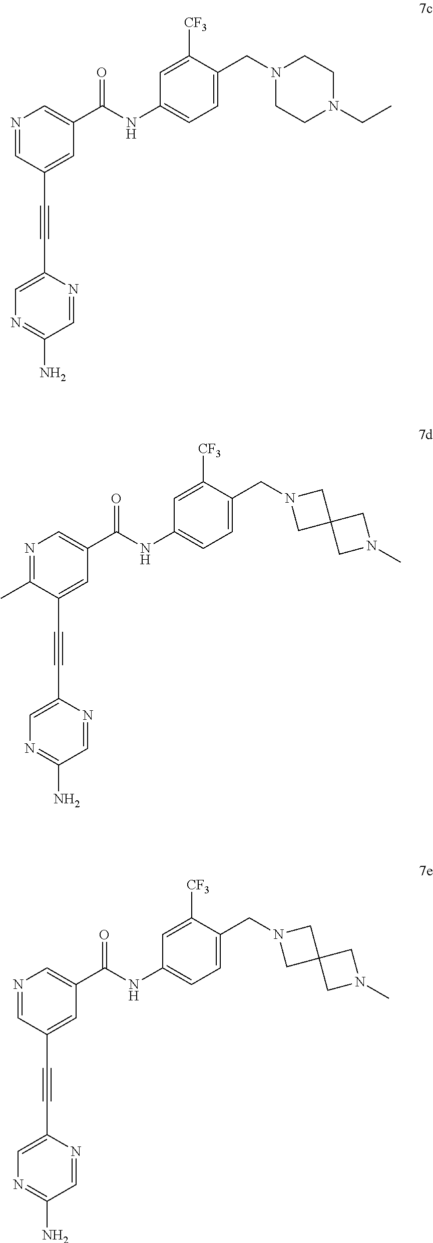 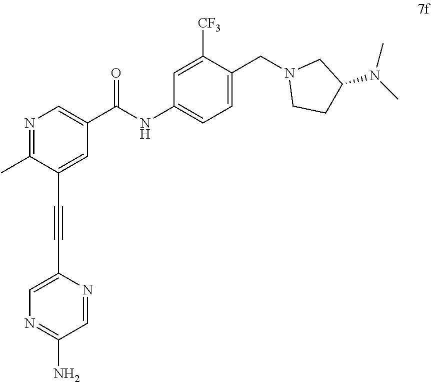 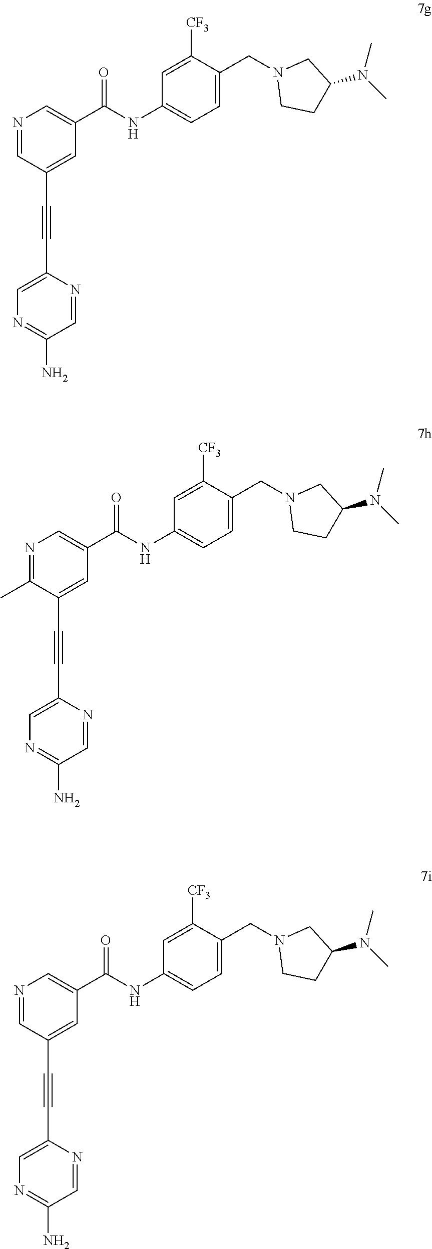 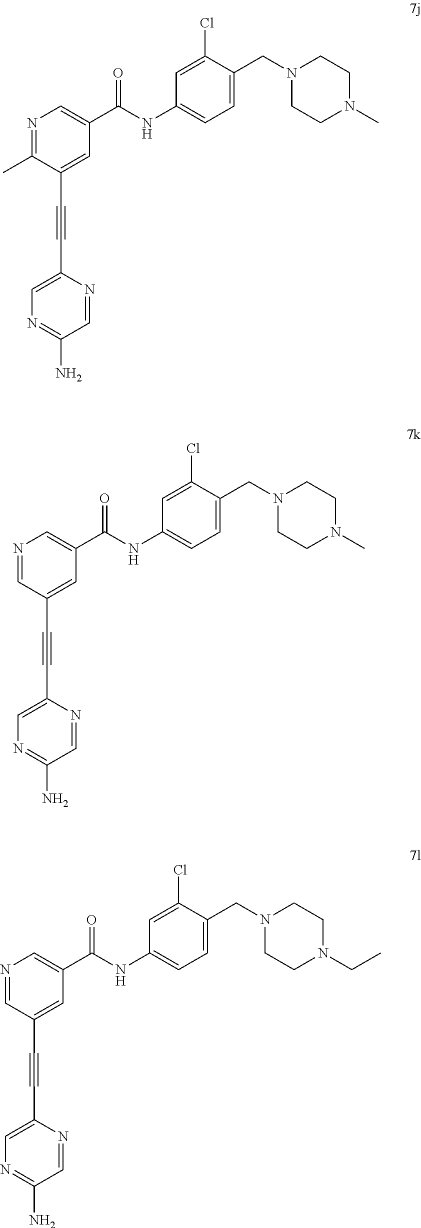 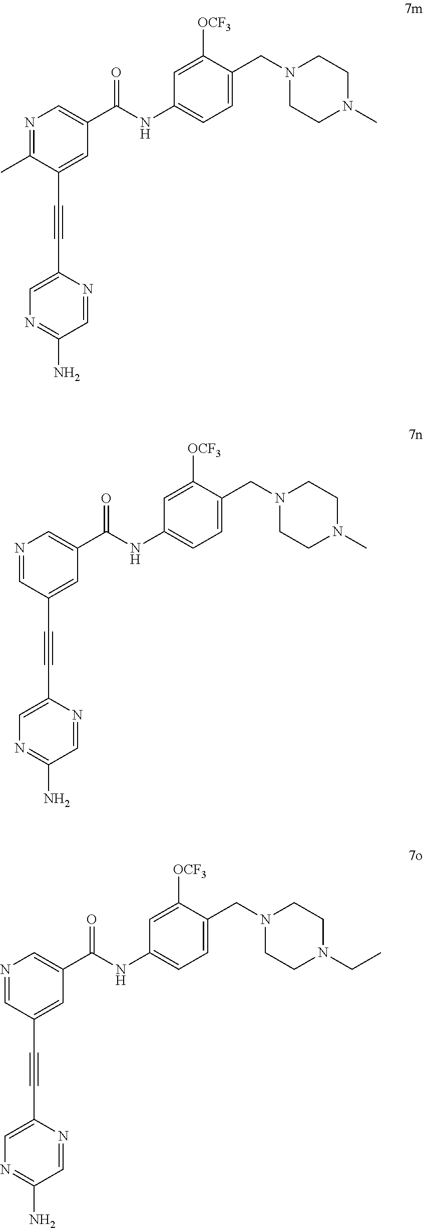 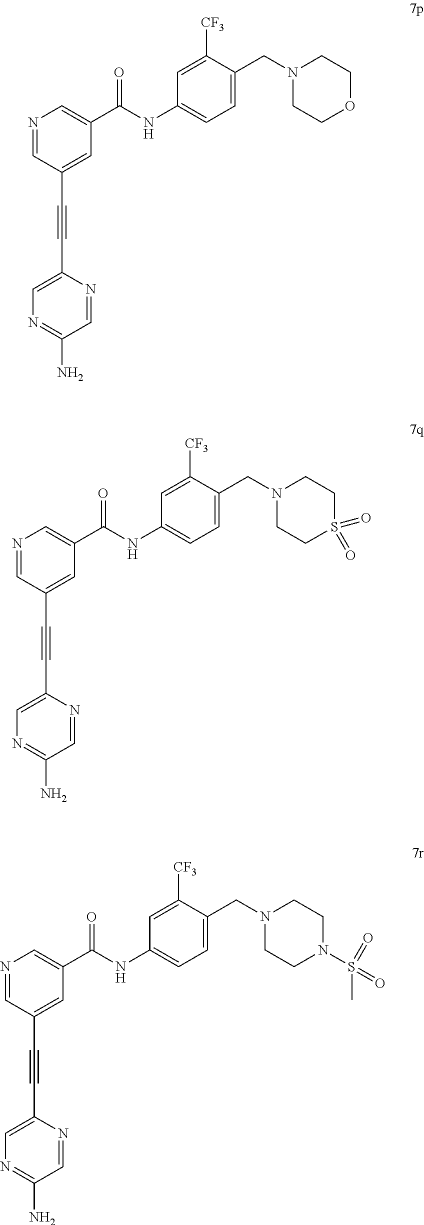 [SMILES: Nc1cnc(C#Cc2cncc(C(=O)Nc3ccc(CN4CCOCC4)c(C(F)(F)F)c3)c2)cn1 ; Nc1cnc(C#Cc2cncc(C(=O)Nc3ccc(CN4CCS(=O)(=O)CC4)c(C(F)(F)F)c3)c2)cn1 ; CS(=O)(=O)N1CCN(Cc2ccc(NC(=O)c3cncc(C#Cc4cnc(N)cn4)c3)cc2C(F)(F)F)CC1] 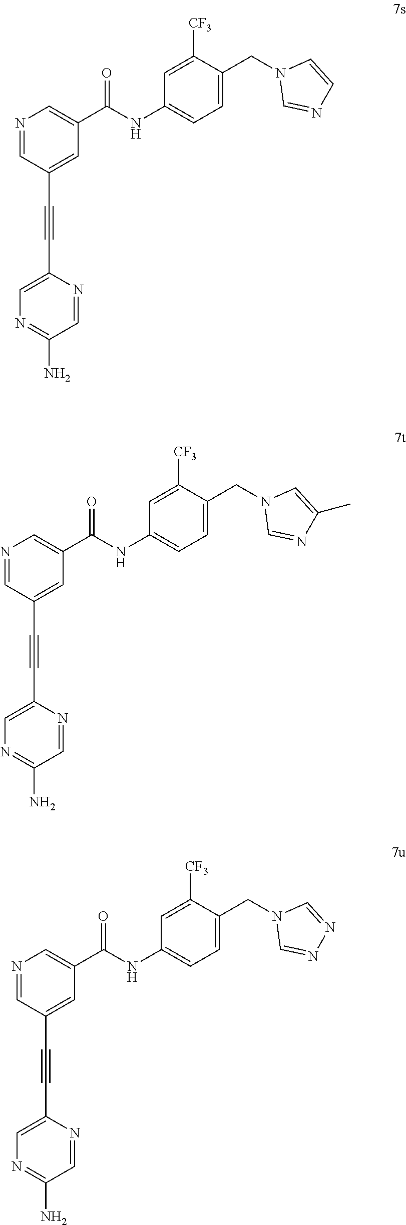 [SMILES: Nc1cnc(C#Cc2cncc(C(=O)Nc3ccc(Cn4ccnc4)c(C(F)(F)F)c3)c2)cn1 ; Cc1cn(Cc2ccc(NC(=O)c3cncc(C#Cc4cnc(N)cn4)c3)cc2C(F)(F)F)cn1 ; Nc1cnc(C#Cc2cncc(C(=O)Nc3ccc(Cn4cnnc4)c(C(F)(F)F)c3)c2)cn1] 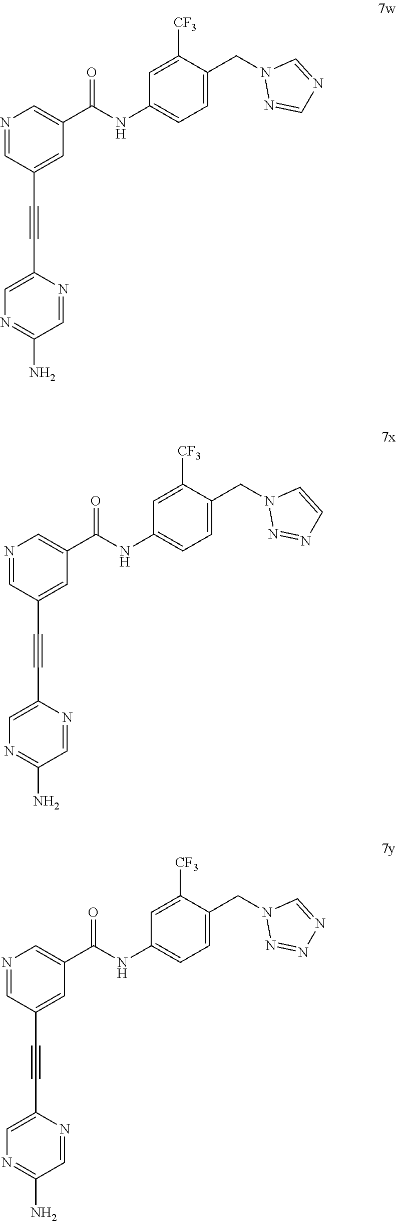 [SMILES: Nc1cnc(C#Cc2cncc(C(=O)Nc3ccc(Cn4cncn4)c(C(F)(F)F)c3)c2)cn1 ; Nc1cnc(C#Cc2cncc(C(=O)Nc3ccc(Cn4ccnn4)c(C(F)(F)F)c3)c2)cn1 ; Nc1cnc(C#Cc2cncc(C(=O)Nc3ccc(Cn4cnnn4)c(C(F)(F)F)c3)c2)cn1] 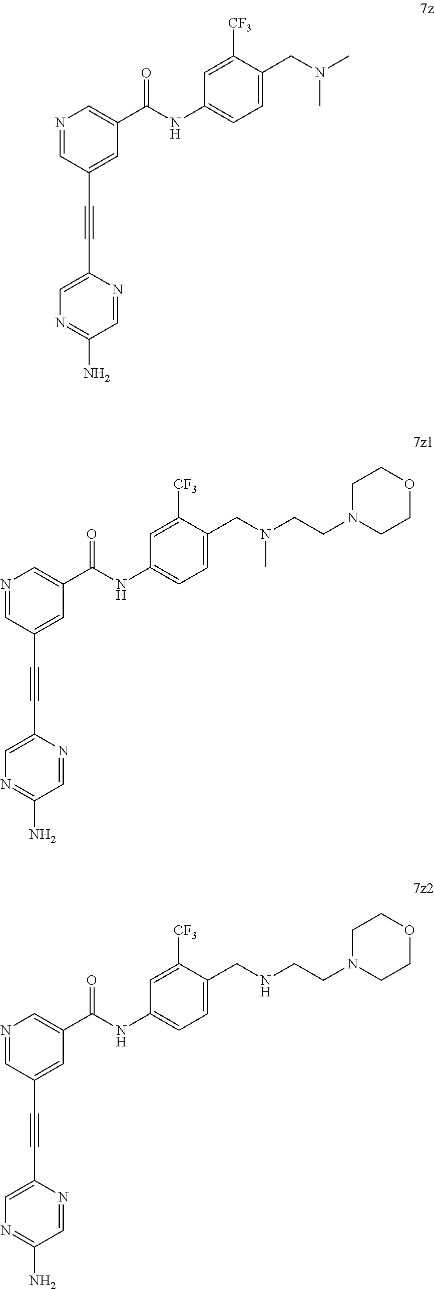 [SMILES: CN(C)Cc1ccc(NC(=O)c2cncc(C#Cc3cnc(N)cn3)c2)cc1C(F)(F)F ; CN(CCN1CCOCC1)Cc1ccc(NC(=O)c2cncc(C#Cc3cnc(N)cn3)c2)cc1C(F)(F)F ; Nc1cnc(C#Cc2cncc(C(=O)Nc3ccc(CNCCN4CCOCC4)c(C(F)(F)F)c3)c2)cn1] 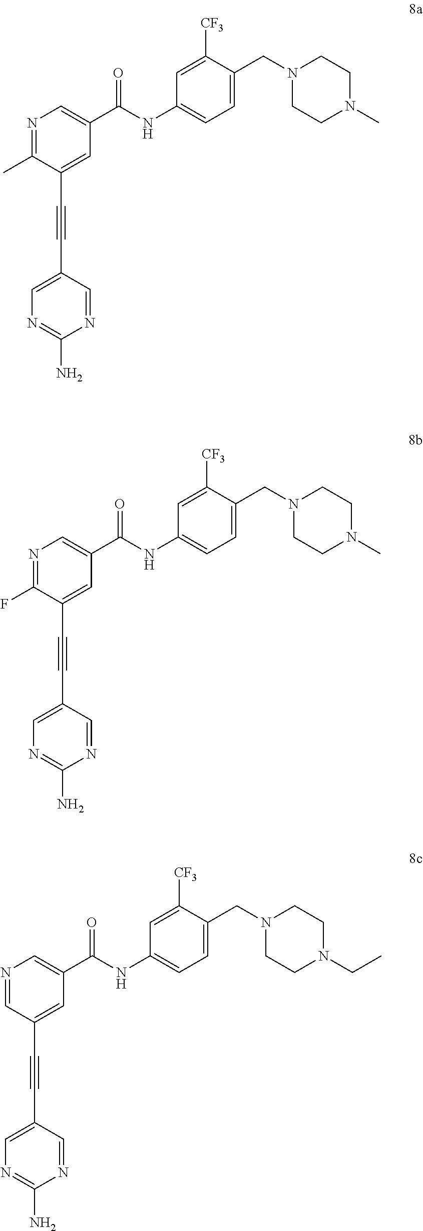 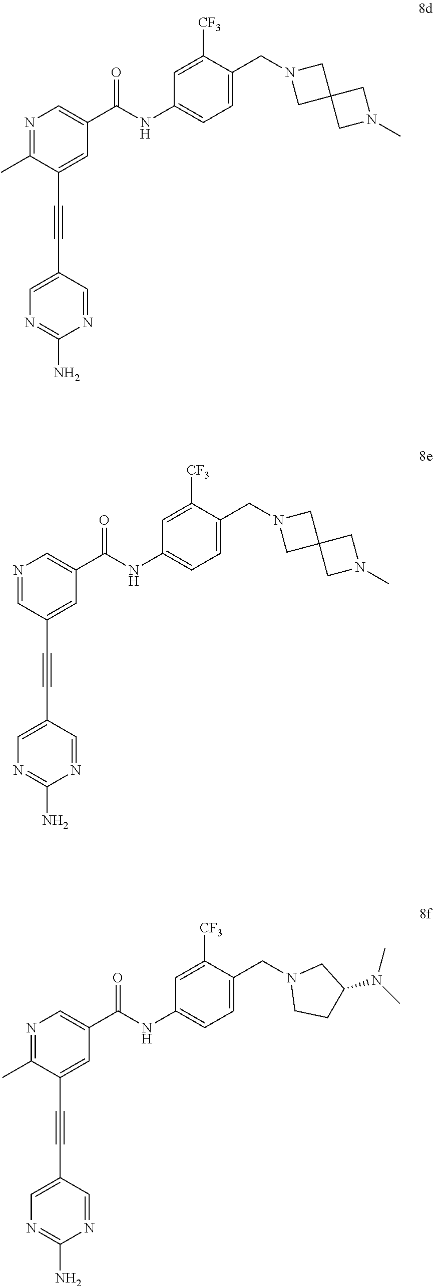 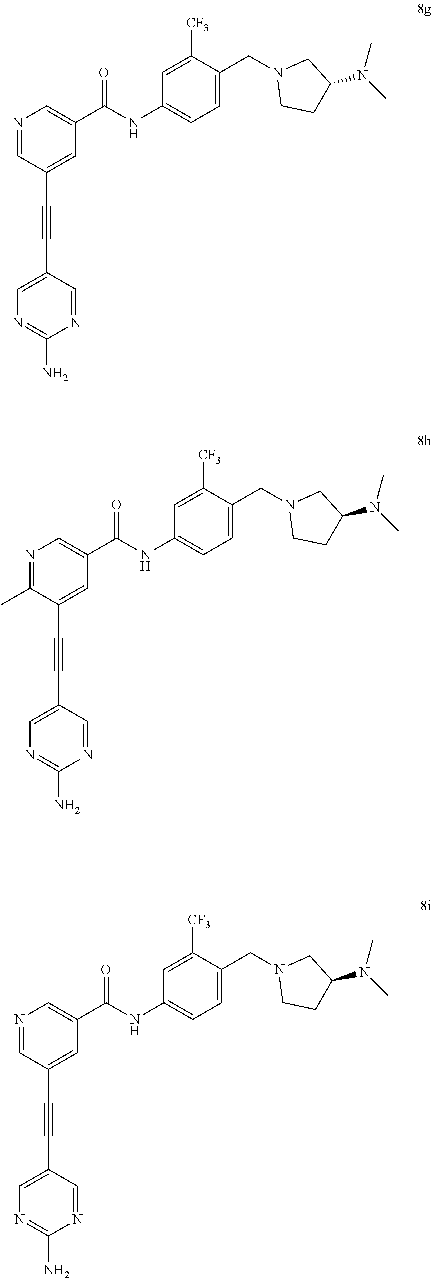 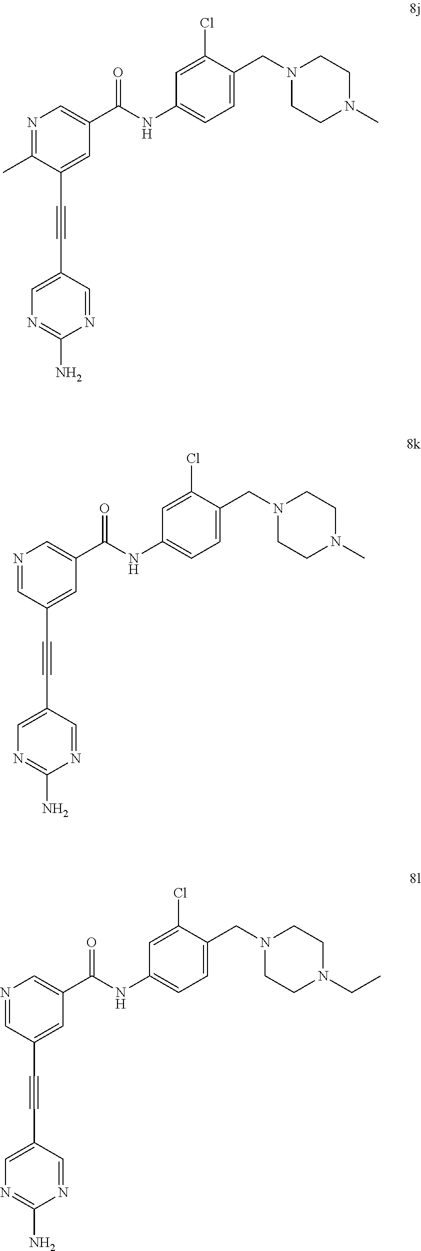 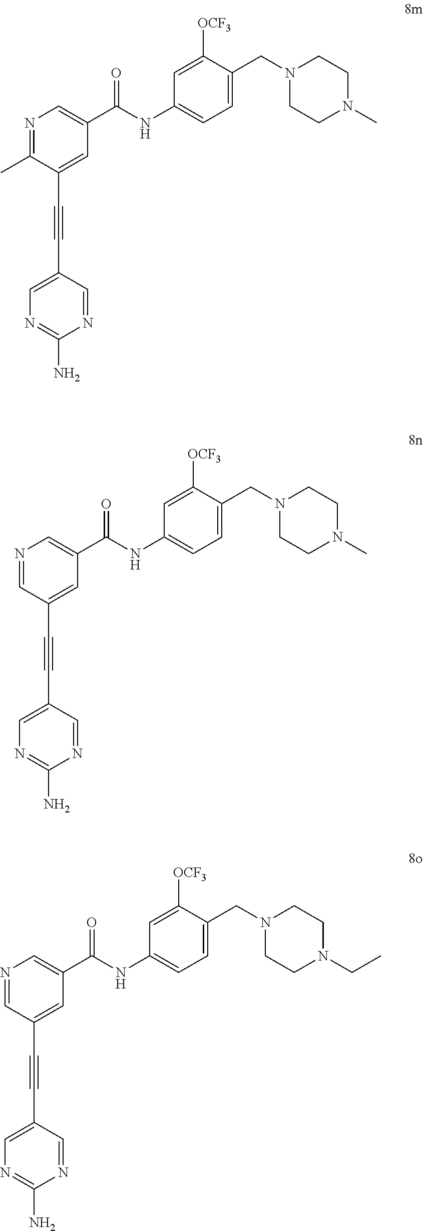 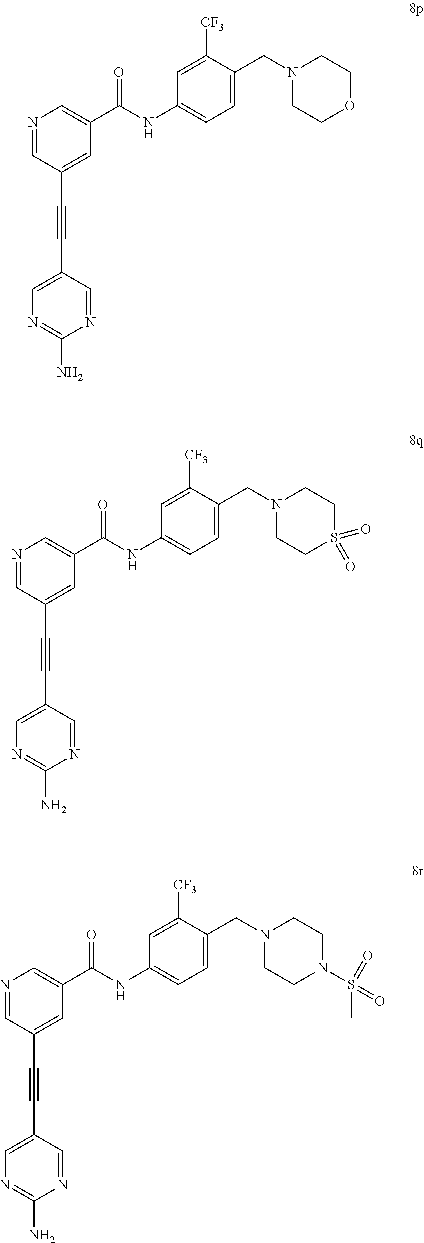 [SMILES: Nc1ncc(C#Cc2cncc(C(=O)Nc3ccc(CN4CCOCC4)c(C(F)(F)F)c3)c2)cn1 ; Nc1ncc(C#Cc2cncc(C(=O)Nc3ccc(CN4CCS(=O)(=O)CC4)c(C(F)(F)F)c3)c2)cn1 ; CS(=O)(=O)N1CCN(Cc2ccc(NC(=O)c3cncc(C#Cc4cnc(N)nc4)c3)cc2C(F)(F)F)CC1] 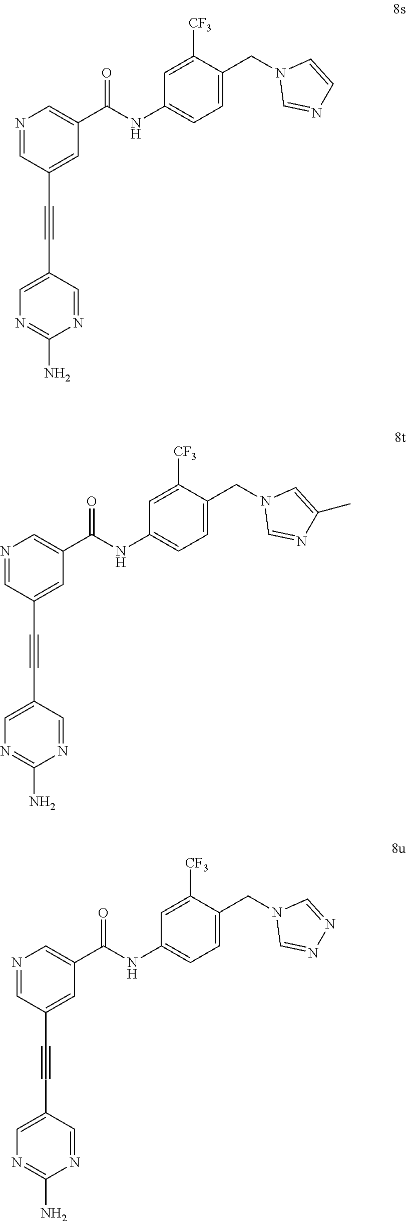 [SMILES: Nc1ncc(C#Cc2cncc(C(=O)Nc3ccc(Cn4ccnc4)c(C(F)(F)F)c3)c2)cn1 ; Cc1cn(Cc2ccc(NC(=O)c3cncc(C#Cc4cnc(N)nc4)c3)cc2C(F)(F)F)cn1 ; Nc1ncc(C#Cc2cncc(C(=O)Nc3ccc(Cn4cnnc4)c(C(F)(F)F)c3)c2)cn1] 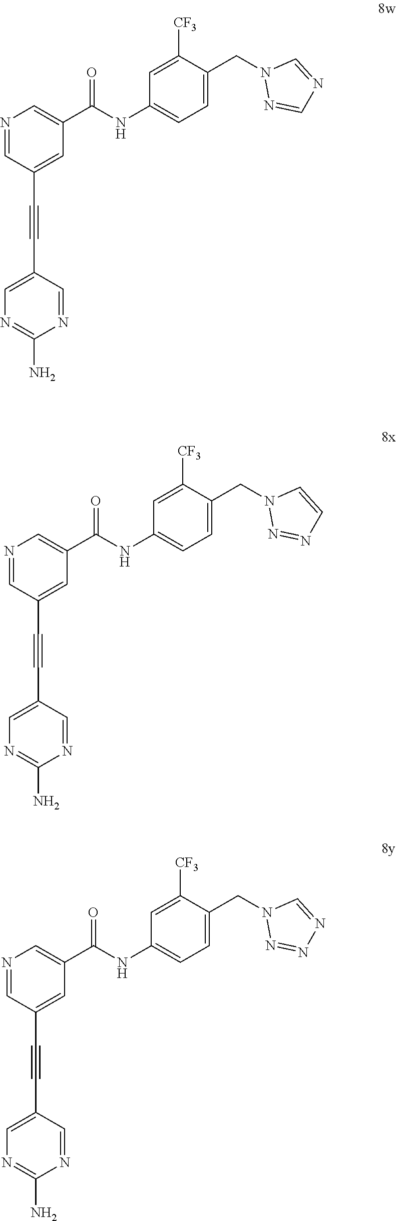 [SMILES: Nc1ncc(C#Cc2cncc(C(=O)Nc3ccc(Cn4cncn4)c(C(F)(F)F)c3)c2)cn1 ; Nc1ncc(C#Cc2cncc(C(=O)Nc3ccc(Cn4ccnn4)c(C(F)(F)F)c3)c2)cn1 ; Nc1ncc(C#Cc2cncc(C(=O)Nc3ccc(Cn4cnnn4)c(C(F)(F)F)c3)c2)cn1] 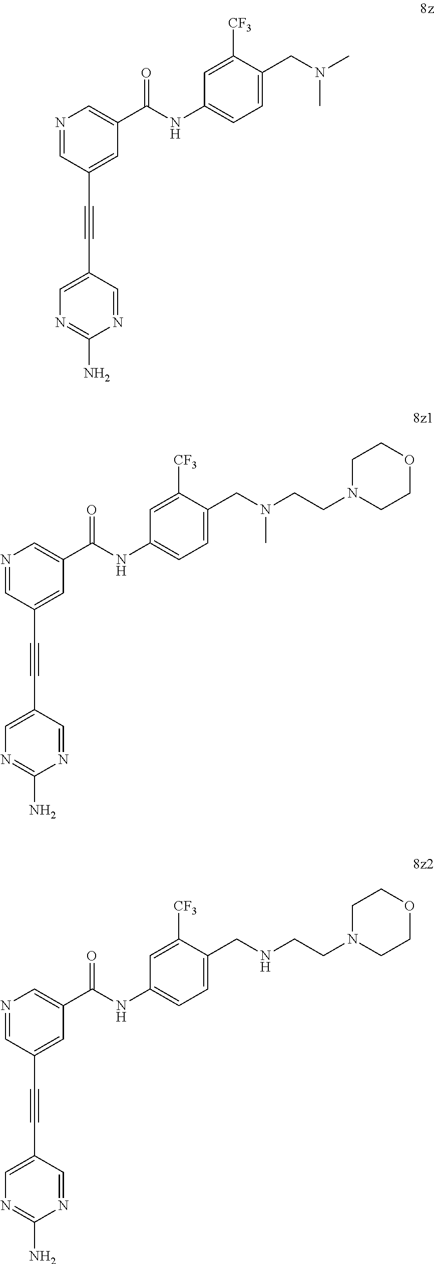 [SMILES: CN(C)Cc1ccc(NC(=O)c2cncc(C#Cc3cnc(N)nc3)c2)cc1C(F)(F)F ; CN(CCN1CCOCC1)Cc1ccc(NC(=O)c2cncc(C#Cc3cnc(N)nc3)c2)cc1C(F)(F)F ; Nc1ncc(C#Cc2cncc(C(=O)Nc3ccc(CNCCN4CCOCC4)c(C(F)(F)F)c3)c2)cn1] 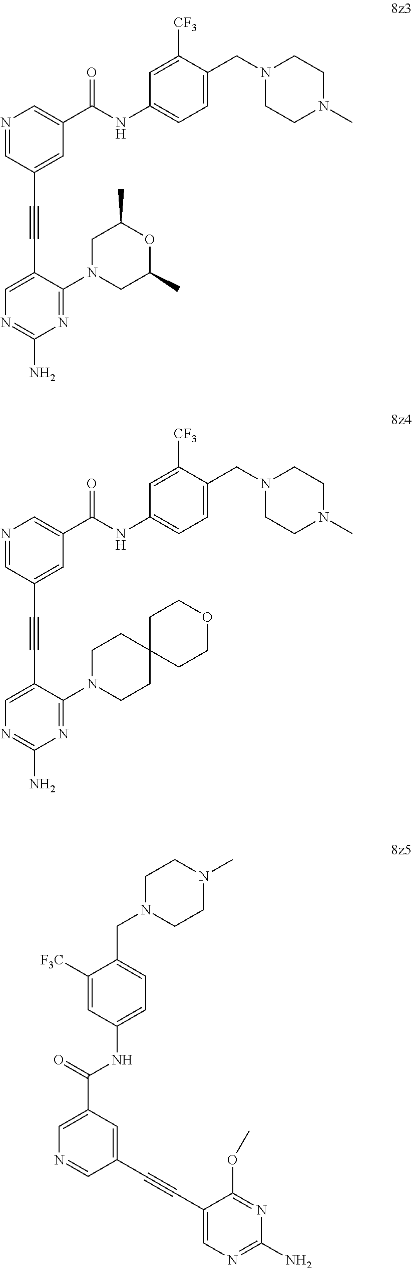 [SMILES: C[C@@H]1CN(c2nc(N)ncc2C#Cc2cncc(C(=O)Nc3ccc(CN4CCN(C)CC4)c(C(F)(F)F)c3)c2)C[C@H](C)O1 ; CN1CCN(Cc2ccc(NC(=O)c3cncc(C#Cc4cnc(N)nc4N4CCC5(CCOCC5)CC4)c3)cc2C(F)(F)F)CC1 ; COc1nc(N)ncc1C#Cc1cncc(C(=O)Nc2ccc(CN3CCN(C)CC3)c(C(F)(F)F)c2)c1] 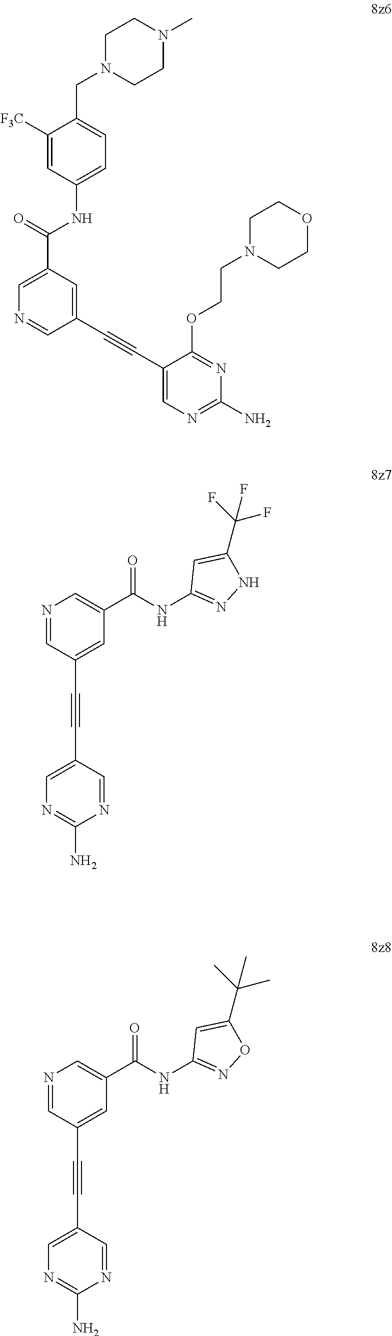 [SMILES: CN1CCN(Cc2ccc(NC(=O)c3cncc(C#Cc4cnc(N)nc4OCCN4CCOCC4)c3)cc2C(F)(F)F)CC1 ; Nc1ncc(C#Cc2cncc(C(=O)Nc3cc(C(F)(F)F)[nH]n3)c2)cn1 ; CC(C)(C)c1cc(NC(=O)c2cncc(C#Cc3cnc(N)nc3)c2)no1] 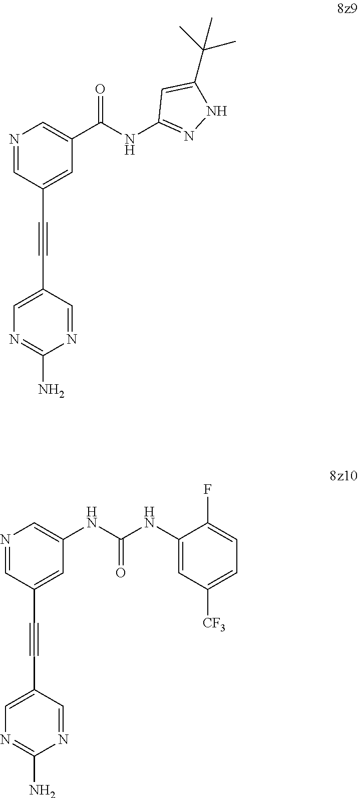 [SMILES: CC(C)(C)c1cc(NC(=O)c2cncc(C#Cc3cnc(N)nc3)c2)n[nH]1 ; Nc1ncc(C#Cc2cncc(NC(=O)Nc3cc(C(F)(F)F)ccc3F)c2)cn1] 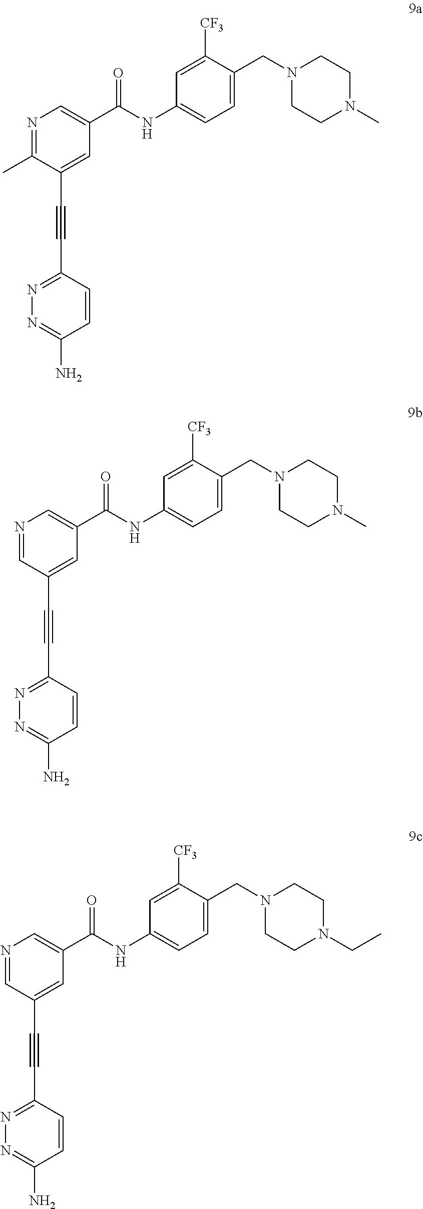 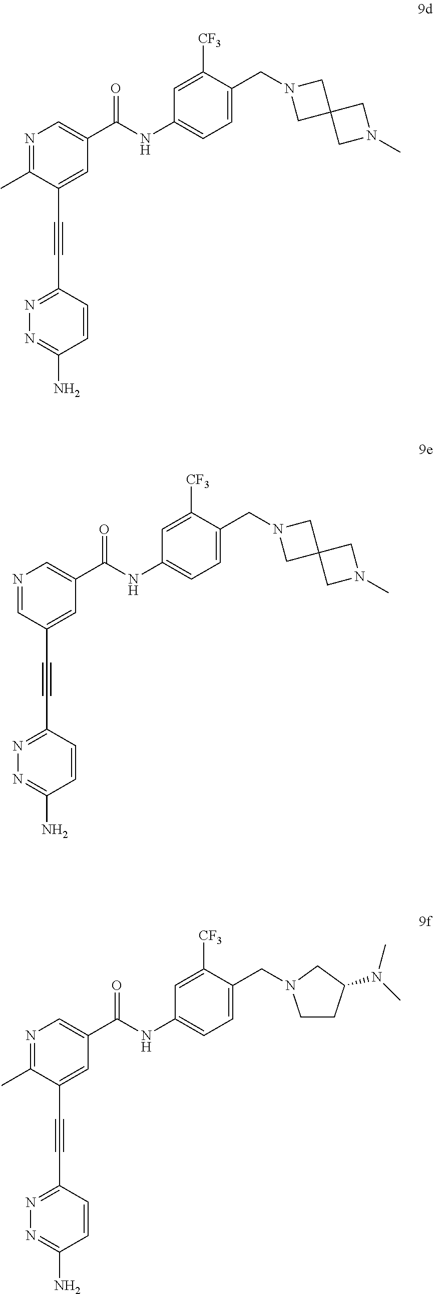 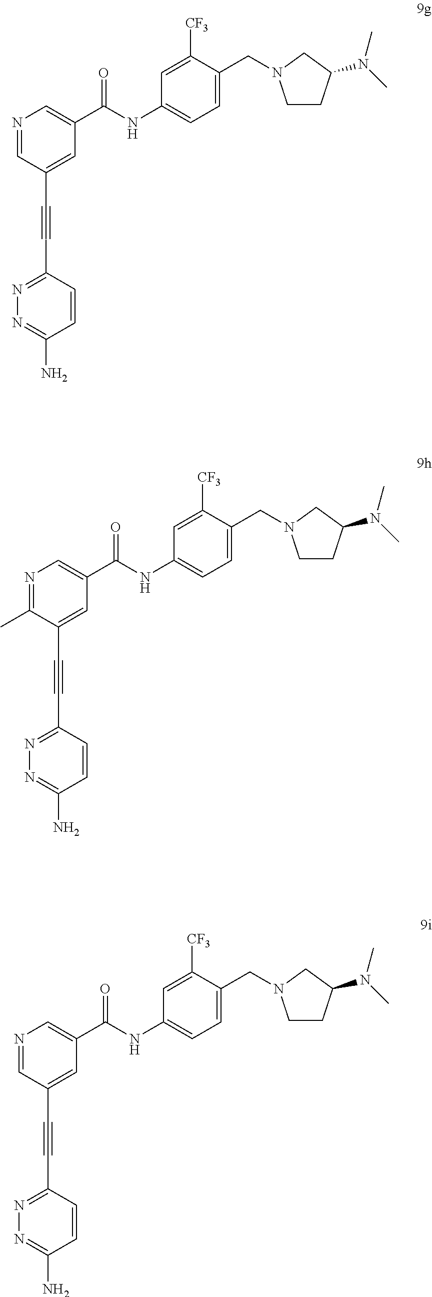 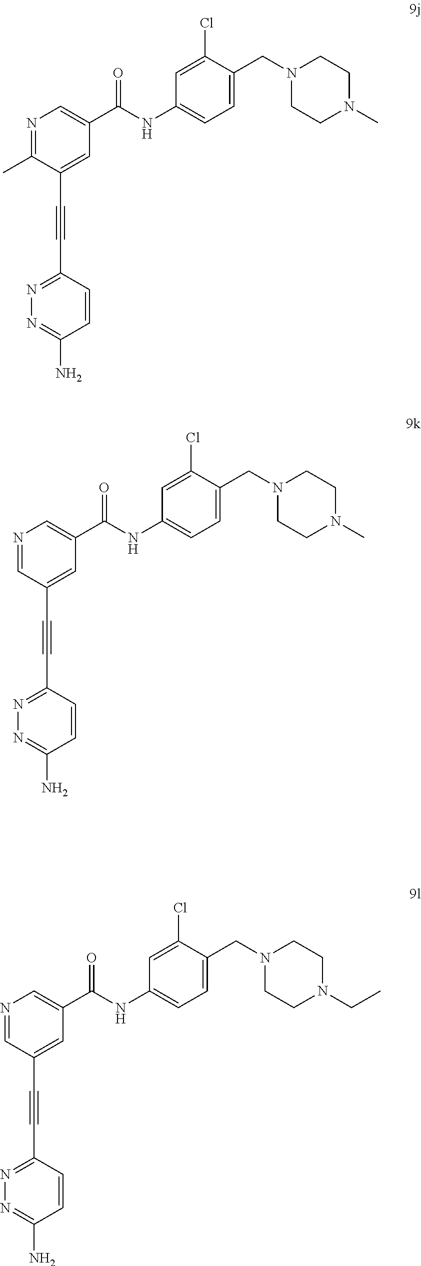 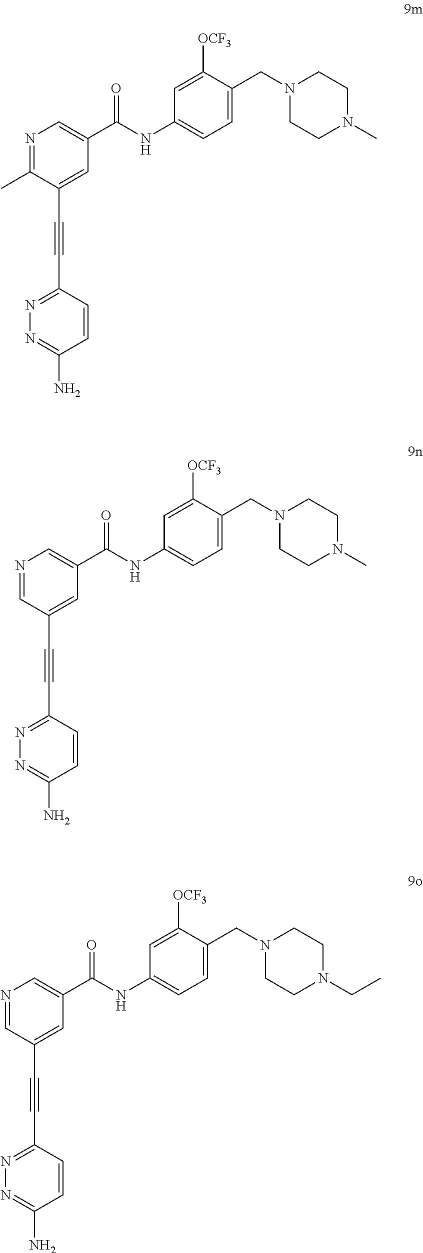 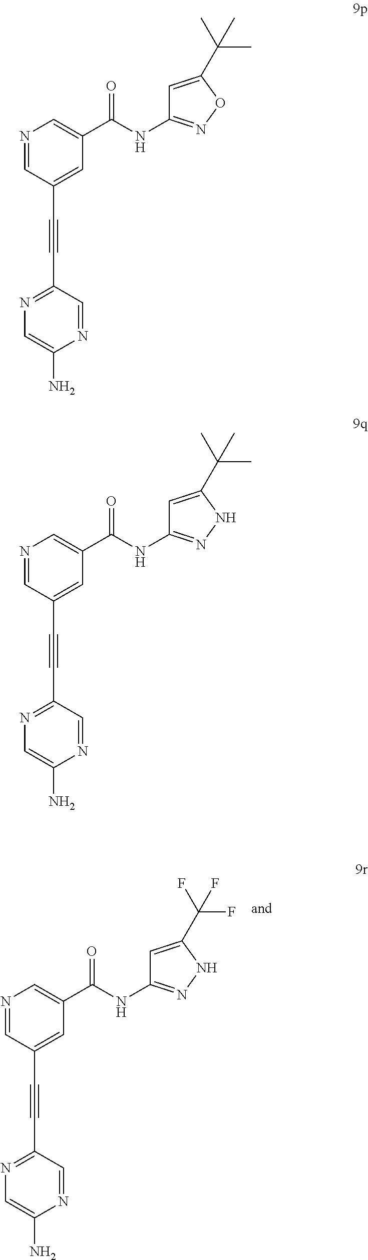 [SMILES: CC(C)(C)c1cc(NC(=O)c2cncc(C#Cc3cnc(N)cn3)c2)no1 ; CC(C)(C)c1cc(NC(=O)c2cncc(C#Cc3cnc(N)cn3)c2)n[nH]1 ; Nc1cnc(C#Cc2cncc(C(=O)Nc3cc(C(F)(F)F)[nH]n3)c2)cn1] 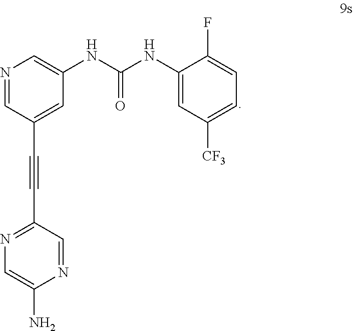 [SMILES: Nc1cnc(C#Cc2cncc(NC(=O)Nc3cc(C(F)(F)F)ccc3F)c2)cn1]
or a pharmaceutically acceptable salt, hydrate, tautomer, or optical isomer thereof.

7. A compound of formula (IA), or a pharmaceutically acceptable salt, hydrate, tautomer, or optical isomer thereof: 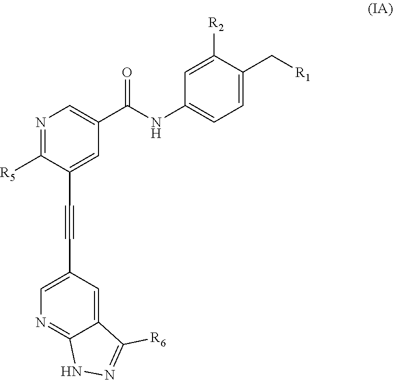
wherein
R₁ is NRₐR_{b}, wherein Rₐ and R_{b} each independently represents a group selected from H, alkyl, cycloalkyl, heterocycloalkyl, aryl, heteroaryl, —(CH₂)ₚ-alkyl, —(CH₂)ₚ-cycloalkyl, —(CH₂)ₚ-heterocycloalkyl, —(CH₂)ₚ—SO₂R_{c}, —(CH₂)ₚ—OR_{c}, and —(CH₂)ₚ—NRₐR_{b}, wherein Rₐ and R_{b} each independently is optionally substituted with at least one of alkyl, halo, OR_{c}, and NH₂; or
Rₐ and R_{b}, together with the nitrogen atom to which they are attached, form a 4- to 8-membered heterocycloalkyl group, which is a mono or a bicyclic ring, either of which is optionally substituted with a group selected from alkyl, cycloalkyl, halo, nitro, CN, SO₂R_{c}, OR_{d}, —(CH₂)ₚ—OR_{c} and NR_{c}R_{c};
R_{c} is H or alkyl;
R_{d} is H, halo, OH, CN, alkyl, —(CH₂)ₚ-alkyl, —(CH₂)ₚ-cycloalkyl, —(CH₂)ₚ-heterocycloalkyl, —(CH₂)ₚ—SO₂R_{d}, —(CH₂)ₚ—OR_{d}, —(CH₂)ₚ—(OR_{d})₂, or —(CH₂)ₚ—NRₐR_{b};
p is 0-5;
R₂ represents a group independently selected from alkyl, CF₃, OCF₃, and halo;
R₅ represents a group independently selected from H, alkyl, and halo;
R₆ represents a group independently selected from H, alkyl, ORₐ, and NRₐR_{b}, wherein Rₐ and R_{b} each independently represent a group selected from H, alkyl, cycloalkyl, heterocycloalkyl, aryl, heteroaryl, —(CH₂)ₚ-alkyl, —(CH₂)ₚ-cycloalkyl, —(CH₂)ₚ-heterocycloalkyl, —(CH₂)ₚ—SO₂R_{c}, —(CH₂)ₚ—OR_{c}, and —(CH₂)ₚ—NRₐR_{b}, wherein Rₐ and R_{b} each independently is optionally substituted with at least one of alkyl, halo, OR_{c}, and NH₂; or
Rₐ and R_{b}, together with the nitrogen atom to which they are attached, form a 4- to 8-membered heterocycloalkyl group, which is a mono or a bicyclic ring, either of which is optionally substituted with a group selected from alkyl, cycloalkyl, halo, nitro, CN, SO₂R_{c}, OR_{d}, —(CH₂)ₚ—OR_{c} and NR_{c}R_{c}, wherein R_{c}, R_{d}, and p are as defined above.

8. The compound of claim 7, wherein R₆ represents a group selected from

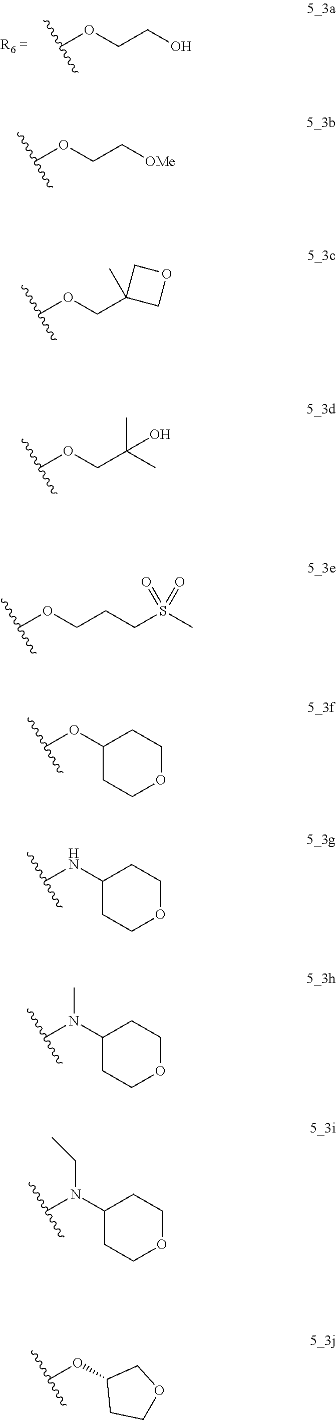

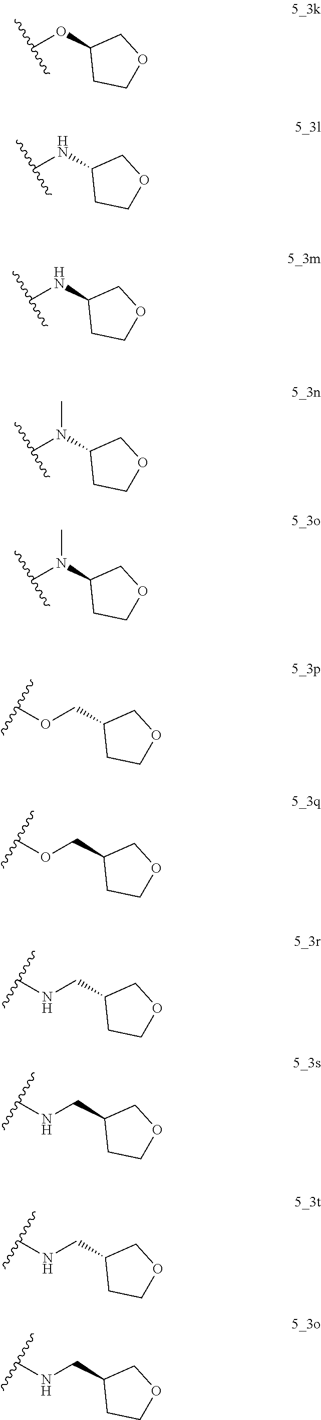

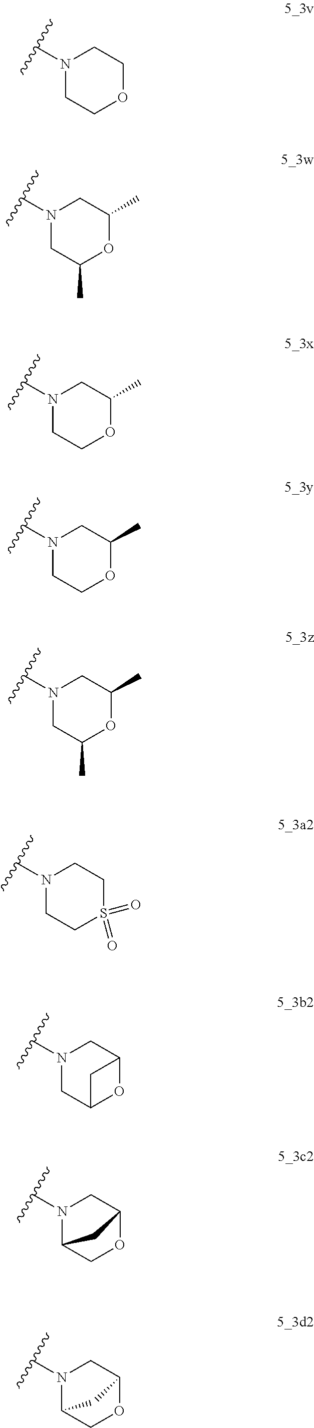

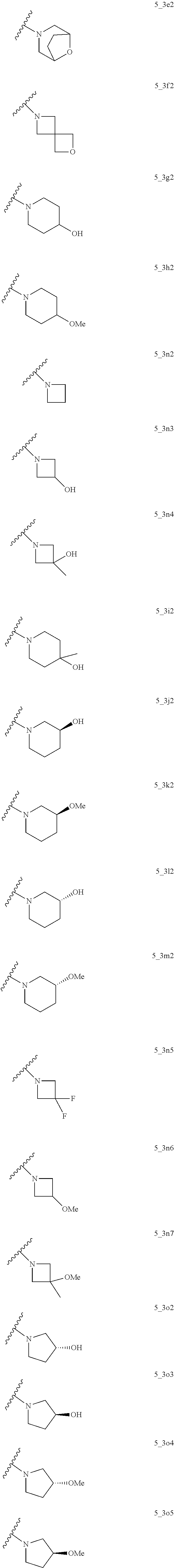

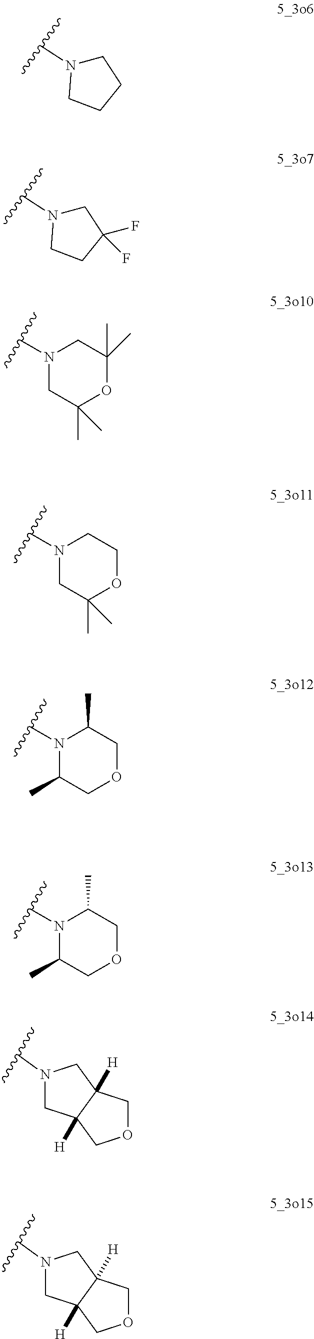

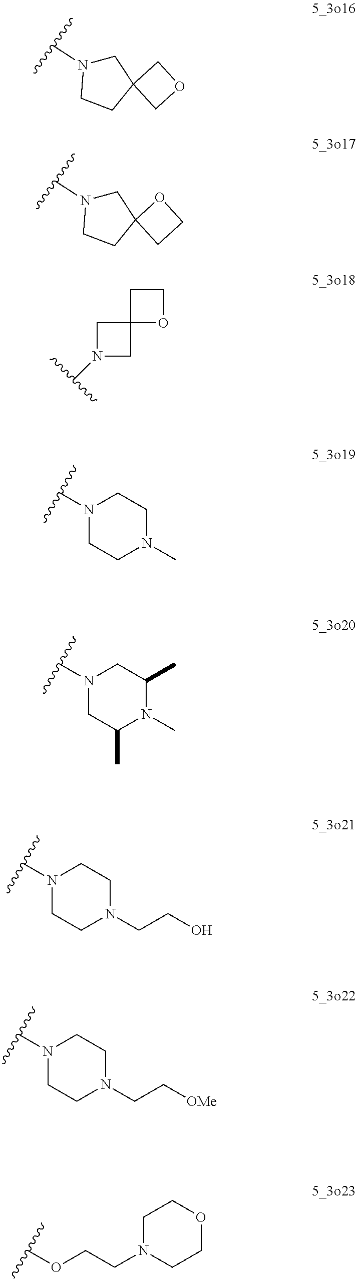

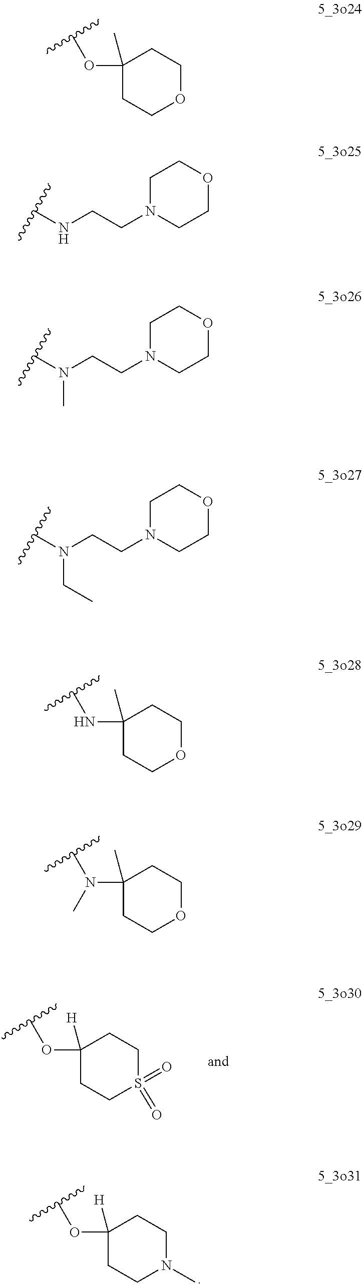

9. The compound of claim 7, wherein the compound is 5-((1H-pyrazolo[3,4-b]pyridin-5-yl)ethynyl)-N-(4-((4-methylpiperazin-1-yl)methyl)-3-(trifluoromethyl)phenyl)nicotinamide, 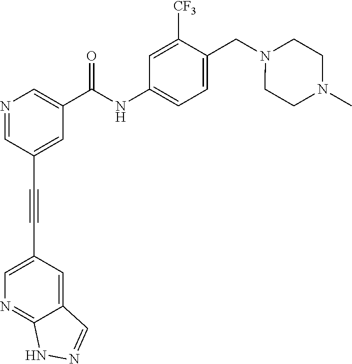 [SMILES: CN1CCN(Cc2ccc(NC(=O)c3cncc(C#Cc4cnc5[nH]ncc5c4)c3)cc2C(F)(F)F)CC1]
or a pharmaceutically acceptable salt, hydrate, tautomer, or optical isomer thereof.

10. The compound of claim 7, wherein the compound is 5-((3-methyl-1H-pyrazolo[3,4-b]pyridin-5-yl)ethynyl)-N-(4-((4-methylpiperazin-1-yl)methyl)-3-(trifluoromethyl)phenyl)nicotinamide, 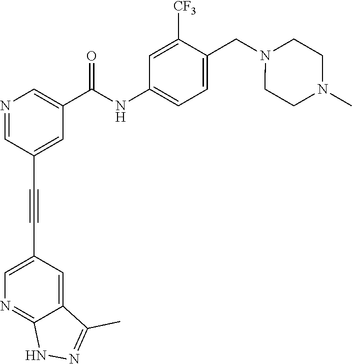 [SMILES: Cc1n[nH]c2ncc(C#Cc3cncc(C(=O)Nc4ccc(CN5CCN(C)CC5)c(C(F)(F)F)c4)c3)cc12]
or a pharmaceutically acceptable salt, hydrate, tautomer, or optical isomer thereof.

11. The compound of claim 7, wherein the compound is: 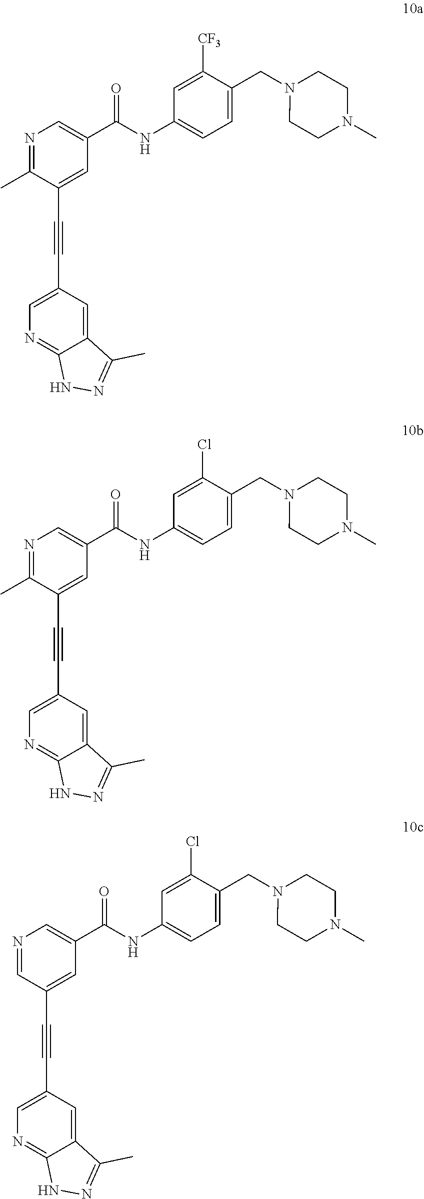 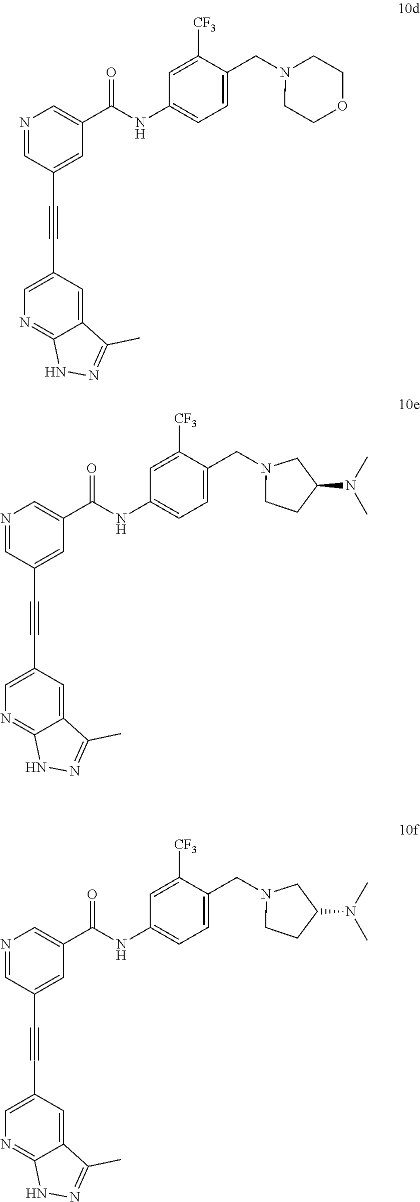 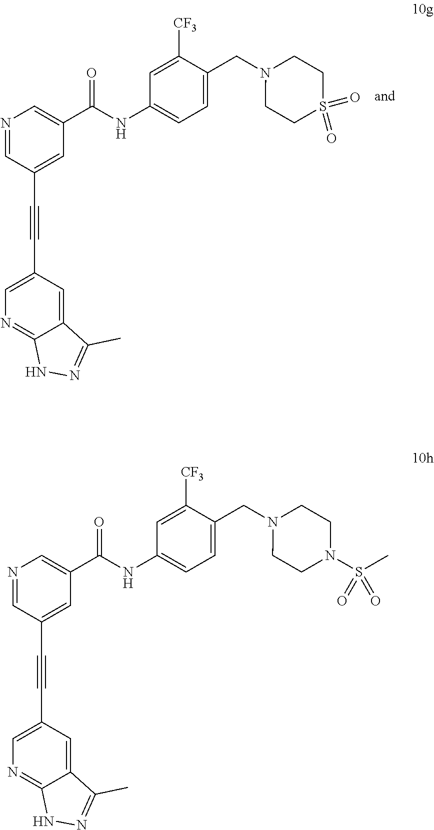 [SMILES: Cc1n[nH]c2ncc(C#Cc3cncc(C(=O)Nc4ccc(CN5CCS(=O)(=O)CC5)c(C(F)(F)F)c4)c3)cc12 ; Cc1n[nH]c2ncc(C#Cc3cncc(C(=O)Nc4ccc(CN5CCN(S(C)(=O)=O)CC5)c(C(F)(F)F)c4)c3)cc12]
or a pharmaceutically acceptable salt, hydrate, tautomer, or optical isomer thereof.

12. The compound of any one of claims 1-11, wherein the compound selectively inhibits a rearranged during transfection (RET) kinase mutant.

13. The compound of claim 12, wherein the RET kinase mutant is G810C, G810A, G810R, G810S, or V804M.

14. A pharmaceutical composition comprising a compound of formula (I) or a pharmaceutically acceptable salt, hydrate, tautomer, or optical isomer thereof, and a pharmaceutically acceptable carrier, excipient, or diluent.

15. A pharmaceutical composition comprising a compound of formula (IA) or a pharmaceutically acceptable salt, hydrate, tautomer, or optical isomer thereof, and a pharmaceutically acceptable carrier, excipient, or diluent.

16. A method of treating or inhibiting a rearranged during transfection (RET) kinase-altered cancer, wherein the method comprises inhibiting RET kinase mutants by administering to a patient in need thereof a therapeutically effective amount of a compound of formula (I) of any one of claims 1-6, optionally as a pharmaceutical composition comprising the compound and a pharmaceutically acceptable carrier, excipient, or diluent, whereupon the RET kinase-altered cancer in the patient is treated or inhibited.

17. The method of claim 16, wherein the RET kinase mutant is G810.

18. The method of claim 16, wherein the RET kinase-altered cancer is lung cancer, thyroid cancer, pancreatic cancer, or breast cancer.

19. A method of treating or inhibiting a rearranged during transfectioni (RET) kinase-altered cancer, wherein the method comprises inhibiting RET kinase mutants by administering to a patient in need thereof a therapeutically effective amount of a compound of formula (IA) of any one of claims 7-11, optionally as a pharmaceutical composition comprising the compound and a pharmaceutically acceptable carrier, excipient, or diluent, whereupon the RET kinase-altered cancer in the patient is treated or inhibited.

20. The method of claim 19, wherein the RET kinase mutant is G810.

21. The method of claim 19, wherein the RET kinase-altered cancer is lung cancer, thyroid cancer, pancreatic cancer, or breast cancer.

22. A method of inhibiting a rearranged during transfection (RET) kinase mutants in a patient in need thereof, which method comprises administering to the patient a therapeutically effective amount of a compound of any one of claims 1-11, optionally as a pharmaceutical composition comprising the same and a pharmaceutically acceptable carrier, excipient, or diluent, whereupon RET kinase mutants in the patient are inhibited.

23. The method of claim 22, wherein the patient has RET kinase-altered cancer.

24. The method of claim 23, wherein the RET kinase-altered cancer is lung cancer, thyroid cancer, pancreatic cancer, or breast cancer.
